(19) United States
(12) Patent Application Publication
(10) Pub. No.: US 2022/0160703 A1
(43) Pub. Date: May 26, 2022

(54) TUMOR-SELECTIVE COMBINATION THERAPY

(71) Applicants: The Board of Regents of The University of Texas System, Austin, TX (US); Indiana University Research and Technology Corporation, Indianapolis, IN (US); The Board of Trustees of the University of Illinois, Urbana, IL (US)

(72) Inventors: Yang-Xin FU, Dallas, TX (US); Xiumei HUANG, Indianapolis, IN (US); David BOOTHMAN, Deceased (US); Paul J. HERGENROTHER, Urbana, IL (US); Xiaoguang LI, Dallas, TX (US); Lingxiang JIANG, Indianapolis, IN (US)

(73) Assignees: The Board of Regents of The University of Texas System, Austin, TX (US); Indiana University Research and Technology Corporation, Indianapolis, IN (US); The Board of Trustees of the University of Illinois, Urbana, IL (US)

(21) Appl. No.: 17/440,787

(22) PCT Filed: Mar. 18, 2020
(86) PCT No.: PCT/US2020/023250

§ 371 Date: Sep. 19, 2021

Related U.S. Application Data
(60) Provisional application No. 62/819,870, filed on Mar. 18, 2019.

(51) Int. Cl. A61K 31/4745; A61K 31/352; A61K 39/395; A61K 45/06; A61P 35/00

(52) U.S. Cl. CPC A61K 31/4745; A61K 31/352; A61P 35/00; A61K 45/06; A61K 39/3955

(57) ABSTRACT

The therapies described herein can be selectively lethal toward a variety of different cancer cell types and cancer conditions in a subject. The combination therapies described herein can be useful for the management, treatment, control, or adjunct treatment of diseases, where the selective lethality is beneficial in immunotherapy, particularly where the disease is accompanied by elevated levels of NQO1. In particular, embodiments where an immunotherapy, such as a heckpoint inhibitor, are combined with a NQO1 bioactivatable drug.

### PRIORITY CLAIM

This application claims benefit of priority to U.S. Provisional Application Ser. No. 62/819,870, filed Mar. 18, 2019, the entire contents of which are hereby incorporated by reference.

### GOVERNMENT SUPPORT

This invention was made with government support under contract number R01 CA102792-18 awarded by the National Institutes of Health. The government has certain rights in the invention.

### BACKGROUND

### I. Field

The field of the disclosure relates generally to medicinal chemistry, medicine, oncology, chemotherapy and immunotherapy. More specifically, it pertains to the use of NQO1-bioactivable drugs in combination with checkpoint inhibitors in the treatment of cancers.

### II. Related Art

Anti-Programmed Cell Death Protein 1 (PD-1) and its ligand Programmed Death-Ligand 1 (PDL1) monoclonal antibodies have demonstrated unprecedented durable responses across several different cancer types, and these initial clinical successes have highlighted the field of cancer immunotherapy. Blocking the interaction between PD-1 and its ligand PD-L1 result in increased T cell survival and proliferation and reduced T cell exhaustion, restoring cytotoxic T cell function to promote an antitumor immune response (Topalian et al., 2015; Pauken and Wherry, 2015). Unfortunately, only a minority of patients treated with anti-PD1/PD-L1 agents have durable responses (Sharma et al., 2017). While other checkpoints may contribute to low response and relapse, additional checkpoint blockades on T cells have not improved response rates (Jenkins et al., 2018; Garber, 2018). The dysfunctional antigen presenting cells inside tumor microenvironment might limit optimal activation of T cells (Lee et al., 2009). While tumor antigens might be available inside tumor environment, lack of proper activation of antigen processing and presentation might limit tumor-specific T cells be reactivated.

The generation of effective adaptive immunity requires the coordinative innate immune response, including sensing of danger or damage signals to activate innate cells (i.e., dendritic cells macrophages and natural killer cells), antigen processing and presentation, type I IFN production and cross-priming of T cells (Woo and Corrales, 2015). In general, these danger or damage signals are recognized by extracellular and intracellular pattern recognition receptors (PRRs) expressed by innate immune cells, and promote the uptake of antigens, activate antigen-presenting cells (APCs), and facilitate the interaction between APCs and damaged cells (Takeuchi and Akira, 2010). However, these critical properties of normal innate immune responses are often corrupted in the tumor microenvironment (Patel and Minn, 2018). For example, cancers pervasively favor the survival of tumor clones lacking or unable to present adequate neoantigens; tumors also prefer PRRs signals or dysfunctional innate immune cells that promote cancer inflammation rather than priming an adaptive response (Lee et al., 2009; Hernandez et al., 2016; Grivennikov et al., 2010).

Since tumors exhibit impaired innate sensing that favors an immunosuppressive microenvironment, an important consideration in improving checkpoint blockade is to enhance innate signals in the tumor microenvironment. One possible approach to achieve this goal involves the induction of immunogenic cell death (ICD) within the tumor microenvironment (Patel and Minn, 2018). For example, a number of DNA-damaging or DNA repair inhibiting chemotherapies (such as radiotherapy, anthracyclines and oxaliplatin) elicit ICD (Garg et al., 2017). ICD is characterized by release of a series of immunostimulatory damage-associated molecular patterns (DAMPs) such as high mobility group box 1 (HMGB1) protein, extracellular ATP, cytoplasmic calreticulin, and endogenous nucleic acids by the dying tumor cells (Sistigu et al., 2014). These DAMPs are recognized by their cognate PRRs expressed by innate cells. This DAMP/PRR signaling alters the inflammatory microenvironment and/or stimulates neoantigen production, and attracts and activates APCs to activate T cells, which are now licensed to attack the tumor (Galluzzi et al., 2015). Thus, the immunostimulatory properties make ICD-inducing agents attractive candidates for combination immunotherapy. However, only a few cytotoxic drugs have been identified as ICDs inducers, and the general toxicities, immune suppressive nature, and lack of tumor selectivity limit their use. Thus, it is highly desirable to explore whether “targeted” agents can more specifically increase innate sensing and subsequently expand the benefits of anti-PD1/PD-L1 treatment.

NAD(P)H:quinone oxidoreductase 1 (NQO1) is a cytosolic two-electron oxidoreductase which is upregulated in many human cancers (Li et al., 1995), including colorectal cancer, lung cancer, melanoma, cholangiocarcinoma, and pancreatic cancer (Oh et al., 2016). High-level expression of NQO1 is associated with late clinical stage, poor prognosis and lymph node metastasis (Li et al., 2015; Ma et al., 2014). NQO1 bioactivatable drugs, including β-lapachone (β-lap, in clinical form, ARQ761), have a unique quinone structure which can be catalyzed by NQO1 to generate reactive oxygen species (ROS) (Huang et al., 2016). In general, one mole of β-lap generates ˜120 moles of superoxide, consuming ˜60 moles of NAD(P)H in ˜2 min (Pink et al., 2000). NQO1 is overexpressed in tumor cells and catalase, a hydrogen peroxide (H₂O₂) scavenging enzyme, is lost in tumor tissues versus normal tissue (Doskey et al., 2016). High NQO1:Catalase ratios in human cancers can offer an optimal therapeutic window for the use of NQO1 ‘bioactivatable’ drugs, while low expression ratios protect normal tissue. The intensive tumor specific ROS production leads to extensive oxidative DNA lesions and tumor selective cell death (Huang et al., 2012). It has been demonstrated that NQO1 bioactivable β-lap causes unrepaired DNA damage and cell death and synergizes with PARP1 inhibitors and radiotherapy in xenograft models (Huang et al., 2016; Li et al., 2016). β-Lap is currently being tested in monotherapy or in combination with the other chemodrugs in patients with NQO1+ solid tumors (ClinicalTrials.gov identifiers NCT02514031 and NCT01502800). However, evaluations of the antitumor efficacy β-lap were mainly carried out in vitro and in immunodeficient mouse models, and improving therapy often focused on enhanced direct tumor killing with little attention to adaptive immunity. The inventors recently demonstrated that β-lap trigger immunogenic cell death (ICD) and induce damage-associated molecular patterns (DAMPs) release that activates the host TLR4/MyD88/type I interferon pathway and Batf3 dendritic cell-dependent cross-priming to bridge innate and adaptive immune responses against the NQO1 positive tumors (Li et al., 2019). Furthermore, they found that β-lap triggers innate sensing within the tumor microenvironment to overcome checkpoint blockade resistance in well-established tumors (Li et al., 2019). Isobutyl-deoxynyboquinone (IB-DNQ) is a novel selective substrate for NAD(P)H:quinone oxidoreductase (NQO1), an enzyme overexpressed in many solid tumors. IB-DNQ is a promising and potent anti-cancer agent that targets NQO1 positive solid cancers (Lundberg et al., 2017).

### SUMMARY

Thus, in accordance with the present disclosure, there is provided a method of killing or inhibiting the growth of cancer cells in a patient having cancer comprising administering a NQO1 bioactivatable drug in combination with a checkpoint inhibitor. Also provided is a use of a NQO1 bioactivatable drug in combination with a second agent, wherein the second agent is a checkpoint inhibitor, for the manufacture of a medicament for killing or inhibiting the growth of cancer cells in a patient that has cancerous cells, wherein the medicament comprises an effective lethal or inhibitory amount of the NQO1 bioactivatable drug and the checkpoint inhibitor. Also provided is a use of a NQO1 bioactivatable drug in combination with a second agent, wherein the second agent is a checkpoint inhibitor, in the treatment of a patient that has cancer. The methods may further comprise an additional anti-cancer therapy, such as a chemotherapy, a radiotherapy, an immunotherapy, a toxin therapy or surgery.

The cancer cells may have base excision repair (BER) defects or vulnerabilities due to faulty DNA repair processes. The BER defect or vulnerability may comprise defective levels of X-ray cross complementing 1 or XRCC1 gene/protein/enzyme. The NQO1 bioactivatable drug may be used in combination with a small molecule checkpoint inhibitor or an antibody checkpoint inhibitor. The NQO1 bioactivatable drug may be used in combination with an inhibitor of PD-1 or CTLA-4. The NQO1 bioactivatable drug may be β-lapachone or a DNQ compound. The methods may further comprise an additional chemotherapeutic agent or radiotherapy. The cancer cells may have elevated levels of NQO1. The cancer cells may be in the form of a solid tumor. The cancer cells may be non-small cell lung cancer cells, prostate cancer cells, pancreatic cancer cells, breast cancer cells, head and neck cancer cells, or colon cancer cells.

The NQO1 bioactivatable drug may be:

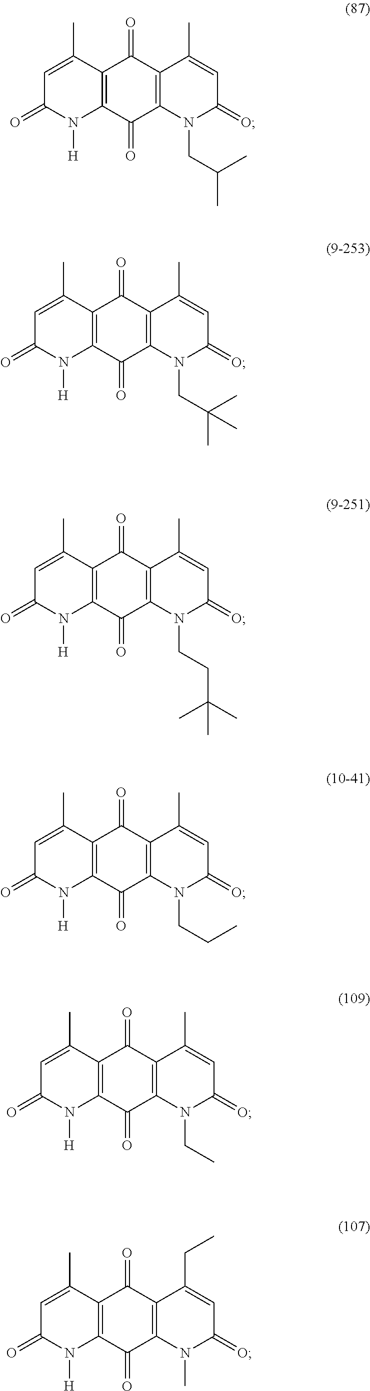 [SMILES: Cc1cc(=O)[nH]c2c1C(=O)c1c(C)cc(=O)n(CC(C)C)c1C2=O ; Cc1cc(=O)[nH]c2c1C(=O)c1c(C)cc(=O)n(CC(C)(C)C)c1C2=O ; Cc1cc(=O)[nH]c2c1C(=O)c1c(C)cc(=O)n(CCC(C)(C)C)c1C2=O ; CCCn1c2c(c(C)cc1=O)C(=O)c1c(C)cc(=O)[nH]c1C2=O ; CCn1c2c(c(C)cc1=O)C(=O)c1c(C)cc(=O)[nH]c1C2=O ; CCc1cc(=O)n(C)c2c1C(=O)c1c(C)cc(=O)[nH]c1C2=O]

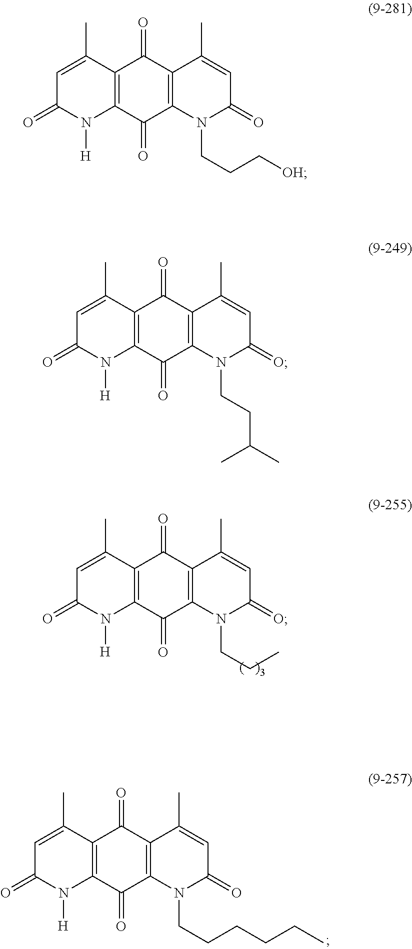

or a salt or solvate thereof. The NQO1 bioactivatable drug may be DNQ or DNQ-87.

The NQO1 bioactivatable drug may be administered before the checkpoint inhibitor, after the checkpoint inhibitor, or concurrent with the checkpoint inhibitor. The NQO1 bioactivatable drug may be administered more than once. The checkpoint inhibitor may be administered more than once. Both the NQO1 bioactivatable drug and the checkpoint inhibitor may be administered more than once.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by particular embodiments and optional features, modification and variation of the concepts herein disclosed may be resorted to by those skilled in the art, and that such modifications and variations are considered to be within the scope of this disclosure as defined by the appended claims.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIGS. 1A-J. NQO1 bioactivatable drug β-lap kills murine tumor cells in an NQO1-dependent manner in vitro and in vivo. (FIG. 1A) NQO1 positive tumor cell lines MC38, TC-1 and Ag104Ld and NQO1 negative cell lines Panc02 and B16 grown in 48-well plates were treated with β-lap (0-8 μM) for a 3 hr followed by washing and replacing medium. Cell viability was determined by Sulforhodamine B (SRB) Assay 4 days later. (FIG. 1B) MC38, TC-1 and Ag104Ld cells were exposed to 4 μM β-lap±dicoumarol (DIC, 50 μM) for 3 hr and cell survival was assessed 4 days later. (FIG. 1C) MC38 cells with CRISPR-based NQO1 Knockout (MC38 NQO1KO #5) planted in 96-well plates were exposed to β-lap for 3 hr and cell survival was assessed 48 hr later. (FIG. 1D) B16 cells stalely harboring a pCMV-NQO1 expression vector (B16 NQO1 #1) planted in 96 well plates were exposed to β-lap±dicoumarol (DIC, 50 μM) for 3 hr and survival assessed 48 hr later. (FIG. 1E) MC38 cells were exposed to a lethal dose of β-lap (4 μM) for indicated times, then stained with 7-AAD and Annexin V followed by flow cytometry analysis. (F-G) MC38 cells (FIG. 1F) or B16 and B16 NQO1 #1 cells (FIG. 1G) were exposed to β-lap±dicoumarol (DIC, 50 μM) for 3 hr and then ROS level was determined by DCFDA cellular ROS assay. (FIG. 1H) MC38 and B16 NQO1 #1 cells were exposed to β-lap for a 3 hr. Catalase (1000 U/ml) was added and cell survival was assessed 48 hr later. (FIG. 1I) C57BL/6 mice (n=5/group) were transplanted with MC38 cells and treated with β-lap (0.03 mg, 0.1 mg or 0.3 mg, intratumorally; or 25 mg/kg, i.v.) every other day for four times. (FIG. 1J) C57BL/6 mice were transplanted with MC38 cells (NQO1 WT or KO, n=5/group) and treated with β-lap (0.3 mg, i.t.) every other day for four times. Tumor growth was monitored twice a week. Data are shown as mean±SEM from two to three independent experiments. **P<0.01, ***P<0.001, ****P<0.0001 determined by unpaired student t-test (FIG. 1F, FIG. 1G and FIG. 1H) or two-way ANOVA (FIG. 1I and FIG. 1J).

FIGS. 2A-F. β-Lap's antitumor effect is CD8⁺ T cell dependent. (FIG. 2A) MC38 cells were subcutaneously transplanted into C57BL/6 WT (n=5-6/group) and Rag1 KO mice (n=5/group), respectively. Tumor bearing mice were treated with β-lap (0.3 mg, i.t.) every other day for four times. Numbers of tumor free mice after treatment were shown. (FIG. 2B) MC38 tumor bearing C57BL/6 mice (n=5/group) were treated with β-lap (0.3 mg, i.t.) every other day for four times. For CD8⁺ T cell depletion, 200 μg of anti-CD8 antibodies were intraperitoneally injected four times at three days interval during the treatment. (FIG. 2C) TC-1 tumor bearing C57BL/6 mice (n=4-5/group) were treated with β-lap (0.1 mg, i.t.) for four times with or without anti-CD8 antibodies. (FIG. 2D) Naïve (n=5/group) and β-lap cured MC38 tumor free (n=7/group) C57BL/6 mice were rechallenged subcutaneously with 3×10⁶ MC38 cells on the opposite site from the primary tumor 30 days after complete rejection, and tumor growth curve was monitored. (FIG. 2E) 2×10⁶ A549 cells were subcutaneously injected into C57BL/6 Rag1^{-/-} mice (n=5/group for vehicle and β-lap; n=7/group for vehicle+OT-1 and β-lap+OT-1). 30 days later, the mice were i.v. adoptively transfected with 2×10⁶ lymph node cells from OT-1 transgenic mice. On the following day, tumor-bearing mice were intratumorally treated with β-lap (0.2 mg) every other day for 4 times. (FIG. 2F) A549 cells were subcutaneously injected into NSG-SGM3 (n=5/group) or NSG-SGM3 harboring human CD34⁺ hematopoietic stem cells (n=5/group for Hu-NSG vehicle; n=6/group for Hu-NSG β-lap). Tumor bearing mice were treated with β-lap (0.2 mg, i.t.) every other day for four times. Tumor growth was measured twice a week. Data are shown as mean±SEM from three independent experiments. **P<0.01, ***P<0.001, ****P<0.0001 determined by two-way ANOVA.

FIGS. 3A-E. Batf3-dependent dendritic cell-mediated T cell cross-priming is required for the antitumor effect of β-lap. (FIG. 3A) MC38 tumor bearing C57BL/6 mice (n=5/group) were treated with β-lap (0.3 mg, i.t.) every other day for three times, and 10 days after the first treatment, lymphocytes from the spleens were isolated and stimulated with medium or MC38 cells irradiated with 60 Gy. (FIG. 3B) MC38-OVA tumor bearing mice (n=4/group) were treated with β-lap (0.3 mg, i.t.) every other day for three times, and 10 days after the first treatment, lymphocytes from the spleens were isolated and stimulated with 2.5 μg/ml of OT-1 peptide. IFNγ producing cells were determined by ELISPOT assay. (FIG. 3C) MC38 tumor bearing C57BL/6 mice (n=5/group) were treated with β-lap (0.3 mg, i.t.) every other day for four times. 100 μg of anti-CSF1R Ab were intratumorally injected three times at three days interval during the treatment. (FIG. 3D) MC38 cells were subcutaneously transplanted into C57BL/6 WT (n=5/group) and Batf3^{-/-} mice (n=5/group for Batf3^{-/-} vehicle; n=6/group for Batf3^{-/-} β-lap), respectively. Tumor bearing mice were treated with β-lap (0.3 mg, i.t.) every other day for four times. Tumor growth was monitored twice a week. (FIG. 3E) MC38-OVA bearing mice (n=3/group) were treated with β-lap (0.3 mg, i.t.) for one time, and 4 days later, CD11c⁺ dendritic cells were purified from the tumor drain lymph node, and cocultured with CD8 T cells isolated from the spleen of OT-1 transgenic mice. The activity of cross-priming of T cells was determined by the level of cell-secreted IFNγ via Cytometric Bead Array (CBA) mouse IFNγ assay. Data are shown as mean±SEM from three independent experiments. **P<0.01, ***P<0.001, ****P<0.0001 determined by unpaired student t-test (FIGS. 3A, 3B and 3E) or two-way ANOVA (FIGS. 3C and 3D).

FIGS. 4A-F. Type I IFNs and TLR4/MyD88/signaling is required for the antitumor effect of β-lap and tumor specific CTLs. (FIG. 4A) MC38 tumor bearing C57BL/6 mice (n=5/group) were treated with β-lap (0.3 mg, i.t.) every other day for four times. Anti-IFNAR blocking antibodies (150 μg, i.t.) were administrated every four days for three times during the treatment. (FIG. 4B) The MC38 tumor bearing WT (n=5/group) and Ifnar 1^{-/-} (n=4/group) C57BL/6 mice were treated with β-lap (0.3 mg, i.t.) every other day for four times. (FIG. 4C) MC38 tumor bearing WT (n=5/group) and Myd88^{-/-} (n=3/group) C57BL/6 mice were treated with β-lap (0.3 mg, i.t.) every other day for four times. (FIG. 4D) MC38 tumor bearing WT (n=5/group) and Tlr4^{-/-} (n=4/group) C57BL/6 mice were treated with β-lap (0.3 mg, i.t.) every other day for four times). (FIG. 4E) MC38 tumor bearing C57BL/6 mice (n=5/group) were treated with β-lap (0.3 mg, i.t.) every other day for four times. Anti-HMGB1 neutralized antibodies (200 μg, i.p) were administrated every three days for three times during the treatment. Tumor growth was monitored twice a week. (FIG. 4F) MC38 tumor bearing WT (n=4/group) or Tlr4^{-/-} (n=4/group) or MyD88−/− (n=6/group) C57BL/6 mice were treated with β-lap (0.3 mg, i.t.) every other day for four times. Anti-HMGB1 neutralized antibodies (200 μg, i.p) were administrated every three days for three times during the treatment. 12 days after the initial treatment, lymphocytes from the TdLN were isolated and stimulated with MC38 tumor cells irradiated with 60 Gy. IFNγ producing cells were determined by ELISPOTs assay. Data are shown as mean±SEM from two to three independent experiments. *P<0.05, **P<0.01, ***P<0.001, ****P<0.0001 determined by two-way ANOVA.

FIGS. 5A-F. β-Lap treatment-induced HMGB1 release enhances tumor immunogenicity and triggers antitumor T cell immunity in vivo. (FIG. 5A) MC38, TC-1 and B16 (NQO null and overexpression clones) were treated with β-lap for 3 hr followed by washing and replacing medium. The level of HMGB1 released into the culture supernatant was determined by ELISA 24 hr later. (FIG. 5B) The research schema for in vivo cross-presentation of tumor specific antigen from β-lap-induced dying tumor cells in FIG. 5C and FIG. 5D. (FIGS. 5C-D) Live or β-lap-induced dying MC38-OVA cells were subcutaneously inoculated into the flank of WT or Tlr4^{-/-} C57BL/6 (n=3-4/group) mice along with or without anti-HMGB1 antibody. 5 days later, the tumor drain lymph mode cells were collected and re-stimulated with OVA protein, OT-1 peptide or irradiated MC38-OVA cells for 48 hr. IFNγ producing cells were determined by ELISPOTs assay (FIG. 5C), and IFNγ secretion level was quantified by CBA mouse IFNγ assay (FIG. 5D). (FIG. 5E) The research schema for the immunogenic vaccine assay in FIG. 5F. (FIG. 5F) MC38-OVA cells treated with β-lap in vitro were inoculated s.c. along with or without anti-HMGB1 antibody into the flank of C57BL/6 mice (n=5-7/group). After 7 days, mice were rechallanged with live MC38-OVA cells by injection into the contralateral flank. The percentage of rechallanged tumor-free mice was shown. Data are shown as mean±SEM from at least two to three independent experiments. *P<0.05, **P<0.01, and ***P<0.001 determined by two-way ANOVA (FIG. 5C and FIG. 5D) or log-rank test (FIG. 5F).

FIGS. 6A-H. β-Lap eradicates large established and checkpoint blockade refractory tumors by combination with anti-PD-L1 therapy. (FIG. 6A) Treatment schema for local β-lap treatment based combinative therapy in FIG. 6B and FIG. 6C. (FIGS. 6B-C) MC38 tumor cells were s.c. inoculated into the flank of C57BL/6 mice (n=5/group). Mice bearing small tumor (about 50 mm³, B) or advanced tumor (about 150-200 mm³, FIG. 6C) were locally treated with β-lap (0.3 mg, i.t.) for four times with or without anti-PD-L1 based checkpoint blockage. Tumor growth was monitored twice a week, and numbers of tumor free mice after treatment were shown. (FIG. 6D) Treatment schema for systemically β-lap treatment based combinative therapy in FIG. 6E and FIG. 6F. (E) MC38 tumor cells were s.c. inoculated into the flank of C57BL/6 mice (n=5-8/group). Tumor bearing mice (about 50-100 mm³) were systemically treated with β-lap (30 mg/kg, i.p.) for six times with or without anti-PD-L1 based checkpoint blockage. Tumor growth was monitored twice a week, and numbers of tumor free mice after treatment were shown. (FIG. 6F) Survival curve for MC38 tumor bearing mice with combinative treatment in FIG. 6E. (FIG. 6G, FIG. 6H) C57BL/6 mice bearing MC38-OVA tumor (about 150 mm³, n=4/group) were locally treated with β-lap (0.3 mg, i.t.) every other day for four times or anti-PD-L1 (100 μg, i.p.) for three times, alone or combination. 12 days after first treatment, tumor infiltrating CD45⁺ cells and lymphocytes were analyzed by flow cytometry (FIG. 6G), and OT-1 antigen specific T cells in the spleen were determined by IFNγ ELISPOT assay (FIG. 6H). Data are shown as mean±SEM from two to three independent experiments. *P<0.05, **P<0.01, ***P<0.001, ****P<0.0001 determined by two-way ANOVA (FIG. 6B, FIG. 6C, FIG. 6E, FIG. 6G and FIG. 6H) or log-rank test (FIG. 6F).

FIG. 7. Proposed model for activation of antitumor immune responses for improved targeted therapy by NQO1 bioactivatable drugs.

FIGS. 8A-E. β-lap induces tumor-specific ROS and evokes caspase independent programmed necrosis. (FIG. 8A) NQO1 expression in multiple murine tumor lines was immunoblotted as indicated. (FIG. 8B) NQO1⁺ cells (MC38, TC-1, and Ag104Ld) and NQO1⁻ cells (B16 and Pan02) were treated for 3 h with β-lap±DIC (50 μM). Drugs were removed and survival assessed 5 days later. (FIG. 8C) Relative H₂O₂ levels were assessed in various cells treated with β-lap for 3 h at the indicated doses using CellRox-Glo. Values were normalized to DMSO-treated control cells. (FIG. 8D) TC-1 cells exposure to β-lap for 12 h, later PARP-1 and Caspase-3 levels were analyzed by western blotting assay. (FIG. 8E) Photos of TC-1 cells exposure to β-lap (4 μM) for 12 h.

FIGS. 9A-B. The antitumor function of β-lap is dependent on CD8+, but not CD4+, T cells. (FIG. 9A) C57BL/6 WT or rag−/− mice (n=5/group) were inoculated subcutaneously (s.c.) with 6×10⁵ MC38 cells and treated intratumorally (i.t.) with 0.3 mg of β-lap or vehicle on day 9, day 12 and day 15. (FIG. 9B) C57BL/6 mice bearing MC38 tumors were treated with β-lap as indicated above. CD4-depleting (clone GK1.5) or CD8-depleting (clone 2.43) antibodies (200 μg) were administered intraperitoneally twice a week, starting on day 8. Tumor growth was measured twice a week. *P<0.05, **P<0.01, ***P<0.001, ****P<0.0001 determined by two-way ANOVA.

FIGS. 10A-C. β-Lap induces tumor regression dependent on STING-dependent DNA sensing and type I IFNs signaling. (FIG. 10A) C57BL/6 WT mice (n=5/group) were inoculated subcutaneously (s.c.) with 6×10⁵ MC38 cells and treated intratumorally (i.t.) with 0.1 mg of β-lap or vehicle on day 14, day 16, day 18 and day 20. Anti-IFNAR blocking Ab (150 μg) were administered i.p. twice a week, starting on day 13. (FIGS. 10B-C) C57BL/6 WT IFNAR^{-/-} or STING^{mut/mut} mice (n=5/group) were inoculated subcutaneously (s.c.) with MC-38 cells and treated intratumorally (i.t.) with 0.3 mg of β-lap or vehicle on day 9, day 12, and day 15. *P<0.05, **P<0.01, ***P<0.001, ****P<0.0001 determined by two-way ANOVA.

FIGS. 11A-D. β-Lap-induced neutrophil infiltration contributes to the antitumor immune response. (FIGS. 11A-B) Single cells were conducted from MC38 (FIG. 11A) or TC-1 (FIG. 11B) tumor tissues after 3 days of β-lap treatment. The frequency of CD11b+ Gr1+ cells were then analyzed by flow cytometry. (FIGS. 11C-D) C57BL/6 WT mice (n=5/group) were inoculated subcutaneously (s.c.) with 6×10⁵ MC38 (FIG. 11C) or 1×10⁵ TC-1 (FIG. 11D) cells and treated intratumorally (i.t.) with 0.3 (mg) of β-lap or vehicle on day 9, day 12, and day 15. Anti-Ly6G blocking Ab (200 μg) were intraperitoneally administered twice a week, starting on day 8. Tumor growth was monitored twice a week. Data are shown as mean±SEM from two to three independent experiments. **P<0.01, ***P<0.001, ****P<0.0001 determined by two-way ANOVA.

FIGS. 12A-B. Low dose of β-Lap synergizes with immune checkpoint blockade (anti-PD-L1) therapy. (FIG. 12A) C57BL/6J WT mice (n=5/group) were inoculated subcutaneously (s.c.) with 6×10⁵ MC38 cells and treated intratumorally (i.t.) with vehicle or 0.1 mg of (β-Lap with or without anti-PD-L1 (Atezolizumab) every three days for 4 injections. Treatment was started when tumor volume was >100 mm³. 100 μg of anti-PD-L1 (clone 10F.9G2) or isotype control antibody (clone LTF-2) were injected intraperitoneally (i.p.) one day before (β-Lap treatment. (FIG. 12B) The same MC38 tumor bearing mice were treated intraperitoneally (i.p.) with vehicle or 30 mg/kg of 13-Lap with or without anti-PD-L1 (Atezolizumab) every three days for 6 injections. Treatment was started when tumor volume was >50 mm³. Tumor diameters were measured by calipers twice a week. Tumor volume is presented as mean±SD. *p<0.05, **p<0.01, ***P<0.001.

FIGS. 13A-B. Radiosensitization of subcutaneous murine cancers by low dose of β-lap. (FIG. 13A) C57BL/6J WT mice (n=5/group) were inoculated subcutaneously (s.c.) with 6×10⁵ MC38 cells and treated intratumorally (i.t.) with vehicle, IR (10 Gy) or 0.1 mg of β-lap with or without IR every three days for 3 injections. Treatment was started when tumor volume was >100 mm³. (FIG. 13B) The same MC38 tumor bearing mice were treated intraperitoneally (i.p.) with vehicle IR (10 Gy) or 30 mg/kg of β-Lap with or without IR every other day for 6 injections. Treatment was started when tumor volume was >50 mm³. IR was given before β-lap treatment. Tumor diameters were measured by calipers twice a week. Tumor volume is presented as mean±SD. *p<0.05, **p<0.01, ***P<0.001.

FIGS. 14A-H. IB-DNQ kills murine cancer cells in an NQO1-dependent manner and induces NAD+/ATP depletion and DNA damage. (FIGS. 14A-C) NQO1 cells MC38 (A) and TC-1 (B) and NQO1⁻ cells B16 (C) were treated for 2 h with various doses of IB-DNQ±DIC (50 μM). Drugs were removed and survival assessed 6 days later. (FIG. 14D) NQO1 expression in multiple murine tumor lines was immunoblotted as indicated. (FIGS. 14E-F) Relative NAD⁺ (E) or ATP (F) levels were assessed in TC-1 cells exposed to various doses of IB-DNQ for 2 h. Values were normalized to DMSO-treated control cells. (FIGS. 14G-H) TC-1 cells exposure to IB-DNQ (0.25 μM) for 60 min, cells were assessed for: total DNA lesions using alkaline comet assays (FIG. 14G). Comet tail lengths in a.u. were monitored at indicated time; and DSBs quantified by γH2AX foci/nuclei using immunofluorescence at indicated time (FIG. 14H). Graphed are means±SD from three experiments in FIGS. 14A-C, FIG. 14E and FIG. 14F. Student's t tests were performed. *p<0.05, **p<0.01, ***P<0.001.

FIGS. 15A-B. IB-DNQ induces tumor regression dependent on the adaptive immune system. (FIG. 15A) C57BL/6J WT or rag−/− mice (n=5/group) were inoculated subcutaneously (s.c.) with 6×10⁵ MC38 cells and treated intratumorally (i.t.) with 0.15 mg of IB-DNQ or vehicle on day 10, day 12, day 14 and day 16. (FIG. 15B) C57BL/6J WT or rag−/− mice (n=5/group) were inoculated subcutaneously (s.c.) with 1×10⁵ TC-1 cells and treated intratumorally (i.t.) with 0.15 mg of IB-DNQ or vehicle on day 10, day 12, day 14 and day 16. Tumor diameters were measured by calipers twice a week. Tumor volume is presented as mean±SD. *p<0.05, **p<0.01, ***P<0.001.

FIGS. 16A-B. IB-DNQ synergizes with immune checkpoint blockade (anti-PD-L1) therapy. C57BL/6J WT mice (n=5/group) were inoculated subcutaneously (s.c.) with 6×10⁵ MC38 cells and treated intratumorally (i.t.) with vehicle, anti-PD-L1 (Atezolizumab) or 0.05 mg of IB-DNQ with or without anti-PD-L1 on day 10, day 13, day 16 and day 19. 100 μg of anti-PD-L1 (clone 10F.9G2) or isotype control antibody (clone LTF-2) were injected intraperitoneally (i.p.) every three days, starting on day 9. Tumor diameters were measured by calipers twice a week. Mice were sacrificed when tumor volume reached to 1000 mm³. (FIG. 16A) Representative mouse tumor volume (mean±SD). (FIG. 16B) Kaplan-Meier survival curves. *p<0.05, **p<0.01, ***P<0.001.

FIGS. 17A-I (related to FIGS. 1A-J). NQO1 bioactivatable drug β-lap kills murine tumor cells in an NQO1-dependent manner in vitro and in vivo. (FIG. 17A) NQO1 expression in different murine cancer cell lines was determined by western blotting assay. (FIG. 17B) NQO1 expression in different clones of MC38 cells with CRISPR-based NQO1 Knockout was determined by western blotting assay. (FIG. 17C) MC38 cells (NQO1 WT or KO) were treated with β-lap for 3 hr followed by washing and replacing fresh medium. Cell viability was determined by Sulforhodamine B (SRB) Assay 48 hr later. (FIG. 17D) NQO1 expression in different clones of B16 cells stably harboring a pCMV-NQO1 expression vector was determined by western blotting assay. (FIG. 17E, FIG. 17F) B16 cells (NQO1 null or stable overexpression) were treated with β-lap with or without dicoumarol (DIC, 50 μM) for 3 hr followed by washing and replacing medium. Cell viability was determined by SRB Assay 48 hr later. (FIG. 17G) NQO1 overexpressing B16 cells (clone #3 and #4) were exposed to β-lap for 3 hr. Catalase (1000 U/ml) was added and cell viability was assessed 48 hr later. (FIG. 17H) C57BL/6 mice (n=5/group) were transplanted with TC-1 cells, and intratumorally treated with β-lap (0.1 or 0.3 mg) every other day for four times. Tumor growth was monitored twice a week. (FIG. 17I) C57BL/6 mice (n=4/group) were transplanted with parental B16 cells (NQO1 null) or NQO1 stable overexpressing B16 cells (clone #1 and #4) and treated with β-lap (0.3 mg, i.t.) every other day for four times. Data are shown as mean±SEM. **P<0.01, ***P<0.001, ****P<0.0001 determined by unpaired student t-test (FIG. 17G) or two-way ANOVA (FIG. 17H and FIG. 17I).

FIGS. 18A-D (to FIGS. 2A-F). β-Lap-mediated antitumor effect depends on immune-mediated killing. (FIG. 18A) B16 NQO1 #1 cells were subcutaneously transplanted into C57BL/6 WT and Rag1KO mice (n=5/group), respectively. Tumor bearing mice were treated with β-lap (0.3 mg, i.t.) every other day for four times. (FIG. 18B) Changes of the immune cells in the tumor microenvironment 7 days after β-lap treatment. C57BL/6 mice (n=4/group) were transplanted with MC38 cells and intratumorally treated with 0.3 mg of β-lap for two times. 7 days after the last treatment, tumor tissue was removed and digested, and immune cells were analyzed with flow cytometer (unpaired student t-test was used to analyze the significance of changes, *P<0.05, **P<0.01). (FIG. 18C) C57BL/6 mice were transplanted with MC38 cells and treated with β-lap (0.3 mg, i.t.) every other day for four times. For T cell depletion, 200 μg of anti-CD4 or anti-CD8 antibodies were injected four times at three days interval during the treatment. (FIG. 18D) NQO1 positive human lung carcinoma lines A549 grown in 48-well plates were exposed to β-lap (0-6 μM)±dicoumarol (DIC, 50 μM) for 3 hr and cell survival was assessed 4 days later with SRB Assay. Data are shown as mean±SEM.

FIGS. 19A-C (related to FIGS. 4A-F). TLR4/MyD88 pathway but not TLR9 signaling is required for the antitumor effect of β-lap. (FIGS. 19A-B) MC38-OVA bearing mice were treated with β-lap (0.3 mg, i.t.) for two times. Six days later, tumor tissues were collected for both single cell digestion (FIG. 19A) and RNA extraction (FIG. 19B).

(FIG. 19A) The secreted IFNβ from the suspended cell supernatant was measured by ELISA after a 24 hr culture. (FIG. 19B) The mRNA levels of IFNα1, IFNγ, TNFα and CXCL10 were determined by real-time PCR assay. (FIG. 19C) MC38 cells were implanted into Myd88^{-/-}, Tlr4^{-/-} and Tlr9^{-/-} C57BL/6 mice, respectively. Tumor bearing mice were then treated with β-lap (0.3 mg, i.t.) every other day for four times. Tumor growth was monitored twice a week. Data are shown as mean±SEM. *P<0.05, **P<0.01, ***P<0.001 determined by unpaired t-test.

FIGS. 20A-F (related to FIGS. 6A-H). Both local and systemic β-lap treatment can synergize with anti-PD-L1 immune checkpoint blockage. (FIG. 20A) MC38 tumor cells were s.c. inoculated into the flank of C57BL/6 mice (n=5/group). Mice bearing advanced tumor (about 150-200 mm³) were locally treated with β-lap (0.3 mg, i.t.) for four times with or without anti-PD-L1 based checkpoint blockade (100 μg, i.p.) for three times. Tumor growth was monitored twice a week, and the growth curve of the individual mouse in each group was shown. (FIG. 20B) MC38 tumor cells were s.c. inoculated into the flank of C57BL/6 mice (n=5/group). Mice bearing advanced tumor (about 100 mm³) were locally treated with low dose of β-lap (0.1 mg, i.t.) for four times with or without PD-L1 based checkpoint blockage. (FIG. 20C, FIG. 20D) MC38 tumor bearing mice (about 50-100 mm³, n=5-8/group) were systematically treated with β-lap (30 mg/kg, i.p.) for six times with or without PD-L1 based checkpoint blockage. Tumor growth (FIG. 20C) and body weight (FIG. 20D) were monitored twice a week. Growth curve of the individual mouse in each group was shown. (FIG. 20E, FIG. 20F) B16 cells with stable NQO1 overexpression (Mixed clone #1, #3 and #4) were s.c. inoculated into C57BL/6 mice. Tumor bearing mice (about 100 mm³) were locally treated with β-lap (0.3 mg, i.t.) for four times with or without anti-PD-L1 based checkpoint blockage (150 μg, i.p.) for three times. Treatment schema was shown (FIG. 20E) and the tumor growth curve was monitored (FIG. 20F). Data are shown as Mean±SEM from at least two independent experiments. *P<0.05, **P<0.01, ***P<0.001, ****P<0.0001 determined by two-way ANOVA.

FIGS. 21A-F. IB-DNQ selectively induces NQO1⁺ tumor cell death via intensive tumor-specific ROS production and extensive DNA damage. (FIG. 21A) Viability of murine cell lines following IB-DNQ treatment for 4 h. TC-1, Ag104Ld and MC38 cells express endogenous NQO1, while Pan02 and B16 cells are null for NQO1. (FIG. 21B) Viability of NQO1 KO (MC38 NQO1^{-/-}) and NQO1 overexpression (B16 NQO1⁺) cells following IB-DNQ treatment for 4 h. (FIG. 21C) ROS levels in MC38 cells after a 1 h exposure to IB-DNQ. (FIGS. 21D-E) DNA double strand break was assessed via comet assay (FIG. 21D) and γH2AX foci/nuclei immunofluorescence (FIG. 21E). (FIG. 21F) MC38 cell death after a 4 h exposure to IB-DNQ was determined by 7AAD and Annexin V staining using flow cytometry analysis. (FIG. 21A), (FIG. 21B) Data are shown as mean±SD from three independent experiments (six replicates/each experiment); (FIGS. 21C-F) Data are shown as mean±SD from three independent experiments. Statistical analysis was performed using an unpaired Student's 2-tailed t test. *P<0.05; **P<0.01; ***P<0.001; NS, not significant.

FIGS. 22A-F. IB-DNQ-mediated antitumor effect involves the immune system. 6×10⁵ MC38 or 8×10⁵ MC38 NQO1 KO cells were subcutaneously transplanted into Rag1^{-/-}, C57BL/6, and NSG mice, respectively (n=5/group). Tumor-bearing mice were treated with IB-DNQ (12 mg/kg, i.t.) or 20% HPβCD (Vehicle) every other day for four times after tumor volumes reached to 50 mm³. Tumor volumes and survival were assessed. (FIG. 22A) Tumor volumes of MC38 in WT C57BL/6 and NSG mice. (FIG. 22B) Survival curves of MC38 tumor-bearing WT C57BL/6 and NSG mice. (FIGS. 22C-D) Tumor volumes of MC38 and TC-1 in WT C57BL/6 and Rag1^{-/-} mice, respectively. (FIGS. 22E-F) Tumor volumes and survival analysis of MC38 and MC38 NQO1 KO models in C57BL/6 mice. Data are shown as mean±SD from at least two independent experiments. Statistical analysis was performed using an unpaired Student's 2-tailed t test. *P<0.05; **P<0.01; ***P<0.001; NS, not significant.

FIGS. 23A-D. IB-DNQ-mediated antitumor effect influences tumor microenvironment. 6×10⁵ MC38 cells were subcutaneously transplanted into C57BL/6 mice (n=3/group). Tumor-bearing mice were treated with IB-DNQ (12 mg/kg, i.t.) or vehicle every other day for four times after tumor volumes reached to 50 mm³. Tumors were collected 24 h later after the last IB-DNQ injection. (FIG. 23A) Flow cytometry analysis of tumor-infiltrating immune cells from vehicle and IB-DNQ treated tumors. (FIG. 23B) Quantification of immune populations from vehicle and IB-DNQ treated tumors. (FIG. 23C) T cell proliferation in the presence/absence of IB-DNQ. CFSE labeled splenocytes were treated with lethal dose IB-DNQ for 4 h followed by washing and stimulation with anti-CD3 (1 μg/ml) and anti-CD28 (2 μg/ml) for 48 h, proliferative CD8 T cells were then analyzed by flow cytometry assay. (FIG. 23D) Effect of IB-DNQ on T cells. Splenocytes from naïve (n=3/group) and tumor-bearing (n=3/group) mice were exposed to different concentrations of IB-DNQ for 4 h followed by washing and replacing the medium. 24 h later, cell death was analyzed by flow cytometry. Ag104 cells with the same treatment were used as control. Data are shown as mean±SD from two independent experiments. Statistical analysis was performed using an unpaired Student's 2-tailed t test. *P<0.05; **P<0.01; NS, not significant.

FIGS. 24A-F. CD8⁺ and CD4⁺ T cells are critical for the IB-DNQ-mediated antitumor effect. 6×10⁵ MC38 cells were subcutaneously transplanted into C57BL/6 mice (n=5/group). Tumor-bearing mice were treated with IB-DNQ (12 mg/kg, i.t.) or vehicle every other day for four times after tumor volumes reached to 50 mm³. For CD4⁺ and/or CD8⁺ T cell depletion, 200 μg of anti-CD4 and/or CD8⁺ antibodies were intraperitoneally injected three times at three-day intervals during the treatment. (FIGS. 24A-B) Tumor volumes and survival analysis of MC38-bearing mice treated with/without IB-DNQ±anti-CD4 antibodies. (FIGS. 24C-D) Tumor volumes and survival analysis of MC38-bearing mice treated with/without IB-DNQ±anti-CD8 antibodies. (FIGS. 24E-F) Tumor volumes and survival analysis of MC38-bearing mice treated with/without IB-DNQ±anti-CD4 and anti-CD8 antibodies. Data are shown as mean±SD from at least two independent experiments. Statistical analysis was performed using an unpaired Student's 2-tailed t test. *P<0.05; **P<0.01; ***P<0.001; NS, not significant.

FIGS. 25A-E. IB-DNQ induces tumor ICD and dendritic cell-mediated T cell cross-priming. (FIG. 25A) The levels of HMGB1 released into cell culture supernatant after IB-DNQ treatment. MC38, MC38 NOQ1^{-/-}, B16, and B16 NOQ1⁺ cells were treated with IB-DNQ for 4 h followed by replacing medium and growth for 24 h, culture supernatant was collected and HMGB1 levels were determined by ELISA. (FIGS. 25B-C) Relative expression of IFN-α and IFN-β. Tumor samples were the same ones as FIG. 23A, total RNA extractions were performed according to manufacturer's instructions, after reverse transcription into cDNA, qPCR was carried out. (FIGS. 25D-E) IFN-γ-indicated T cell response. 6×10⁵ MC38 or 8×10⁵ MC38-OVA cells were transplanted into C57BL/6 mice (n=3/group). After tumors reached to 50 mm³, mice were treated with IB-DNQ or vehicle every other day for four times, and 24 h later after the last administration, splenocytes isolated from tumor-bearing mice were stimulated with medium or MC38 cells irradiated with 60 Gy (FIG. 25D) or OT-1 (FIG. 25E). Data are shown as mean±SD from at least two independent experiments. Statistical analysis was performed using an unpaired Student's 2-tailed t test. *P<0.05; **P<0.01; ***P<0.001.

FIGS. 26A-H. IB-DNQ induces innate immune memory instead of classical immunological memory. Naïve (n=5/group) and IB-DNQ-cured MC38 tumor-free (n=7/group) C57BL/6 mice were re-challenged subcutaneously with 3×10⁶ MC38 cells on the opposite site from the primary tumor 60 days after complete rejection. Organs from these tumor-free mice were collected 30 days after tumor eradicated again. (FIGS. 26A-B) Tumor volumes and survival analysis of MC38 model. (FIGS. 26C-D) Memory CD8⁺ T cells (FIG. 26C) and CD4⁺ T cells (FIG. 26D) in different organs. (FIG. 26E) CD44⁺ DCs in lymph nodes (LN). (FIG. 26F) DCs from LNs of tumor-free (TF) or tumor-bearing mice were stimulated with antigen induced by IB-DNQ (1 μM) for 5 h, then CD44 expression on DCs was assessed. (FIG. 26G) CD8⁺ T cells separated from spleen of TF or tumor-bearing mice were labeled with CFSE and co-cultured with antigen induced by IB-DNQ (1 μM) for 48 h. T cell proliferation was determined by flow cytometry. (FIG. 26H) CFSE labeled splenocytes were co-cultured with cells from LN of TF or tumor-bearing mice in the presence of antigen, anti-CD3 (1 μg/ml), and anti-CD28 (2 μg/ml) stimulation for 48 h, T cell proliferation was determined by flow cytometry. Data are shown as means±SD from three independent experiments. Statistical analysis was performed using an unpaired Student's 2-tailed t test. *P<0.05; **P<0.01; ***P<0.001; NS, not significant.

FIGS. 27A-B. PD-L1 upregulation in THE after IB-DNQ treatment. 6×10⁵ MC38 cells were subcutaneously transplanted into C57BL/6 mice (n=5/group). Tumor-bearing mice were treated with IB-DNQ (12 mg/kg, i.t.) or vehicle every other day for four times after tumor volumes reached to 50 mm³ (Small) or 150 mm³ (Big). (FIGS. 27A-B) Small tumors (FIG. 27A) or advanced tumors (FIG. 27B) were collected 24 h later after the last IB-DNQ injection. PD-L1 protein levels in CD45⁺ immune cells or CD45⁻ tumor cells were measured by flow cytometry. Statistical analysis was performed using an unpaired Student's 2-tailed t test. *P<0.05; NS, not significant.

FIGS. 28A-F. Combination therapy of IB-DNQ with anti-PD-L1 overcomes checkpoint blockade resistance. (FIGS. 28A-D) Mice bearing small (50 mm³, red line) or advanced (150 mm³, blue line) MC38 tumors were treated with IB-DNQ (12 mg/kg, i.v.) or vehicle every other day for 4 injections (FIGS. 28A-B), or treated with mIgG or anti-PD-L1 for three injections (FIGS. 28C-D). (FIGS. 28E-F) MC38 tumor-bearing mice (150 mm³) were treated with IB-DNQ, anti-PD-L1 or IB-DNQ+ anti-PD-L1 every other day for 4 injections, Vehicle was mIgG or HPβCD. Tumor volumes (FIGS. 28A, 28C and 28E) and overall survival (FIGS. 28B, 28D and 28F) were measured. Tumor growth was measured twice a week. Data are shown as mean±SD from three independent experiments. The Kaplan-Meier survival curve was defined for overall survival by Prism 8 software. *P<0.05, **P<0.01, ***P<0.001 determined by two-way ANOVA. NS, not significant.

### DETAILED DESCRIPTION

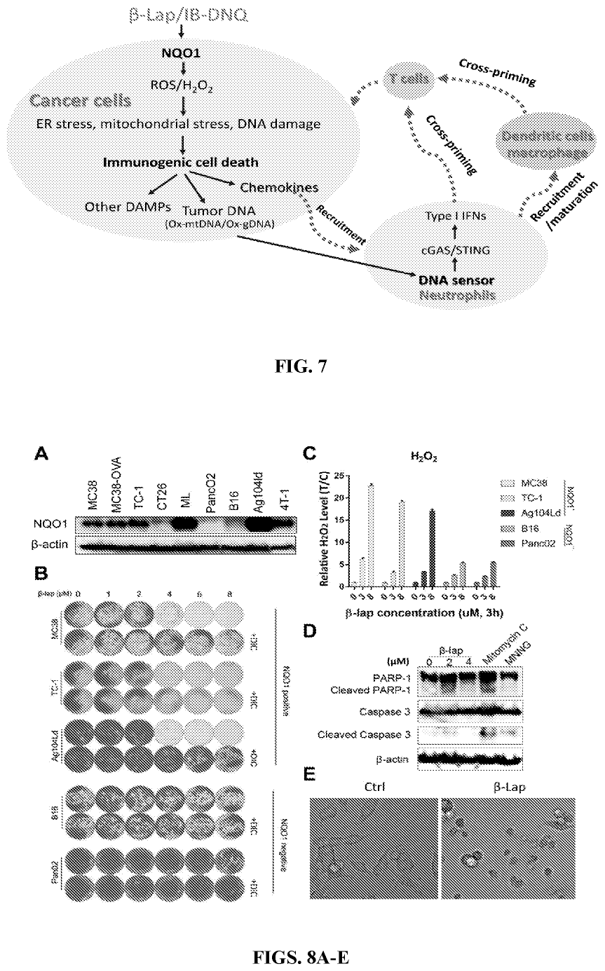

The inventors' previous studies revealed that NQO1 bioactivatable drugs (β-lap and IB-DNQ) can lead to extensive DNA damage and PARP1-driven tumor programmed necrosis, while simultaneously inducing tumor suppression in immunodeficient mice. Strategies to increase the efficacy of sublethal doses of these NQO1 bioactivatable drugs are being sought to increase their efficaciousness within vigorous chemotherapeutic or radiotherapeutic regimen. Here, the inventors show that both neutrophil-mediated innate immunity and CD8-mediated adaptive immunity systems are stimulated, leading to more efficacious antitumor effects of NQO1 bioactivatable drugs in immunocompetent mice. They have also revealed that NQO1 bioactivatable drugs can trigger immunogenic cell death (ICD) and induce damage-associated molecular patterns (DAMPs) release, and phagocytes/APCs (antigen-presenting cells) recruitment, which in turn promote cross-priming of cytotoxic T cells (CTLs) for suppression of tumor growth through increasing antigen/DNA uptake and type I interferons (IFNs) production (FIG. 7). This study shows how tumor-specific reactive oxygen species (ROS) and DNA damage induced by NQO1 bioactivatable drugs can stimulate antitumor immunity, and whether activation of these responses can improve tumor targeted therapy efficacy. The combination of NQO1 bioactivatable drugs with an approach that activates adaptive immunity, such as T cell checkpoint blockade, will provide durable efficacy and patient benefit. These and other aspects of the disclosure are set out in detail below.

### I. DEFINITIONS

In general, the terms and phrases used herein have their art-recognized meaning, which can be found by reference to standard texts, journal references and contexts known to those skilled in the art. Such art-recognized meanings may be obtained by reference to technical dictionaries, such as Hawley's Condensed Chemical Dictionary 14^{th} Edition, by R. J. Lewis, John Wiley & Sons, New York, N.Y., 2001.

BER, base excision repair; SSBR, single strand break repair; DSBR, double strand break repair.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

While the present invention can take many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications of the described embodiments and any further applications of the principles of the invention as described herein are contemplated as would normally occur to one skilled in the art to which the invention relates.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Whenever a range is given in the specification, for example, a temperature range, a time range, a carbon chain range, or a composition or concentration range, all intermediate ranges and subranges, as well as all individual values included in the ranges given are intended to be individually included in the disclosure. It will be understood that any subranges or individual values in a range or subrange that are included in the description can be optionally excluded from embodiments of the invention.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein, any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

A “chemotherapeutic agent” refers to any substance capable of reducing or preventing the growth, proliferation, or spread of a cancer cell, a population of cancer cells, tumor, or other malignant tissue. The term is intended also to encompass any antitumor or anticancer agent.

A “therapeutically effective amount” of a compound with respect to the subject method of treatment refers to an amount of the compound(s) in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate. The term “treating” or “treatment” can include reversing, reducing, or arresting the symptoms, clinical signs, and underlying pathology of a condition in manner to improve or stabilize a subject's condition.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “exposing” is intended to encompass definitions as broadly understood in the art. In an embodiment, the term means to subject or allow to be subjected to an action, influence, or condition. For example, and by way of example only, a cell can be subjected to the action, influence, or condition of a therapeutically effective amount of a pharmaceutically acceptable form of a chemotherapeutic agent.

The term “cancer cell” is intended to encompass definitions as broadly understood in the art. In an embodiment, the term refers to an abnormally regulated cell that can contribute to a clinical condition of cancer in a human or animal. In an embodiment, the term can refer to a cultured cell line or a cell within or derived from a human or animal body. A cancer cell can be of a wide variety of differentiated cell, tissue, or organ types as is understood in the art.

The term “tumor” refers to a neoplasm, typically a mass that includes a plurality of aggregated malignant cells.

The following groups can be R groups or bridging groups, as appropriate, in the formulas described herein.

The term “alkyl” refers to a monoradical branched or unbranched saturated hydrocarbon chain preferably having from 1 to 30 carbon atoms. Short alkyl groups are those having 1 to 12 carbon atoms including methyl, ethyl, propyl, butyl, pentyl, and hexyl groups, including all isomers thereof. Long alkyl groups are those having 12-30 carbon atoms. The group may be a terminal group or a bridging group.

Alkyl, heteroalkyl, aryl, heteroaryl, and heterocycle groups, and cyclic and/or unsaturated versions thereof, can be R groups of Formula I, and each group can be optionally substituted.

The term “substituted” indicates that one or more hydrogen atoms on the group indicated in the expression using “substituted” is replaced with a “substituent”. The number referred to by ‘one or more’ can be apparent from the moiety one which the substituents reside. For example, one or more can refer to, e.g., 1, 2, 3, 4, 5, or 6; in some embodiments 1, 2, or 3; and in other embodiments 1 or 2. The substituent can be one of a selection of indicated groups, or it can be a suitable group known to those of skill in the art, provided that the substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, aroyl, (aryl)alkyl (e.g., benzyl or phenylethyl), heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl (alkyl)amine, and cyano. Additionally, suitable substituent groups can be, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, —NC(═O)R, —C(═O)R, —C(═O)NRR, —S(═O)₂O⁻, —S(═O)₂OH, —S(═O)₂R, —OS(═O)₂OR, —S(═O)₂NR, —S(═O)R, —OP(═O)O₂RR, —P(═O)O₂RR, —P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, or —C(NR)NRR, where each X is independently a halogen (“halo”): F, Cl, Br, or I; and each R is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above can be excluded from the group of potential values for substituents on a substituted group.

The term “heteroalkyl,” by itself or in combination with another term, means, unless otherwise stated, a stable straight or branched chain, or cyclic hydrocarbon radical, or combinations thereof, often having from 2 to 14 carbons, or 2 to 10 carbons in the chain, including at least one carbon atom and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. The heteroalkyl group can have, for example, one to about 20 carbon atoms in a chain. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Additional examples of heteroalkyl groups include alkyl ethers, secondary and tertiary alkyl amines, amides, alkyl sulfides, and the like. The group may be a terminal group or a bridging group. As used herein, reference to a chain when used in the context of a bridging group refers to the direct chain of atoms linking the two terminal positions of the bridging group.

The term “alcohol” as used herein may be defined as an alcohol that comprises a C₁₋₁₂ alkyl moiety substituted at a hydrogen atom with one hydroxyl group. Alcohols include ethanol, n-propanol, i-propanol, n-butanol, i-butanol, s-butanol, t-butanol, n-pentanol, i-pentanol, n-hexanol, cyclohexanol, n-heptanol, n-octanol, n-nonanol, n-decanol, and the like. The carbon atoms in alcohols can be straight, branched or cyclic.

“Acyl” may be defined as an alkyl-CO— group in which the alkyl group is as described herein. Examples of acyl include acetyl and benzoyl. The alkyl group can be a C₁-C₆ alkyl group. The group may be a terminal group or a bridging (i.e., divalent) group.

“Alkoxy” refers to an —O-alkyl group in which alkyl is defined herein. Preferably the alkoxy is a C₁-C₆alkoxy. Examples include, but are not limited to, methoxy and ethoxy. The group may be a terminal group or a bridging group.

“Alkenyl” as a group or part of a group denotes an aliphatic hydrocarbon group containing at least one carbon-carbon double bond and which may be straight or branched preferably having 2-14 carbon atoms, more preferably 2-12 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkynyl” as a group or part of a group may be defined as an aliphatic hydrocarbon group containing a carbon-carbon triple bond, the chain of which may be straight or branched preferably having from 2-14 carbon atoms, more preferably 2-12 carbon atoms, more preferably 2-6 carbon atoms in the normal chain. Exemplary structures include, but are not limited to, ethynyl and propynyl. The group may be a terminal group or a bridging group.

“Alkenyloxy” refers to an —O— alkenyl group in which alkenyl is as defined herein. Preferred alkenyloxy groups are C₁-C₆ alkenyloxy groups. The group may be a terminal group or a bridging group.

“Alkynyloxy” refers to an —O-alkynyl group in which alkynyl is as defined herein. Preferred alkynyloxy groups are C₁-C₆ alkynyloxy groups. The group may be a terminal group or a bridging group.

“Alkoxycarbonyl” refers to an —C(O)—O-alkyl group in which alkyl is as defined herein. The alkyl group is preferably a C₁-C₆ alkyl group. Examples include, but not limited to, methoxycarbonyl and ethoxycarbonyl. The group may be a terminal group or a bridging group.

“Alkylsulfinyl” may be defined as a —S(O)-alkyl group in which alkyl is as defined above. The alkyl group is preferably a C₁-C₆ alkyl group. Exemplary alkylsulfinyl groups include, but not limited to, methylsulfinyl and ethylsulfinyl. The group may be a terminal group or a bridging group.

“Alkylsulfonyl” refers to a —S(O)₂-alkyl group in which alkyl is as defined above. The alkyl group is preferably a C₁-C₆ alkyl group. Examples include, but not limited to methylsulfonyl and ethylsulfonyl. The group may be a terminal group or a bridging group.

“Amino” refers to —NH₂, and “alkylamino” refers to —NR₂, wherein at least one R is alkyl and the second R is alkyl or hydrogen. The term “acylamino” refers to RC(═O)NH—, wherein R is alkyl or aryl. The alkyl group can be, for example, a C₁-C₆ alkyl group. Examples include but are not limited to methylamino and ethylamino. The group may be a terminal group or a bridging group.

“Alkylaminocarbonyl” refers to an alkylamino-carbonyl group in which alkylamino is as defined above. The group may be a terminal group or a bridging group.

“Cycloalkyl” refers to a saturated or partially saturated, monocyclic or fused or spiro polycyclic, carbocycle of 3 to about 30 carbon atoms, often containing 3 to about 9 carbons per ring, such as cyclopropyl, cyclobutyl, cyclopentyl, cyclohexyl, cyclooctyl, and the like. It includes monocyclic systems such as cyclopropyl and cyclohexyl, bicyclic systems such as decalin, and polycyclic systems such as adamantane. The group may be a terminal group or a bridging group.

“Cycloalkenyl” may be defined as a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

Alkyl and cycloalkyl groups can be substituents on the alkyl portions of other groups, such as without limitation, alkoxy, alkyl amines, alkyl ketones, arylalkyl, heteroarylalkyl, alkylsulfonyl and alkyl ester substituents and the like. The group may be a terminal group or a bridging group.

“Cycloalkylalkyl” may be defined as a cycloalkyl-alkyl-group in which the cycloalkyl and alkyl moieties are as previously described. Exemplary monocycloalkylalkyl groups include cyclopropylmethyl, cyclopentylmethyl, cyclohexylmethyl and cycloheptylmethyl. The group may be a terminal group or a bridging group.

“Heterocycloalkyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

“Heterocycloalkenyl” refers to a heterocycloalkyl as described above but containing at least one double bond. The group may be a terminal group or a bridging group.

“Heterocycloalkylalkyl” refers to a heterocycloalkyl-alkyl group in which the heterocycloalkyl and alkyl moieties are as previously described. Exemplary heterocycloalkylalkyl groups include (2-tetrahydrofuryl)methyl and (2-tetrahydrothiofuranyl)methyl. The group may be a terminal group or a bridging group.

“Halo” refers to a halogen substituent such as fluoro, chloro, bromo, or iodo.

The term “aryl” refers to an aromatic hydrocarbon group derived from the removal of one hydrogen atom from a single carbon atom of a parent aromatic ring system. The radical can be at a saturated or unsaturated carbon atom of the parent ring system. The aryl group can have from 6 to 18 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted, as described above for alkyl groups.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, as described above in the definition of “substituted”. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In another embodiment heteroaryl denotes an ortho-fused bicyclic heterocycle of about eight to ten ring atoms derived therefrom, particularly a benz-derivative or one derived by fusing a propylene, trimethylene, or tetramethylene diradical thereto.

The term “heterocycle” refers to a saturated or partially unsaturated ring system, containing at least one heteroatom selected from the group oxygen, nitrogen, and sulfur, and optionally substituted with one or more groups as defined herein under the term “substituted”. A heterocycle can be a monocyclic, bicyclic, or tricyclic group containing one or more heteroatoms. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The abbreviation “DNQ_{d}” as used herein refers to an analog or derivative of DNQ.

Additional groups that can be bridging groups or terminal groups of R₁, R₂, R₃, and R₄ are described below.

The term “carbonate ester” may be defined as a functional group having a general structure R′OC(═O)OR, where R′ can be the tricyclic core of Formula I and R can be as defined in the definitions of the variables of Formula I.

The term “ester” may be defined as a functional group having a general structure RC(═O)OR′, where R′ can be the tricyclic core of Formula I and R can be as defined in the definitions of the variables of Formula I, or vice versa.

A “pyridyl” group can be a 2-pyridyl, 3-pyridyl, or 4-pyridyl group.

The term “sulfhydryl” may be defined as a functional group having a general structure —S—H.

The term “sulfinyl” may be defined as a functional group having a general structure R—S(═O)—R′, where R′ can be the tricyclic core of Formula I and R can be as defined in the definitions of the variables of Formula I, or vice versa.

The term “sulfonyl” may be defined as a functional group having a general structure R—S(═O)₂—R′, where R′ can be the tricyclic core of Formula I and R can be as defined in the definitions of the variables of Formula I, or vice versa.

The term “hexose” may be defined as a monosaccharide having six carbon atoms having the general chemical formula C₆H₁₂O₆ and can include aldohexoses which have an aldehyde functional group at position 1 or ketohexoses which have a ketone functional group at position 2. Example aldohexoses include, allose, altrose, glucose, mannose, gulose, idose, galactose, and talose, in either D or L form.

Abbreviations used in the Schemes and Examples may include the following:
A549=adenocarcinomic human alveolar basal epithelial cells
ATP=adenosine triphosphate
β-lap=β-lapachone
DHE=dihydroethidium
DNQ=Deoxynyboquinone
DNQ_{d}=Any analog or derivative of deoxynyboquinone
ELISA=enzyme-linked immunosorbent assay
h=hour(s)
H596=[NCI-H596] human lung adenosquamous carcinoma cell line
HT1080=primate fibrosarcoma cell line
LD₅₀=lethal dose having 50% probability of causing death
LD₉₀=lethal dose having 90% probability of causing death
LD₁₀₀=lethal dose having 100% probability of causing death
MCF-7=human breast adenocarcinoma cell line
MDA-MB-231=human breast cancer cell line
MIA-PaCa2=Pancreatic cancer cell line
mins=minute(s)
NADH=nicotinamide adenine dinucleotide
NQO1=NAD(P)H:quinone oxidoreductase 1
NSCLC=non-small-cell lung cancer cells
OCR=oxygen consumption rates
p53=a tumor suppressor protein
PC-3=human prostate cancer cell line
ROS=reactive oxygen species
±SE=standard error
siRNA=small interfering ribonucleic acid
shRNA=small hairpin ribonucleic acid
μM=micromolar
nM=nanomolar
μmol=micromole

### II. Therapeutic Quinones

DNQ is a potent chemotherapeutic agent exhibiting a wide therapeutic window that holds great promise for targeted therapy against a wide spectrum of difficult to treat cancers, including pancreatic and non-small cell lung cancer. Despite considerable advances in cancer chemotherapy, the lack of selectivity of most cancer chemotherapeutics remains a major limiting factor. Elevated NAD(P)H:quinone oxidoreductase-1 (NQO1, DT-diaphorase, EC 1.6.99.2) levels found in most solid tumors, particularly in non-small-cell lung cancer cells (NSCLC), prostate, pancreatic and breast, provide a target for therapeutic treatments described herein. NQO1 is an inducible Phase II detoxifying two-electron oxidoreductase capable of reducing most quinones, forming stable hydroquinones. In most cases, glutathione transferase then detoxifies hydroquinones, conjugating them with glutathione for secretion, and effectively avoiding more toxic semiquinones.

For some rare compounds, however, NQO1-mediated bioreduction can be exploited for antitumor activity. Rather than promoting detoxification, NQO1 activity can convert specific quinones into highly cytotoxic species. Most antitumor quinones dependent on NQO1 are DNA alkylators: (a) mitomycin C (MMC); (b) RH1; (c) E09; and (d) AZQ. However, these DNA alkylators are not only subject to detoxification pathways, but resistance from elevated or inducible DNA repair pathways limit their usefulness. Furthermore, many of these drugs are efficient substrates for one-electron oxidoreductases ubiquitously expressed in normal tissues.

The ortho-naphthoquinone, β-lapachone (β-lap, Scheme 1), kills cultured cancer cells and murine xenograft and orthotopic human or mouse tumor models in vivo in an NQO1-dependent manner. In contrast to alkylating quinones, β-lap induces cell death by NQO1-dependent reactive oxygen species (ROS) formation and oxidative stress. NQO1 metabolism of β-lap results into an unstable hydroquinone that is spontaneously oxidized by two equivalents of dioxygen, generating superoxide.

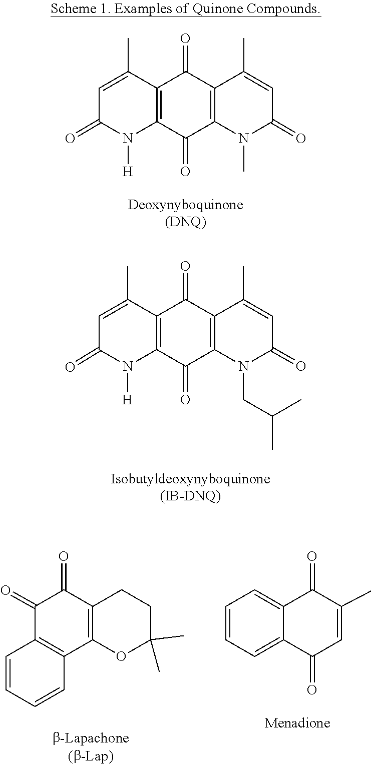

A futile cycle of oxidoreduction is thus established, and elevated superoxide levels, in turn cause massive DNA base and single strand break (SSBs) lesions that normally are easily and rapidly repaired. However, extensive DNA lesions created in β-lap-treated NQO1 overexpressing cancer cells results in hyperactivation of poly(ADP-ribose)polymerase-1 (PARP1), an otherwise essential base and SSB repair enzyme. In turn, PARP1 hyperactivation results in dramatic reduction of the NAD⁺/ATP pool due to ADP-ribosylation, causing tremendous energy depletion and cell death. As a result, β-lap kills NQO1+ cancer cells by a unique programmed necrosis mechanism that is: (a) independent of caspase activation or p53 status; (b) independent of bcl-2 levels; (c) not affected by BAX/BAK deficiencies; (d) independent of EGFR, Ras or other constitutive signal transduction activation; and/or (e) not dependent on proliferation, since NQO1 is expressed in all cell cycle phases. Thus, β-lap is an attractive experimental chemotherapeutic, and various β-lap formulations have been, or are in, phase I/II clinical trials.

Deoxynyboquinone (DNQ, Scheme 1) is a promising anti-neoplastic agent. Prior data indicated that DNQ kills cancer cells through oxidative stress and ROS formation. The cytotoxicity of DNQ was partially prevented by N-acetylcysteine, a global free radical scavenger and precursor to glutathione. It has now been shown that DNQ undergoes an NQO1-dependent futile cycle similar to β-lap, where oxygen is consumed, and ROS is formed and extensive DNA damage triggers PARP1 hyperactivation, with dramatic decreases in essential NAD⁺/ATP nucleotide pools, indicative of programmed necrosis. Importantly, DNQ is 20- to 100-fold more potent than β-lap, with a significantly enhanced therapeutic window in NQO1+ versus NQO1-NSCLC cells. Efficacious NQO1-dependent killing by DNQ is also shown in breast, prostate, and pancreatic cancer models in vitro. Furthermore, in vitro NQO1 processes DNQ much more efficiently than β-lap, indicating that increased utilization accounts for its increased potency. Thus, DNQ offers significant promise as a selective chemotherapeutic agent for the treatment of solid tumors with elevated NQO1 levels, however, the combination therapy described herein can provide efficacious therapies with a variety of quinone compounds due to the synergy of the combination.

Because NQO1 is overexpressed in the majority of solid tumors, and the cytotoxicity of the various quinone compounds depends predominately on the elevated expression of the enzyme NQO1, thus the quinine compounds and their derivatives can be excellent means to approach targeting solid tumors. The invention provides numerous new cytotoxic compounds that can be used as new cancer therapeutics, as described herein.

The foregoing and other objects and features of the disclosure will become more apparent from the following detailed description, which proceeds with reference to the accompanying figures. Further embodiments, forms, features, aspects, benefits, objects, and advantages of the present application shall become apparent from the detailed description and figures provided herewith.

NQO1 bioactivatable drugs (all β-lapachone and DNQ derivatives that are substrates for NQO1) generate tremendous levels of reactive oxygen species in an NQO1-dependent, tumor-selective manner, allowing the use of DNA repair inhibitors, including all PARP1 inhibitors, DNA double strand break repair inhibitors, as well as base excision repair inhibitors, to be used in a tumor-specific manner, effecting a tumor-selective efficacy of both agents. DNA repair inhibitors, in general, have failed because of the lack of tumor selectivity. Because these NQO1 bioactivatable drugs cause tumor selective production of DNA lesions, including DNA base damage, single strand breaks and double strand breaks, DNA repair inhibitors can be used to provide tumor selective antitumor activity. Tumor-selective activity and responses include dramatic inhibition of glycolysis as well as other tumor-selective metabolism inhibition.

NQO1 bioactivatable drugs can be used to make DNA repair inhibitors tumor-selective in a manner that is not obvious unless one knows the DNA lesions generated, and in a manner that causes metabolic changes and cell death responses that are not obvious and are altered depending on the DNA repair inhibitors used. For example, PARP1 inhibitors administered with DNQ bioactivatable drugs cause standard apoptotic responses without energy losses. In contrast, DNA double strand break repair, single strand break repair, and base excision repair inhibitors enhance PARP1 hyperactivation, with subsequent losses in energy metabolism and programmed necrosis.

The only current use of DNA repair inhibitors, such as PARP-1 inhibitors, is through the unique exploitation of tumor-specific synthetic lethality responses (e.g., use of P ARP1 inhibitors in BRACA1/2 mutant tumors). This, however, is a very limited use of DNA repair inhibitors—approximately only 5% of breast cancers only. In contrast, the approach described herein can treat all cancers having elevated NQO1 and lowered Catalase levels, while normal tissue have elevated Catalase and low levels of NQO1. The methods described herein provide a new use of DNA repair inhibitors, allowing for their use in a tumor-selective manner, while also potentiating NQO1 bioactivatable drugs. Both agents can be used at nontoxic doses to render synergistic, tumor-selective efficacy responses.

To date, DNA repair inhibitors fail in the lack of tumor-selective responses and efficacy. The methods described herein resolve these limitations, while greatly potentiating NQO1 bioactivatable drugs. The methods also allow the use of 2- to >4-fold lower doses of NQO1 bioactivatable drugs, resolving toxic effects of these NQO1 bioactivatable drugs (e.g., methemaglobinemia). In the therapeutic methods, the inhibitors can be added before and after NQO1 bioactivatable drugs, at a minimum before, and optionally before, simultaneously, after, or a combination thereof. Cell death responses depend on inhibitor used. The responses are not obvious and would entail specific biomarkers to follow in vivo.

The disclosure provides DNQ compounds, beta-lapachone and derivatives thereof, and the use of NQO1 bioactivatable drugs in combination therapy for the treatment of cancer. Examples of DNQ compounds include compounds of Formula (I):

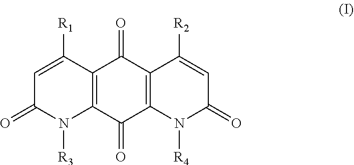

wherein

R₁, R₂, R₃, and R₄ are each independently —H or —X—R;

each X is independently a direct bond or a bridging group, wherein the bridging group is —O—, —S—, —NH—, —C(═O)—, —O—C(═O)—, —C(═O)—O—, —O—C(═O)—O—, or a linker of the formula —W-A-W—, wherein

each W is independently —N(R′)C(═O)—, —C(═O)N(R)—, —OC(═O)—, —C(═O)O—, —O—, —S—, —S(O)—, —S(O)₂—, —N(R′)—, —C(═O)—, —(CH₂)ₙ— where n is 1-10, or a direct bond, wherein each R′ is independently H, (C₁-C₆)alkyl, or a nitrogen protecting group; and each A is independently (C₁-C₂₀)alkyl, (C₂-C₁₆)alkenyl, (C₂-C₁₆)alkynyl, (C₃-C₈)cycloalkyl, (C₆-C₁₀)aryl, —(OCH₂—CH₂)ₙ— where n is 1 to about 20, —C(O)NH(CH₂)ₙ— wherein n is 1 to about 6, —OP(O)(OH)O—, —OP(O)(OH)O(CH₂)ₙ— wherein n is 1 to about 6, or (C₁-C₂₀)alkyl, (C₂-C₁₆)alkenyl, (C₂-C₁₆)alkynyl, or —(OCH₂—CH₂)ₙ— interrupted between two carbons, or between a carbon and an oxygen, with a cycloalkyl, heterocycle, or aryl group;

each R is independently alkyl, alkenyl, alkynyl, heteroalkyl, cycloalkyl, cycloalkenyl, heterocycloalkyl, heterocycloalkenyl, (cycloalkyl)alkyl, (heterocycloalkyl)alkyl, (cycloalkyl)heteroalkyl, (heterocycloalkyl)heteroalkyl, aryl, heteroaryl, (aryl)alkyl, (heteroaryl)alkyl, hydrogen, hydroxy, hydroxyalkyl, alkoxy, (alkoxy)alkyl, alkenyloxy, alkynyloxy, (cycloalkyl)alkoxy, heterocycloalkyloxy, amino, alkylamino, aminoalkyl, acylamino, arylamino, sulfonylamino, sulfinylamino, —COR^{x}, —COOR^{x}, —CONHR^{x}, —NHCOR^{x}, —NHCOOR^{x}, —NHCONHR^{x}, —N₃, —CN, —NC, —NCO, —NO₂, —SH, -halo, alkoxycarbonyl, alkylaminocarbonyl, sulfonate, sulfonic acid, alkylsulfonyl, alkylsulfinyl, arylsulfonyl, arylsulfinyl, aminosulfonyl, R^{x}S(O)R^{y}—, R^{x}S(O)₂R^{y}—, R^{x}C(O)N(R^{x})R^{y}—, R^{x}SO₂N(R^{x})R^{y}—, R^{x}N(R^{x})C(O)R^{y}—, R^{x}N(R^{x})SO₂R^{y}—, R^{x}N(R^{x})C(O)N(R^{x})R^{y}—, carboxaldehyde, acyl, acyloxy, —OPO₃H₂, —OPO₃Z₂ where Z is an inorganic cation, or saccharide; where each R^{x} is independently H, OH, alkyl or aryl, and each R^{y} is independently a group W; wherein any alkyl or aryl can be optionally substituted with one or more hydroxy, amino, cyano, nitro, or halo groups; or a salt or solvate thereof.

In some embodiments, when R₁, R₂, and R₃ are methyl, R₄ is not H or methyl. In other embodiments, when R₁, R₃, and R₄ are methyl, the group —X—R of R₂ is not —CH₂—OAc. In certain embodiments, when R₁, R₃, and R₄ are methyl, the R group of R₂ is not acyloxy. In various embodiments, R₁-R₄ are not each H. In certain embodiments, R₁-R₄ are not each alkyl, such as unsubstituted alkyl. In some embodiments, R₁-R₄ are not each methyl.

In one embodiment, R₁, R₂, R₃, and R₄ are each (C₁₋₂₀)alkyl groups. In some embodiments, the (C₁₋₂₀)alkyl group is a (C₂₋₂₀)alkyl group, a (C₃₋₂₀)alkyl group, a (C₄₋₂₀)alkyl group, a (C₅₋₂₀)alkyl group, or a (C₁₀₋₂₀)alkyl group. The alkyl groups can be substituted, for example, with a hydroxyl or phosphate group. The phosphate group can be a phosphonic acid or a phosphonic acid salt, such as a lithium salt, a sodium salt, a potassium salt, or other known salt of phosphonic acids.

A specific value for R₁ is H. A specific value for R₂ is H. A specific value for R₃ is H. A specific value for R₄ is H.

A specific value for R₁ is methyl. A specific value for R₂ is methyl. A specific value for R₃ is methyl. A specific value for R₄ is methyl. The methyl can be substituted as described above for the term “substituted”.

In some embodiments of Formula (I):
R₁ and R₂ are methyl; R₃ is hydrogen; and R₄ is 2-methyl-propane;
R₁ and R₂ are methyl; R₃ is hydrogen; and R₄ is butyl;
R₁ and R₄ are methyl and R₃ is hydrogen; and R₂ is ethyl;
R₁ and R₂ are methyl and R₃ is hydrogen; and R₄ is ethyl;
R₁ is methyl; R₃ is hydrogen; R₂ is propyl; and R₄ is butyl;
R₁ and R₄ are methyl; R₂ is propyl and R₃ is hydrogen;
R₁ is propyl; R₂ and R₄ are methyl and R₃ is hydrogen;
R₁ and R₂ are ethyl; R₃ is hydrogen; and R₂ is methyl;
R₁ is propyl; R₂ is methyl; R₃ is hydrogen; and R₄ is butyl;
R₁ and R₂ are propyl; R₃ is hydrogen; and R₄ is butyl;
R₁ and R₂ are methyl; R₃ is hydrogen; and R₄ is C₁₂alkyl;
R₁ and R₂ are methyl; R₃ is hydrogen; and R₄ is tert-butyl;
R₁ and R₂ are methyl; R₃ is hydrogen; and R₄ is hydroxypropyl;
R₁ and R₂ are methyl; R₃ is hydrogen; and R₄ is 3,3-dimethylbutyl[—CH₂CH₂C(CH₃)₂CH₃];
R₁ and R₂ are methyl; R₃ is hydrogen; and R₄ is 3-methybutyl[—CH₂CH₂CH(CH₃)CH₃];
R₂ and R₄ are methyl; R₃ is hydrogen; and R₁ is ethyl;
R₁ and R₂ are methyl; R₃ is hydrogen; and R₄ is propyl;
R₁ and R₂ are methyl; R₃ is hydrogen; and R₄ is n-pentyl;
R₁ and R₂ are methyl; R₃ is hydrogen; and R₄ is n-hexyl;
R₁ and R₂ are methyl; R₃ is hydrogen; and R₄ is isopropyl;
R₁ and R₂ are methyl; R₃ is hydrogen; and R₄ is cyclooctyl;
R₁ and R₂ are methyl; R₃ is hydrogen; and R₄ is cyclopropyl;
R₁ and R₂ are methyl; R₃ is hydrogen; and R₄ is methylcyclopropyl;
R₁ and R₂ are methyl; R₃ is hydrogen; and R₄ is ethylcyclopropyl;
R₁ is C₁₂alkyl; R₂ and R₄ are methyl; and R₃ is hydrogen;
R₁ and R₄ are methyl; R₃ is hydrogen; and R₂ is C₁₂alkyl;
R₁, R₂, and R₃ are methyl; and R₄ is —CH₂OPO₃Na₂;
R₁ is —CH₂OPO₃Na₂; R₂ and R₃ are methyl; and R₄ is hydrogen;
R₁ and R₃ are methyl; R₂ is —CH₂OPO₃Na₂; and R₄ is hydrogen;
R₁ and R₂ are methyl; R₃ is —CH₂OPO₃Na₂; and R₄ is hydrogen;
R₁ and R₂ are methyl; R₃ is —CH₂CH₂OPO₃Na₂; and R₄ is hydrogen;
R₁, R₂, and R₃ are methyl; and R₄ is —CH₂OH;
R₁ is —CH₂OH; R₂ and R₃ are methyl; and R₄ is hydrogen;
R₁ and R₃ are methyl; R₂ is —CH₂OH; and R₄ is hydrogen;
R₁ and R₂ are methyl; R₃ is —CH₂OH; and R₄ is hydrogen; or
R₁ and R₂ are methyl; R₃ is —CH₂CH₂OH; and R₄ is hydrogen.

In certain embodiments of Formula I, R¹ is (C₁₋₄) alkyl group. In certain instances, R¹ is (C₁₋₃) alkyl group. In certain instances, R¹ is (C₁₋₂) alkyl group.

In certain embodiments of Formula I, R² is (C₁₋₄) alkyl group. In certain instances, R² is (C₁₋₃) alkyl group. In certain instances, R² is (C₁₋₂) alkyl group.

In certain embodiments of Formula I, R³ is hydrogen.

In certain embodiments of Formula I, R⁴ is an optionally substituted (C₁₋₁₀) alkyl group, where the alkyl group is substituted with hydroxyl, halogen, amino, or thiol. In certain instances, R⁴ is (C₁₋₁₀) alkyl group, (C₁₋₈) alkyl group, (C₁₋₆) alkyl group, or (C₁₋₄) alkyl group. In certain instances, R⁴ is (C₂-6) alkyl group. In certain instances, R⁴ is a substituted (C₁₋₁₀) alkyl group, substituted (C₁₋₈) alkyl group, substituted (C₁₋₆) alkyl group, or substituted (C₁₋₄) alkyl group, where the alkyl group is substituted with hydroxyl, halogen, amino, or thiol. In certain instances, R⁴ is an alkyl group is substituted with hydroxyl. In certain instances, R⁴ is an alkyl group is substituted with halogen. In certain instances, R⁴ is an alkyl group is substituted with amino. In certain instances, R⁴ is an alkyl group is substituted with thiol.

In certain embodiments of Formula I, R¹ and R² are independently (C₁₋₄) alkyl groups; R³ is hydrogen; and R⁴ is an optionally substituted (C₁₋₁₀) alkyl group, where the alkyl group is substituted with hydroxyl, halogen, amino, and thiol.

In certain embodiments of Formula I, R¹ and R² are independently (C₁₋₂) alkyl groups; R³ is hydrogen; and R⁴ is an optionally substituted (C₁₋₁₀) alkyl group, where the alkyl group is substituted with hydroxyl, halogen, amino, and thiol.

In certain embodiments of Formula I, R¹ and R² are independently (C₁₋₂) alkyl groups; R³ is hydrogen; and R⁴ is (C₁₋₁₀) alkyl group. In certain embodiments of Formula I, R¹ and R² are independently (C₁₋₂) alkyl groups; R³ is hydrogen; and R⁴ is (C₁₋₈) alkyl group. In certain embodiments of Formula I, R¹ and R² are independently (C₁₋₂) alkyl groups; R³ is hydrogen; and R⁴ is (C₁₋₆) alkyl group. In certain embodiments of Formula I, R¹ and R² are independently (C₁₋₂) alkyl groups; R³ is hydrogen; and R⁴ is (C₁₋₄) alkyl group. In certain embodiments of Formula I, R¹ and R² are independently (C₁₋₂) alkyl groups; R³ is hydrogen; and R⁴ is (C₂-6) alkyl group. In certain embodiments of Formula I, R¹ and R² are independently (C₁₋₂) alkyl groups; R³ is hydrogen; and R⁴ is a substituted (C₁₋₆) alkyl group, where the alkyl group is substituted with hydroxyl, halogen, amino, and thiol. In certain embodiments of Formula I, R¹ and R² are independently (C₁₋₂) alkyl groups; R³ is hydrogen; and R⁴ is a substituted (C₁₋₄) alkyl group, where the alkyl group is substituted with hydroxyl, halogen, amino, and thiol.

In certain embodiments, a compound of Formula I is Compound 87 or a salt or solvate thereof:

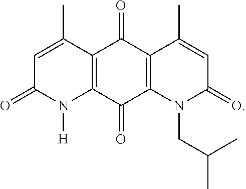 [SMILES: Cc1cc(=O)[nH]c2c1C(=O)c1c(C)cc(=O)n(CC(C)C)c1C2=O]

In certain embodiments, a compound of Formula I is Compound 9-253 or a salt or solvate thereof:

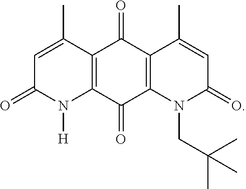 [SMILES: Cc1cc(=O)[nH]c2c1C(=O)c1c(C)cc(=O)n(CC(C)(C)C)c1C2=O]

In certain embodiments, a compound of Formula I is Compound 9-251 or a salt or solvate thereof:

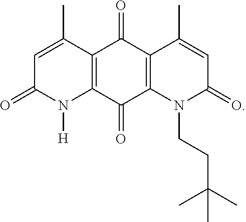 [SMILES: Cc1cc(=O)[nH]c2c1C(=O)c1c(C)cc(=O)n(CCC(C)(C)C)c1C2=O]

In certain embodiments, a compound of Formula I is Compound 10-41 or a salt or solvate thereof:

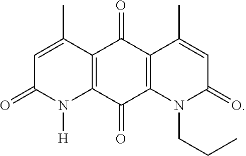 [SMILES: CCCn1c2c(c(C)cc1=O)C(=O)c1c(C)cc(=O)[nH]c1C2=O]

In certain embodiments, a compound of Formula I is Compound 109 or a salt or solvate thereof:

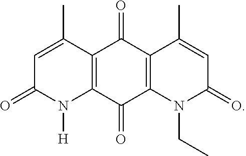 [SMILES: CCn1c2c(c(C)cc1=O)C(=O)c1c(C)cc(=O)[nH]c1C2=O]

In certain embodiments, a compound of Formula I is Compound 107 or a salt or solvate thereof:

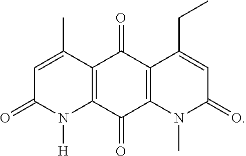 [SMILES: CCc1cc(=O)n(C)c2c1C(=O)c1c(C)cc(=O)[nH]c1C2=O]

In certain embodiments, a compound of Formula I is Compound 9-281 or a salt or solvate thereof:

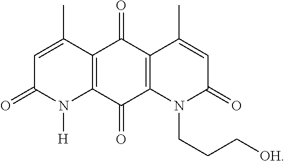 [SMILES: Cc1cc(=O)[nH]c2c1C(=O)c1c(C)cc(=O)n(CCCO)c1C2=O]

In certain embodiments, a compound of Formula I is Compound 9-249 or a salt or solvate thereof:

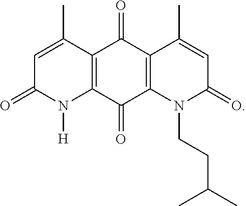 [SMILES: Cc1cc(=O)[nH]c2c1C(=O)c1c(C)cc(=O)n(CCC(C)C)c1C2=O]

In certain embodiments, a compound of Formula I is Compound 9-255 or a salt or solvate thereof:

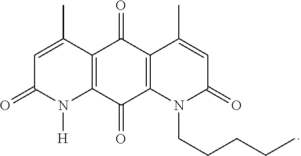 [SMILES: CCCCCn1c2c(c(C)cc1=O)C(=O)c1c(C)cc(=O)[nH]c1C2=O]

In certain embodiments, a compound of Formula I is Compound 9-257 or a salt or solvate thereof:

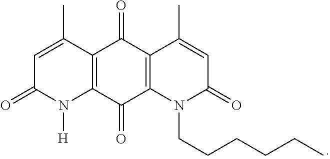 [SMILES: CCCCCCn1c2c(c(C)cc1=O)C(=O)c1c(C)cc(=O)[nH]c1C2=O]

The disclosure also provides a pharmaceutical composition comprising a compound of Formula (I) and a pharmaceutically acceptable diluent, excipient, or carrier. The carrier can be water, for example, in the presence of hydroxypropyl-β-cyclodextrin (HPβCD). The solubility of the compound can be increase by about 100 times, about 200 times, about 500 times, about 1000 times, about 2000 times, or about 3000 times, compared to the compound's solubility in water without HPβCD. Additional DNQ compounds and methods are described by International Application No. PCT/US12/59988 (Hergenrother et al.).

As to any of the above formulas or groups that contain one or more substituents, it is understood, of course, that such groups do not contain any substitution or substitution patterns that are sterically impractical and/or synthetically non-feasible. In addition, the compounds of this disclosure include all stereochemical isomers arising from the substitution of these compounds.

Selected substituents of the compounds described herein may be present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosure. One of ordinary skill in the art of medicinal and organic chemistry understands the versatility of such substituents. To the degree that recursive substituents are present in a claim of the disclosure, the total number will be determined as set forth above. In some embodiments, recursive substituents are present only to the extent that the molecular mass of the compound is about 400 to about 1600, about 450 to about 1200, about 500 to about 100, about 600 to about 800. In other embodiments, recursive substituents are present only to the extent that the molecular mass of the compound is less than 2000, less than 1800, less than 1600, less than 1500, less than 1400, less than 1200, less than 1000, less than 900, less than 800, less than 750, less than 700, or less than about 600.

Patients with solid tumors having elevated NQO1 levels can be treated through the administration of an effective amount of a pharmaceutically active form of DNQ and/or DNQ_{d} (DNQ compounds). The following scheme shows several more particular formulae:

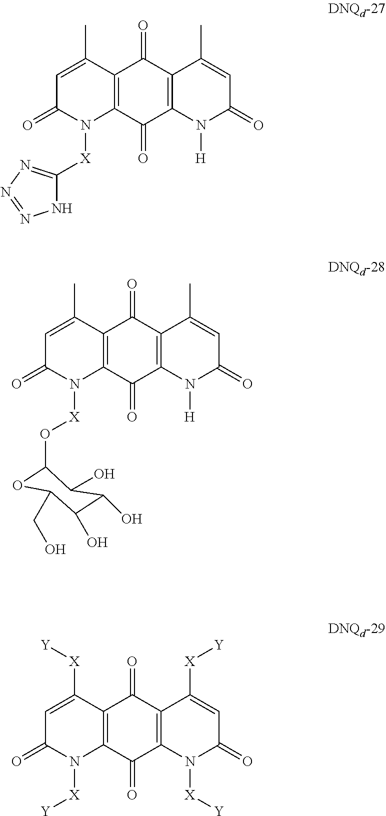

For DNQ_{d}-27 and DNQ_{d}-28, X can be a linker of formula —W-A-W— or a divalent bridging group such as a divalent alkyl, alkenyl, alkynyl, heteroalkyl, acycloalkyl, cycloalkenyl, heterocycloalkyl, heterocycloalkenyl, cycloalkylalkyl, heterocycloalkylalkyl, cycloalkylheteroalkyl, heterocycloalkylheteroalkyl, alkoxy, alkoxyalkyl, alkenyloxy, alkynyloxy, cycloalkylkoxy, heterocycloalkyloxy, amino, alkylamino, aminoalkyl, acylamino, arylamino, sulfonylamino, sulfinylamino, alkoxycarbonyl, alkylaminocarbonyl, sulfonyl, alkylsulfonyl, alkylsulfinyl, arylsulfonyl, arylsulfinyl, aminosulfonyl, or acyl, each of which may be optionally substituted.

For DNQ_{d}-29, each X can independently be a linker of formula —W-A-W— or a divalent bridging group as described above for DNQ_{d}-27 and DNQ_{d}-28; and each Y can independently be:

| | |
|---|---|
| (1) | Hydroxyl, |
| (2) | Aldehyde, |
| (3) | Carboxyl, |
| (4) | Haloformyl, |
| (5) | Hydroperoxy, |
| (6) | Phenyl, |
| (7) | Benzyl, |
| (8) | Alkyl, |
| (9) | Alkenyl, |
| (10) | Alkynyl, |
| (11) | Acetate, |
| (12) | Amino, |
| (13) | Azide, |
| (14) | Azo, |
| (15) | Cyano, |
| (16) | Isocyanato, |
| (17) | Nitrate, |
| (18) | Isonitrile, |
| (19) | Nitrosooxy, |
| (20) | Nitro, |
| (21) | Nitroso, |
| (22) | Pyridyl, |
| (23) | Sulfhydryl, |
| (24) | Sulfonic acid, |
| (25) | Sulfonate, |
| (26) | Isothiocyanato, |
| (27) | Phosphine, |
| (28) | Phosphate, |
| (29) | Halo, or |
| (30) | Hexose. |

Pharmaceutically acceptable salts of compounds described herein are within the scope of the disclosure and include acid or base addition salts which retain the desired pharmacological activity and are not biologically undesirable (e.g., the salt is not unduly toxic, allergenic, or irritating, and is bioavailable). When a compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the disclosure has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Many of the molecules disclosed herein contain one or more ionizable groups (groups from which a proton can be removed (e.g., —COOH) or added (e.g., amines) or which can be quaternized (e.g., amines)). All possible ionic forms of such molecules and salts thereof are intended to be included individually in the disclosure herein. With regard to salts of the compounds described herein, one of ordinary skill in the art can select from among a wide variety of available counterions those that are appropriate for preparation of salts of this disclosure for a given application. In specific applications, the selection of a given anion or cation for preparation of a salt may result in increased or decreased solubility of that salt.

Examples of suitable salts of the compounds described herein include their hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. These salts may be prepared by methods known to those skilled in the art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the disclosure can contain both basic and acidic functionalities that allow the compounds to be converted into either base or acid addition salts.

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by the disclosure and are intended to be within the scope of the disclosure.

The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix. The compounds of the formulas described herein can be hydrates.

### II. METHODS OF MAKING DNQ COMPOUNDS

The invention also relates to methods of making the compounds and compositions of the invention. The compounds and compositions can be prepared by any of the applicable techniques of organic synthesis. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as standard organic reference texts such as March's Advanced Organic Chemistry: Reactions, Mechanisms, and Structure, 5^{th} Ed. by M. B. Smith and J. March (John Wiley & Sons, New York, 2001), Comprehensive Organic Synthesis; Selectivity, Strategy & Efficiency in Modern Organic Chemistry, in 9 Volumes, Barry M. Trost, Ed.-in-Chief (Pergamon Press, New York, 1993 printing)); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999), each of which are hereby incorporated by reference.

A number of exemplary methods for the preparation of the compositions of the disclosure are provided below. These methods are intended to illustrate the nature of such preparations are not intended to limit the scope of applicable methods. Additional methods and useful techniques are described in WO 2013/056073 (Hergenrother et al.).

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically, the temperatures will be −100° C. to 200° C., solvents will be aprotic or protic depending on the conditions required, and reaction times will be 1 minute to 10 days. Work-up typically consists of quenching any unreacted reagents followed by partition between a water/organic layer system (extraction) and separation of the layer containing the product. Oxidation and reduction reactions are typically carried out at temperatures near room temperature (about 20° C.), although for metal hydride reductions frequently the temperature is reduced to 0° C. to −100° C. Heating can also be used when appropriate. Solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions). Standard synthetic techniques such as azeotropic removal of reaction by-products and use of anhydrous reaction conditions (e.g. inert gas environments) are common in the art and will be applied when applicable.

Protecting Groups. The term “protecting group”, “blocking group”, or “PG” refers to any group which, when bound to a hydroxy or other heteroatom prevents undesired reactions from occurring at this group and which can be removed by conventional chemical or enzymatic steps to reestablish the hydroxyl group. The particular removable blocking group employed is not always critical and preferred removable hydroxyl blocking groups include conventional substituents such as, for example, allyl, benzyl, acetyl, chloroacetyl, thiobenzyl, benzylidene, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product. The R groups of Formula (I) can also be protecting groups, as described herein.

Suitable hydroxyl protecting groups are known to those skilled in the art and disclosed in more detail in T. W. Greene, Protecting Groups In Organic Synthesis; Wiley: New York, 1981 (“Greene”) and the references cited therein, and Kocienski, Philip J.; Protecting Groups (Georg Thieme Verlag Stuttgart, New York, 1994), both of which are incorporated herein by reference.

Protecting groups are available, commonly known and used, and are optionally used to prevent side reactions with the protected group during synthetic procedures, i.e. routes or methods to prepare the compounds by the methods of the disclosure. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis.

Protecting groups do not need to be, and generally are not, the same if the compound is substituted with multiple PGs. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered and may occur in any order as determined by the artisan.

Various functional groups of the compounds of the disclosure may be protected. For example, protecting groups for —OH groups (whether hydroxyl, carboxylic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art. For further detail regarding carboxylic acid protecting groups and other protecting groups for acids, see Greene, cited above. Such groups include by way of example and not limitation, esters, amides, hydrazides, and the like.

### III. CHECKPOINT INHIBITORS

Checkpoint inhibitor therapy is a form of cancer treatment immunotherapy currently under research. The therapy targets immune checkpoints, key regulators of the immune system that stimulate or inhibit its actions, which tumors can use to protect themselves from attacks by the immune system. Checkpoint therapy can block inhibitory checkpoints, restoring immune system function. The first anti-cancer drug targeting an immune checkpoint was ipilimumab, a CTLA-4 blocker approved in the United States in 2011.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

In some embodiments, the immune checkpoint inhibitor therapy may be molecules targeting adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), T-cell immunoglobulin domain and mucin domain 3 (TIM-3) and V-domain Ig suppressor of T cell activation (VISTA).

The immune checkpoint inhibitors may be drugs such as small molecules, recombinant forms of ligand or receptors, or, in particular, are antibodies, such as human antibodies (e.g., International Patent Publication No. WO2015016718; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

### IV. PHARMACEUTICAL COMPOSITIONS AND METHODS OF TREATMENT

A. Pharmaceutical Compositions

The following describes information relevant to pharmaceutical and pharmacological embodiments and is further supplemented by information in the art available to one of ordinary skill. The exact formulation, route of administration and dosage can be chosen by an individual physician or clinician in view of a patient's condition (see e.g., Fingl et al., in The Pharmacological Basis of Therapeutics, 1975, Ch. 1).

It should be noted that the attending physician would know how to and when to terminate, interrupt, or adjust administration due to toxicity, or to organ dysfunctions, etc. Conversely, the attending physician would also know to adjust treatment to higher levels if the clinical response were not adequate (in light of or precluding toxicity aspects). The magnitude of an administered dose in the management of the disorder of interest can vary with the severity of the condition to be treated and to the route of administration. The severity of the condition may, for example, be evaluated, in part, by standard prognostic evaluation methods. Further, the dose and perhaps dose frequency, can also vary according to circumstances, e.g., the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995) and elsewhere in the art.

The compounds can be administered to a patient in combination with a pharmaceutically acceptable carrier, diluent, or excipient. The phrase “pharmaceutically acceptable” refers to those ligands, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” includes any and all solvents, dispersion media, diluents, coatings, surfactants, antioxidants, preservatives (e.g., antibacterial agents, antifungal agents), isotonic agents, absorption delaying agents, salts, buffers, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the chemotherapeutic or pharmaceutical compositions is contemplated.

A DNQ_{d} or DNQ compound may be combined with different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present disclosure administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

When administered to a subject, effective amounts will depend, of course, on the particular cancer being treated; the genotype of the specific cancer; the severity of the cancer; individual patient parameters including age, physical condition, size and weight, concurrent treatment, frequency of treatment, and the mode of administration. These factors are well known to the physician and can be addressed with no more than routine experimentation. In some embodiments, it is preferred to use the highest safe dose according to sound medical judgment.

In certain embodiments, pharmaceutical compositions may comprise, for example, at least about 0.1% of a DNQ_{d} or DNQ compound. In other embodiments, the active compound may comprise between about 2% to about 75% of the weight of the unit, or between about 25% to about 60%, for example, and any range derivable therein. In other non-limiting examples, a dose may also comprise from about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more per administration, and any range derivable therein. In non-limiting examples of a derivable range from the numbers listed herein, a range of about 10 mg/kg/body weight to about 100 mg/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

Actives described herein such as DNQ_{d} or DNQ compounds may be formulated into a composition in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, triethylamine, histidine or procaine.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are optionally provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose (HPC); or combinations thereof such methods. In many cases, it will be preferable to include isotonic agents, such as, for example, sugars, sodium chloride or combinations thereof.

Sterile injectable solutions are prepared by incorporating the active compounds in the required amount of the appropriate solvent with various other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose.

The composition should be stable under the conditions of manufacture and storage, and preserved against the contaminating action of microorganisms, such as bacteria and fungi. Thus, preferred compositions have a pH greater than about 5, preferably from about 5 to about 8, more preferably from about 5 to about 7. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

B. Formulation of DNQ Compounds for In Vivo Administration

The aqueous solubility of DNQ at pH 7.4 in phosphate buffered saline (PBS) was measured by LC-MS. DNQ was sonicated for 30 minutes in PBS then undissolved solid was removed by filtration through a 0.45 μm syringe filter and the filtrate was analyzed by LC-MS (λ=275 nm, ESI-TOF in negative mode). The optimal sonication time was determined by sonicating DNQ for 1, 5, 10, and 30 minutes. While the concentration of DNQ in solution increased substantially between 1, 5, and 10 minutes, there was only a minor difference between 10 and 30 minutes. During the 30-minute sonication the water bath warmed to 45° C. (samples were cooled to room temperature before filtration). A calibration curve was generated from 1-100 μM by dissolving DNQ in methanol to a concentration of 500 μM and making dilutions of this stock in 80:20 water:methanol. The calibration curve (measure by UV absorbance) was linear over this range; 1 μM was approximately the limit of detection. The solubility of DNQ in PBS was measured to be 115 μM. The solution was very pale yellow.

Because of the poor aqueous solubility of DNQ, the inventors investigated the use of 2-hydroxypropyl-beta-cyclodextrin (HPβCD), a common excipient, to improve the solubility of DNQ. In the absence of HPβCD, the solubility of DNQ increases significantly in strongly basic solutions and DNQ precipitates when the pH is returned to neutral. However, in the presence of a sufficient amount of HPβCD, DNQ does not precipitate when the pH is returned to neutral. This same neutral solution of DNQ in HPβCD cannot be made directly (i.e. without pH adjustment). This indicates that DNQ compounds deprotonate in base and this deprotonated molecule forms a tight complex with HPβCD which is stable enough to prevent protonation as the pH decreases. The only proton on DNQ that might reasonably be deprotonated in aqueous base is the N—H. Although the acidity of the N—H bond of DNQ has not been measured, it has been measured for a derivative of DNQ and found to have a pKa of 8.0.

The protocol for formulating DNQ compounds in HPβCD is as follows: the DNQ compound is slurried in a 20% solution of HPβCD in pH 7.4 PBS and the pH is then increased by the addition of 10 M NaOH to induce dissolution of the DNQ compound. The pH is returned to pH 7.5-8.0 by the careful addition of 1 M HCl. A 3.3 mM solution of the DNQ compound can be made by this method which is stable at least 24 hours. This represents a 30-fold increase in solubility of DNQ over PBS alone. The inventors initially chose a 20% HPβCD solution. However, the inventors have found that β-lap was formulated as a 40% solution of HPβCD for human clinical trials and the inventors' experience with DNQ indicates that the concentration of DNQ increases linearly with that of HPβCD; thus a 40% HPβCD solution would permit the creation of a 6.6 mM solution of DNQ and other DNQ compounds.

C. Treatments

The disclosure also provides methods of treating a patient that has tumor cells having elevated NQO1 levels. The methods can include administering to a patient having tumor cells with elevated NQO1 levels a therapeutically effective amount of a compound of Formula (I), or a composition described herein. The disclosure further provides methods of treating a tumor cell having an elevated NQO1 level comprising exposing the tumor cell to a therapeutically effective amount of a compound or composition described herein, wherein the tumor cell is treated, killed, or inhibited from growing. The tumor or tumor cells can be malignant tumor cells. In some embodiments, the tumor cells are cancer cells, such as Non-Small-Cell Lung Carcinoma.

The methods of the disclosure may be thus used for the treatment or prevention of various neoplasia disorders including acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor. Accordingly, the compositions and methods described herein can be used to treat bladder cancer, brain cancer (including intracranial neoplasms such as glioma, meninigioma, neurinoma, and adenoma), breast cancer, colon cancer, lung cancer (SCLC or NSCLC) ovarian cancer, pancreatic cancer, and prostate cancer.

D. Combination Therapy

Active ingredients described herein (e.g., compounds of Formula (I)) can also be used in combination with other active ingredients. Such combinations are selected based on the condition to be treated, cross-reactivities of ingredients and pharmaco-properties of the combination. For example, when treating cancer, the compositions can be combined with other anti-cancer compounds (such as paclitaxel or rapamycin).

It is also possible to combine a compound of the disclosure with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-cancer effect denotes an anti-cancer effect that is greater than the predicted purely additive effects of the individual compounds of the combination.

Combination therapy is further described by U.S. Pat. No. 6,833,373 (McKearn et al.), which includes additional active agents that can be combined with the compounds described herein, and additional types of cancer and other conditions that can be treated with a compound described herein.

Accordingly, it is an aspect of this disclosure that a DNQ_{d} or DNQ can be used in combination with another agent or therapy method, preferably another cancer treatment. A DNQ_{d} or DNQ may precede or follow the other agent treatment by intervals ranging from minutes to weeks. In embodiments where the other agent and expression construct are applied separately to the cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with the active agent(s). In other aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the active agent(s). In certain other embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the active agent(s). In some situations, it may be desirable to extend the time period for treatment significantly, however, where several weeks (e.g., about 1, about 2, about 3, about 4, about 6, or about 8 weeks or more) lapse between the respective administrations.

Administration of the chemotherapeutic compositions of the present disclosure to a patient will follow general protocols for the administration of chemotherapeutics, taking into account the toxicity, if any. It is expected that the treatment cycles would be repeated as necessary. It also is contemplated that various standard therapies or adjunct cancer therapies, as well as surgical intervention, may be applied in combination with the described active agent(s). These therapies include but are not limited to chemotherapy, radiotherapy, immunotherapy, gene therapy and surgery.

i. Chemotherapy

Cancer therapies can also include a variety of combination therapies with both chemical and radiation based treatments. Combination chemotherapies include the use of chemotherapeutic agents such as, cisplatin, etoposide, irinotecan, camptostar, topotecan, paclitaxel, docetaxel, epothilones, taxotere, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib hydrochloride), antibodies to EGFR, GLEEVEC™ (imatinib), intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatine, vinblastine, vincristine, vindesine, bleomycin, doxorubicin, dactinomycin, daunorubicin, epirubicin, idarubicin, mithramycin, deoxycoformycin, Mitomycin-C, L-Asparaginase, teniposide, 17α-Ethinylestradiol, Diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin, herceptin, Bexxar, Velcade, Zevalin, Trisenox, Xeloda, Vinorelbine, Porfimer, Erbitux™ (cetuximab), Liposomal, Thiotepa, Altretamine, Melphalan, Trastuzumab, Lerozole, Fulvestrant, Exemestane, Fulvestrant, Ifosfomide, Rituximab, C225, Campath, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, paclitaxel, gemcitabine, navelbine, farnesyl-protein transferase inhibitors, transplatinum, 5-fluorouracil, vincristine, vinblastine and methotrexate, or any analog or derivative variant of the foregoing.

ii. Radiotherapy

Other factors that cause DNA damage and have been used extensively include what are commonly known as gamma rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (e.g., 3 to 4 wks), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

iii. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

iv. Gene Therapy

In yet another embodiment, the secondary treatment is a secondary gene therapy in which a therapeutic polynucleotide is administered before, after, or at the same time a first chemotherapeutic agent. Delivery of the chemotherapeutic agent in conjunction with a vector encoding a gene product will have a combined anti-hyperproliferative effect on target tissues.

v. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

### IV. EXAMPLES

The following Examples are intended to illustrate the above disclosure and should not be construed as to narrow its scope. One skilled in the art will readily recognize that the Examples suggest many other ways in which the disclosure could be practiced. It should be understood that numerous variations and modifications may be made while remaining within the scope of the disclosure. The disclosure may be further understood by the following non-limiting examples.

### Example 1— Materials and Methods

Mice. Female C57BL/6J and Rag1^{-/-} mice were purchased from UT southwestern mice breeding core. Myd88^{-/-}, Tlr4^{-/-}, Tlr9^{-/-}, Batf3^{-/-} and OT1CD8⁺ T cell receptor (TCR)-Tg mice in the C57BL/6J background and NSG-SMG3 mice were purchased from The Jackson Laboratory. Ifnar1^{-/-} mice were provided by Dr. Anita Chong from the University of Chicago. All the mice were maintained under specific pathogen-free conditions. Animal care and experiments were carried out under institutional and National Institutes of Health protocol and guidelines. This study has been approved by the Institutional Animal Care and Use Committee of the University of Texas Southwestern Medical Center.

Cell lines and Reagents. MC38, TC-1, B16, Panc02, Ag104Ld and A549 cells were cultured in DMEM or RPMI 1640 medium supplemented with 10% heat-inactivated fetal bovine serum, 100 U/ml penicillin and 100 U/ml streptomycin under 5% CO₂ at 37° C. β-Lapachone was synthesized as described (Pink et al., 2000) and dissolved in DMSO for in vitro study. Catalase, dicoumarol and FTY720 were purchased from Sigma-Aldrich. OT-1 peptide and OVA protein were from ThermoFisher. Anti-CD4 (GK1.5), anti-CSF1R (AFS98), anti-IFNAR1 (MAR1-5A3), anti-PD-L1 (10F.9G2), anti-CD8 (YTS) and anti-HMGB1 mAbs were purchased from BioXCell.

Sulforhodamine B (SRB) Cytotoxicity Assay. 4,000 cells were planted in 96 or 48 well plate with triplicates. After overnight growth, cells were exposed to β-lapachone with or without NQO1 inhibitor dicoumarol (50 μM) for a 3-hour pulse. After that, the cell supernatant was replaced by fresh medium, and the plate was incubated at 37° C. in a humidified incubator with 5% CO₂ for another 2 or 4 days. Following treatment, the culture supernatant was removed, and fixative reagents were gently added to each well. The wells were washed with water and the plate was air dried overnight. 1004, SRB Dye solution was added and incubated for 30 min, followed by washing and air dry. The cell growth was determined by the absorbance at 560 nM in a microplate reader (SpectroSTAR Nano, BMG Labtech). % Cell growth=(100×(Cell Control−Experimental))÷(Cell Control).

NQO1 knockout and overexpression. NQO1 gene in MC38 cells was knocked out by CRISPR/Cas9 technology. The guide sequence 5′-TTGTGTTCGGCCACAATATC-3′ was cloned into pSpCas9 (BB)-2A-Puro plasmid (Addgene, #62988) containing a puromysin selection gene. The plasmid was transiently transfected into MC38 cells. 48 h after transfection, puromysin resistant cells were selected and subcloned under the selective culture medium. MC38 Cell clones (#1, #2, and #5) without NQO1 expression were used for in vitro and in vivo studies. For NQO1 overexpression, B16 cells (NQO1 null) was transiently transfected with a full-length mouse NQO1 protein expression vector (pCMV3-HA-NQO1). The NQO1 stable expressing cells were selected and subcloned under the hygromycin containing culture medium. B16 cells clones (#1, #3 and #4) with NQO1 stable expression were used for following studies. The NQO1 expression levels were determined by western blotting assay.

Tumor Growth and Treatment. Approximately 6×10⁵ MC38 cells or 1.5×10⁵ TC-1, or 1.5×10⁵ B16 cells were subcutaneously inoculated into the right flank of mice. Tumor bearing mice were randomly grouped into treatment groups when tumors grew to certain sizes. For β-lap monotherapy, tumor bearing mice were treated with β-lap locally (intratumorally, 0.03 mg, 0.1 mg or 0.3 mg every other day for four times) or systemically (intravenously or intraperitoneally, 25 or 30 mg/kg every other day for four or six times). For CD4 and CD8 T cell depletion, 200 μg of antibodies were intraperitoneally injected four times at three days interval. For Macrophage depletion, 100 μg of Anti-CSF1R mAb were intratumorally injected three times at three days interval during β-lap treatment. For type I IFN blockade experiment, 150 μg of anti-IFNAR1 blocking mAbs were intratumorally injected at three days interval for a total three times. The blocking and depletion experiments above started one day before the first β-lap treatment. For HMGB1 blockade experiments, 200 μg of anti-HMGB1 mAbs were administered intraperitoneally (i.p.) every three days for total three times staring at the same day of the first β-lap treatment. For PD-L1 checkpoint blockade combination therapy, 100 μg (for the MC38 model) or 150 μg anti-PD-L1 (clone 10F.9G2) was administered intraperitoneally to tumor bearing mice every three days for total three times starting at the same day of the first β-lap treatment. Tumor volumes were measured at least twice weekly and calculated as 0.5×length×width×height.

Immune reconstituted mouse models. For C57BL/6 Rag1−/− immune-reconstituted model (Lee et al., 2009; Tang et al., 2016), 2×10⁶ A549 cells were s.c. inoculated into female Rag1−/− mice. After the tumor was well established (about 100 mm³), 2×10⁶ total LN cells from OTI transgenic mice were intravenously injected into the tumor bearing mice one day before treatment. Later, the mice were treated with β-lap locally (i.t., 0.2 mg) every other day for four times. Tumor volumes were measured at least twice weekly.

For NSG-SGM3 humanized mouse model, four-week-old NSG-SGM3 female mice were irradiated with 100 cGy (X-ray irradiation with X-RAD 320 irradiator) one day prior to human CD34+ cells transfer. Irradiated mice were treated with Bactrim (Aurora Pharmaceutical LLC) water for 2 weeks. Cord blood was obtained from UT Southwestern Parkland Hospital. Human CD34+ cells were purified from cord blood by density gradient centrifugation (Ficoll® Paque Plus, GE healthcare) followed by positive immunomagnetic selection with anti-human CD34 microbeads (Stem Cell). 10⁵ CD34⁺ cells were intravenously injected into each recipient mouse. 12 weeks after engraftment, humanized mice with over 40% human CD45+ cells reconstitution and age and sex matched non-humanized mice were inoculated with 2×10⁶ A549 tumor cells subcutaneously on the right flank. At day 19, the tumor bearing mice were treated with β-lap locally (i.t., 0.2 mg) every other day for four times. Tumor volumes were measured at least twice weekly. All experiments were performed in compliance with UTSW Human Investigation Committee protocol and UTSW Institutional Animal Care and Use Committee.

HMGB1 Release Detection. Tumor cells were planted in 6-well plate and grown to 70% confluence and treated with increasing concentration of β-lap for 3 hours, followed by washing and medium replacement. The supernatant was assayed for extracellular HMGB1 24 h later using an ELISA KIT (Chondrex).

IFNγ Enzyme-Linked Immunosorbent Spot Assay (ELISPOT). Tumor drain LNs and spleen from tumor bearing mice were collected and single cell suspension was prepared. Irradiated tumor cells or OT-1 peptides were used to re-stimulate the tumor specific T cells. In general, a total of 2-4×10⁵ LN cells or splenocytes and 2-4×10⁵ irradiated tumor cells were cocultured for 48 hours, and ELISPOT assay was performed using the IFNγ ELISPOT kit (BD Bioscience) according to the manufacturer's instructions. Spots were calculated by ImmunoSpot Analyzer (Cellular Technology Limited).

Cell Isolation from Tissues. CD11c⁺ DCs or CD8⁺ T cells were isolated from lymph nodes or spleen of mice with a positive CD11c isolation kit or a negative CD8 isolation kit (Stemcell) according to the manufacturer's instructions. For tumor single cell suspension, tumor tissues were cut into small pieces, and resuspended in digestive buffer (1.5 mg/ml type I collagenase and 100 μg/ml DNase I) for 45 minutes in a 37° C. shaking incubator. After digestion, cells were passed through a 70-μm cell strainer.

Flow Cytometric Analysis. Tumor cell suspension was blocked with the anti-CD16/32 antibody (clone 2.4G2) for 10 min, and then incubated with indicated antibody for 30 min at 4° C. in the dark. Fixable viability Dye eFlour 506 (eBioscience) was used to exclude the dead cells. Sample was analyzed on cytoFLEX (Backman coulter) flow cytometer.

DCFDA Cellular ROS Detection Assay. The level of cellular ROS was determined by the DCFDA-Cellular ROS Detection Assay Kit (Abcam) according to the manufacturer's instructions. Briefly, cells were plated into 12-well plates and grown to about 70% confluence, and stained with DCFDA at 37° C. for 30 min. After that cells were treated with different concentration of β-lap for 3 hours. ROS signal was determined using Flow cytometry at Ex/Em: 485/535 nm.

Statistical Analysis. All the data analyses were performed with GraphPad Prism statistical software and shown as mean±SEM. *P<0.05, **P<0.01, ***P<0.001 and ****P<0.0001 determined by two-way ANOVA or unpaired two-tailed t tests. A value of p<0.05 was considered statistically significant.

### Example 2—Results

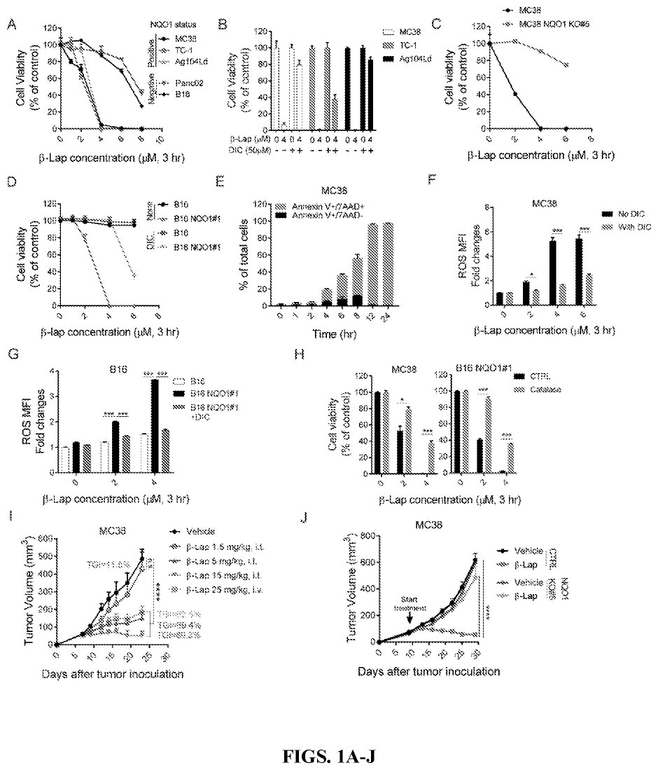

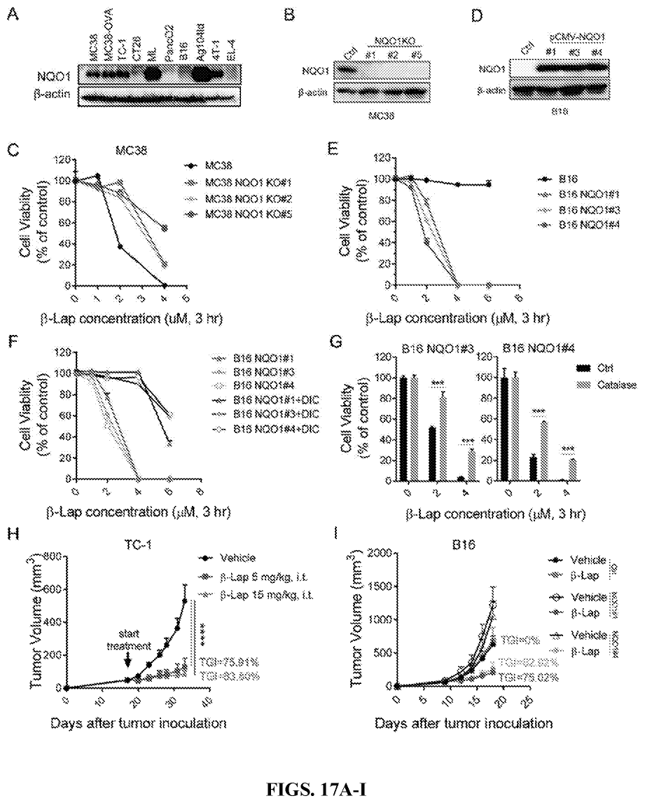

β-lap suppresses murine tumor growth in a NQO1 dependent manner both in vitro and in vivo. Multiple murine cancer cell lines were used to examine the role of NQO1 in β-lap function. Tumor cell lines (MC38 colorectal adenocarcinoma, TC-1 lung cancer and Ag104Ld fibrosarcoma) that express high level of NQO1 (FIG. 17A) were sensitive to 3 h β-lap exposure (FIG. 1A). In contrast, NQO1 deficient cell lines, B16 (melanoma) and Panc02 (pancreatic cancer) (FIG. 17A), were resistant to β-lap exposure (FIG. 1A). Dicoumarol, an NQO1 specific inhibitor, reversed the NQO1-mediated lethality (FIG. 1B). Next, the inventors determined whether depletion of NQO1 abrogates the cytotoxicity of β-lap. CRISPR-mediated NQO1 knockout (FIG. 17B) endowed MC38 cells resistance to β-lap treatment (FIG. 1C; FIG. 17C). Similarly, overexpression of NQO1 in B16 cells (FIG. 17D) led to sensitivity to β-lap (FIG. 1D; FIG. 1E), and inhibition of NQO1 by dicoumarol spared β-lap lethality (FIG. 17F). Lethal dose of β-lap caused rapid cell swelling, membrane rupture and Annexin V+/7AAD+ cell death without caspase activation (FIG. 1E and data not shown). NQO1 catalyzes the two-electron oxidoreduction of β-lap to generate high levels of ROS (i.e., hydrogen peroxide/H₂O₂), causing massive DNA oxidation and cell death (Huang et al., 2016). Indeed, β-lap-induced high level of ROS in NQO1 positive murine tumor line and much less in NQO1 null lines (FIGS. 1F-1G). Inhibition of NQO1 by dicoumarol abolished this effect (FIGS. 1F-G). Next, the inventors determined whether neutralizing ROS could inhibit β-lap induced cell lethality. Catalase, an H₂O₂ scavenging enzyme, significantly spared β-lap-mediated lethality (FIG. 1H; FIG. 17G). These results suggest that β-lap induces NQO1+ tumor cell death through intensive tumor specific ROS production in vitro.

The inventors further examined the antitumor efficacy of β-lap in three subcutaneous syngeneic tumor models: MC38, TC-1 and B16 each with different NQO1 levels. In the MC38 tumor model, 25 mg/kg of β-lap was systemically (intravenously) administered to tumor bearing WT C57BL/6 mice on day 7 after tumor inoculation. Treatment with β-lap resulted in marked tumor inhibition (FIG. 1I). β-Lap might act on various cells when delivered systemically. To mechanistically explore whether and how β-lap functions within the local tumor environment for tumor regression, various doses of β-lap were intratumorally injected every other day for total 4 doses. Local treatment also significantly suppressed tumor growth in a dose dependent manner. Tumor growth inhibition rates (TGI, %) were 62.5% for mice with systemic β-lap treatment and 11.8%, 69.4% and 89.3% for mice with local β-lap (1.5 mg/kg mg, 5 mg/kg and 15 mg/kg mg) treatments (FIG. 1I). Notably, local treatment with much lower dose of β-lap (15 mg/kg) induced more robust tumor regression than systemic treatment and 46.7% (7/15) of mice achieved complete tumor rejection (FIG. 1I), suggesting that major action site might be related to local tumor microenvironment. Consistent with the in vitro study, β-lap's therapeutic effect was abolished in NQO1 knockout MC38 mouse models. (FIG. 1J). Similarly, in TC-1, HPV E6/E7 transformed tumor model, β-lap treatment also led to significant tumor suppression (FIG. 17H). To further confirm the specificity of β-lap in vivo, the inventors established subcutaneous xenografts using parental NQO1 deficient or NQO1 overexpressing B16 cells clone (#1 and #4) in WT C57BL/6 WT mice. As expected, NQO1null tumors didn't response to β-lap treatment (FIG. 17I). In sharp contrast, NQO1 overexpressing (clone #1 and #4) tumor bearing mice showed a dramatic tumor suppression after β-lap treatment (FIG. 17I). Of note, B16 tumors overexpressing NQO1 grew much faster than B16 parent cells, indicating that NQO1 promotes in vivo tumor growth (Oh et al., 2016). These results provide evidence that β-lap selectively suppresses NQO1 positive murine tumor growth in vitro and in vivo. NQO1 is essential and sufficient for β-lap mediated antitumor effect.

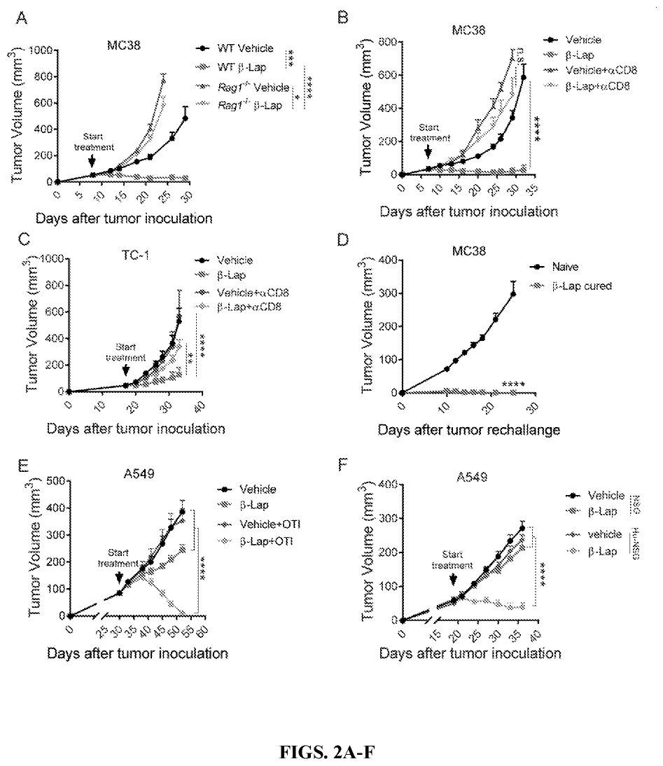

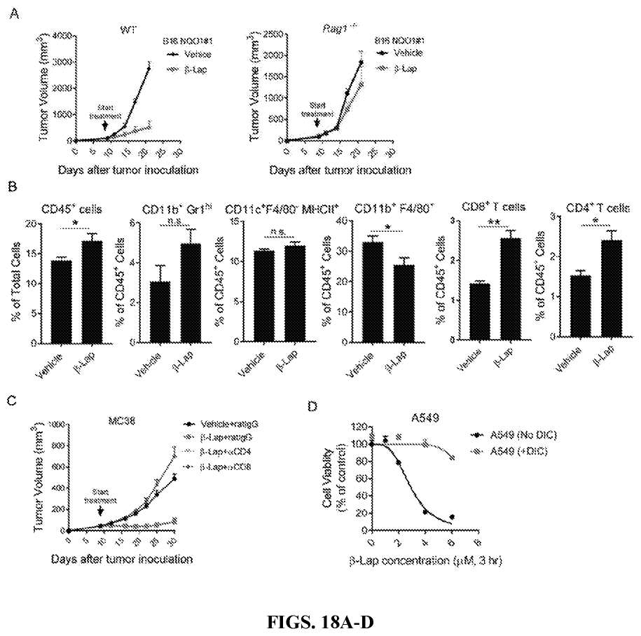

β-Lap mediated-antitumor effect depends on CD8⁺ T cells. Most studies on how β-lap kills tumor cells have focused on cancer cell autonomous mechanism (Huang et al., 2016; Pink et al., 2000; Li et al., 2016). Here, the inventors asked whether β-lap-mediated antitumor effect involves the immune system. They established MC38 tumors in immunocompetent and immunodeficient mice respectively to study the effect of β-lap on adaptive immunity. After only 4 doses of β-lap treatment, MC38 tumor was eradicated in WT C57BL/6 mice with 50% of mice cured (FIG. 2A). Unexpectedly, β-lap lost therapeutic activity in immune-deficient Rag1 KO (Rag1^{-/-}) C57BL/6 mice (FIG. 2A). A similar effect was observed in NQO1 overexpressing B16 tumor model (FIG. 18A) and TC-1 model (data not shown), suggesting that the adaptive immune system is required for the profound antitumor effect of β-lap in vivo. Indeed, the inventors found an increase in CD4⁺ and CD8⁺ T cells in β-lap treated tumors compared to control MC38 tumors (FIG. 18B), suggesting that some subsets of T cells might contribute to adaptive immunity. To test which T cell subset was essential, mice were treated with either anti-CD4 or anti-CD8 depletion antibodies in conjunction with β-lap treatment. While treatment with β-lap alone or β-lap combined with CD4⁺ T cell depletion controlled MC38 tumor growth, CD8⁺ T cells depletion abolished β-lap's antitumor effect (FIG. 2B; FIG. 18C). This data suggests that CD8⁺ T cells, but not CD4⁺ T cells, are required for β-lap-mediated tumor regression. Similar results were obtained in mice bearing TC-1 tumors (FIG. 2C). To determine whether β-lap-mediated antitumor responses result in prolonged protective T cell immunity, the inventors re-challenged the mice that underwent complete MC38 tumor rejection after β-lap treatment with a lethal dose of MC38 cells. All the β-lap cured mice rejected the re-challenged tumors (FIG. 2D), indicating the generation of memory T cells after β-lap treatment.

To test the efficacy of β-lap in controlling human tumors, the inventors developed two xenograft models in immune-reconstituted C57BL/6 Rag1^{-/-} (FIG. 2E) and NSG-SGM3 mice (FIG. 2F). Human lung carcinoma cell line A549 that highly expresses NQO1 was sensitive to β-lap treatment in vitro (FIG. 18D). In C57BL/6 Rag1^{-/-} immune-reconstituted model (Lee et al., 2009; Tang et al., 2016), A549 cells were s.c. inoculated. After the tumor was well established (about 100 mm³), a total of 2×10⁶ lymph node (LN) cells from OTI transgenic mice were transferred into tumor-bearing Rag1^{-/-} mice to reconstitute a small number of T cells without reducing tumor growth. LN cells from OTI transgenic mice contain about 98% OVA-specific T cell that cannot respond to human tumor antigens but suppress homeostatic proliferation of a small number of non-OTI T cells. A small fraction of non-OT-1 T cells have the potential to recognize human tumor antigens, which approximates 200-1000 clones, the number of tumor-reactive T cell in human patients. Without T cell transfer, β-lap treatment only partially inhibited A549 tumor growth. However, in the present of T cells, β-lap induced more robust tumor regression with 50% of tumors completely rejected (FIG. 2E), indicating that β-lap-mediated antitumor effects are largely dependent on T cells. To study the T cell function in β-lap mediated tumor suppression in a human-specific system, the inventors further developed a next-generation humanized xenograft model with NSG-SGM3 mice that support human myeloid cell proliferation and provide in vivo conditions that better mimic the human immune system and natural tumor microenvironment (Morton et al., 2016). NSG-SGM3 mice injected with human CD34+ hematopoietic stem cells (Hu-NSG-SGM3) showed robust engraftment efficiencies measured by flow cytometry using human CD45, CD3, CD4 and CD8 leukocyte markers. Naïve NSG-SGM3 and Hu-NSG-SGM3 mice were separately inoculated with A549 cells and were treated with β-lap. Notably, in the presence of the human immune system, β-lap treatment induced much better tumor control, indicating that the reconstituted immune system restored the antitumor effect of β-lap (FIG. 2F). The inventors further investigated tumor infiltrated immune cells in the tumor microenvironment, and found a significant increase of CD45+ cells, and CD8⁺ T cells but not CD4⁺ T cells in the tumor tissues after β-lap treatment. Together, these data reveal the necessity of T cells in β-lap induced tumor control.

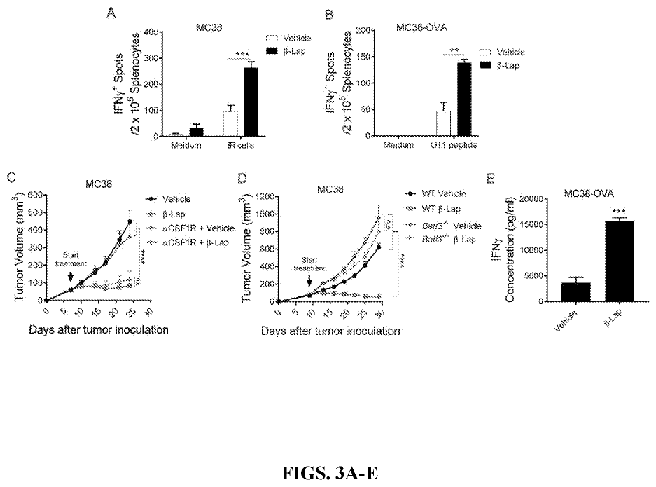

I3-Lap-induced-antitumor effect depends on dendritic cell-mediated T cell cross-priming. Given that CD8⁺ T cells are essential for the anti-tumor efficacy of β-lap, the inventors hypothesized that β-lap treatment increases the antigen specific T cell response. To rule out of direct effect of β-lap on CD8 T cells, the inventors evaluated the expression of NQO1 on CD8 T cells and the effect of β-lap on CD8 T cell survival, proliferation and function. The results showed that neither naïve CD8 T from spleen nor tumor infiltrating CD8 T cell expressed NQO1. Moreover, tumor lethal dose of β-lap had no effect on CD8 T cell apoptosis and on anti-CD3/CD28stimulated cell proliferation and IFNγ production. To test if β-lap increases the antigen specific T cell response, splenocytes from MC38 tumor-bearing mice were collected 10 days after the initial β-lap treatment and an IFGNy ELISPOT assay was performed to measure the effector function of activated T cells. As shown, in the presence of tumor stimulation (irradiated tumor cells), IFNγ-producing T cells dramatically increased in the β-lap treatment group (FIG. 3A). The inventors further generated an MC38-OVA cell line using OTI peptide to better track T cell responses. Similarly, in the IFNγ ELISPOT assay OTI peptide, the numbers of OTI specific T cells were much higher in spleens of mice after β-lap treatment (FIG. 3B). The increase of tumor-specific CD8 cells suggests that β-lap treatment might induce cross-priming and reactivate T cells to control the tumor growth. Cross-presentation by APCs such as dendritic cells (DCs) or macrophages is considered the major priming mechanism to activate tumor specific T cells. To further nail down which APCs are essential β-lap induced antitumor effect, the inventors first used anti-CSF1R Ab to deplete macrophages in the tumor tissue (Tang et al., 2018). As shown, macrophage depletion did not affect the response of MC38 bearing mice to β-lap treatment (FIG. 3C). Batf3-dependent DCs CD8α⁺ or CD103⁺ DCs) specialize in cross-presentation of necrotic tumor cell-derived epitopes to directly activate CD8⁺ T cells (Sanchez-Paulete et al., 2016; Broz et al., 2014; Salmon et al., 2016). Batf3^{-/-} mice lack functional CD8α⁺ or CD103⁺ DCs and have impaired cell cross-presentation activity. In WT mice, β-lap treatment induced a robust tumor regression (FIG. 3D). In stark contrast, no therapeutic effect was seen in identically treated Batf3−/− mice (FIG. 3D). To further address the possibility that β-lap could increase cross-presentation, the inventors used an antigen-specific system to track the priming and activation of tumor antigen specific T cells. WT mice bearing MC38-OVA tumors were treated with β-lap, then DCs were isolated from the tumor-drain lymph nodes (TdLN) and co-cultured with CD8 T cells from OTI transgenic mice. IFNγ secretion was measured to evaluate the capability of DCs to prime the antigen specific T cells. Indeed, after β-lap treatment, DCs induced more IFNγ production by OTI T cells (FIG. 3E). These results suggest that DC-mediated cross-priming is required for the β-lap-induced tumor regression and tumor reactive T cell response.

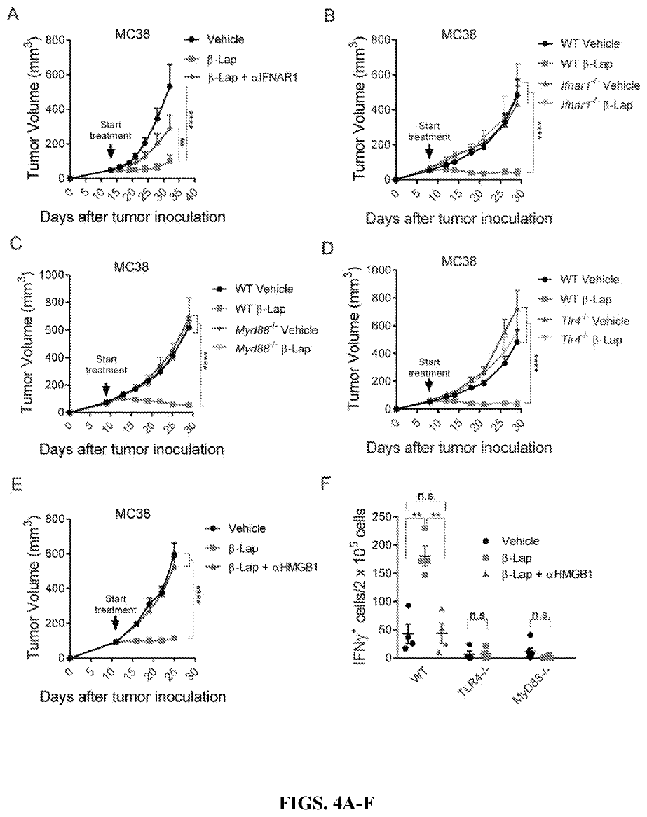

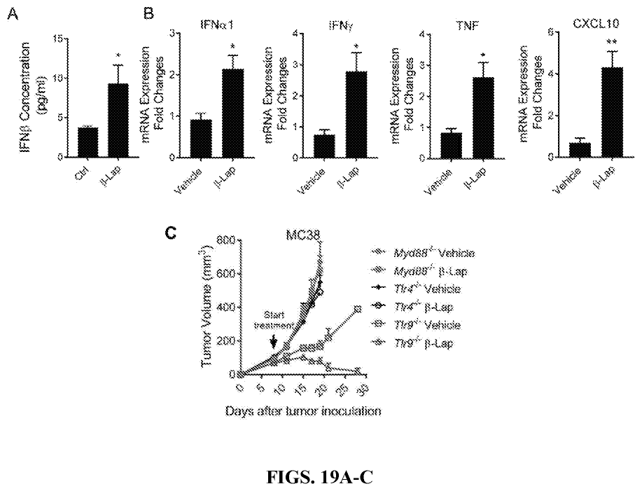

Type I IFNs and TLR4/MyD88 signaling is required for the antitumor effect of β-lap and tumor specific CTLs. APCs in the tumor microenvironment are dysfunctional, leading to ineffective priming and activation of T cells (Lee et al., 2009; Corrales et al., 2017). Type I interferon (IFN) is essential for optimal cross-priming of T cells (Corrales et al., 2017; Deng et al., 2014). The inventors further examined whether type I IFN signaling was required for the therapeutic effect of β-lap. IFNAR1 blocking antibody was intratumorally administrated to neutralize type I IFN signaling in the tumor microenvironment. Strikingly, blocking type I IFN significantly diminished the therapeutic effect of β-lap (FIG. 4A). To determine whether tumor cell or host IFN signaling was essential, MC38 tumors was inoculated into WT and IFNAR1 deficient (Ifnar−/−) C57BL/6 mice, followed by β-lap treatment. The results showed that the therapeutic effect was abrogated in mice with impaired IFNAR1 signaling (FIG. 4B). As expected, the inventors observed upregulated IFNα/β and IFNs response genes CXCL10 as other cytokines such as IFNγ and TNFα in tumors treated β-lap (FIG. 19A-B). It has been demonstrated that MyD88 is involved in type I IFN production and antitumor immunity by some chemotherapy agents (Sistigu et al., 2014; Zitvogel et al., 2015; Apetoh et al., 2007). To determine whether MyD88 signaling is required for β-lap treatment, tumors cells were s.c. implanted into WT and MyD88 deficient (Myd88−/−) mice. β-lap induced tumor regression disappeared in Myd88−/− mice with the same therapeutic schema (FIG. 4C), demonstrating the host MyD88 was indispensable for the antitumor effect of β-lap. Because β-lap can induce robust tumor cell death, the inventors hypothesized that this in turn resulted in the secretion of damage-associated molecular patterns (DAMPs) and exposure of tumor antigens, thereby boosting antitumor immune responses. Interestingly, knockout of TLR4 but not TLR9, both of which are the major upstream receptors of MyD88 signaling to sense the DAMPs, also led to the similar therapeutic resistance (FIG. 4D; FIG. 19C). Previous studies have shown that HMGB1 (and HMGB1/DNA complexes), one of the endogenous ligands of TLR4, can function as a danger signal that stimulates DC cross-priming in a MyD88-dependent fashion (Apetoh et al., 2007). To determine whether β-lap-induced tumor regression is HMGB1-dependent, anti-HMGB1 mAb was administered to neutralize free HMGB1 along with β-lap treatment. The results showed that blockade of HMGB1 signaling diminished the effect of β-lap (FIG. 4E), indicating that β-lap may induce HMGB1 release in the tumor microenvironment to enhance innate response via a TLR4/MyD88 pathway. To further address the essential role of HMGB1, the inventors evaluated the tumor specific T cell response when blocking signaling in conjunction with β-lap treatment. MC38 tumor cells were s.c. implanted into WT or Tlr4−/− or Myd88−/− C57BL/6 mice, and tumor bearing mice were treated with β-lap with or without anti-HMGB1 Ab. After treatment, lymphocytes from tumor-draining lymph nodes (TdLN) were collected and subjected to IFNγ ELISPOT assays. In WT mice, β-lap treatment increased the number of tumor-reactive T cells and this effect was abolished when co-administrated with anti-HMGB1 neutralizing Ab (FIG. 4F). Similarly, in Tlr4−/− and Myd88−/− mice there were much less tumor reactive T cells in the control group compared with that in WT mice (FIG. 4F). More importantly, β-lap treatment could not enhance the tumor specific T cell response in these deficient mice (FIG. 4F). These results suggest that the TLR4/MyD88/type I IFNs signaling cascade is required for β-lap induced innate and adaptive antitumor immune response.

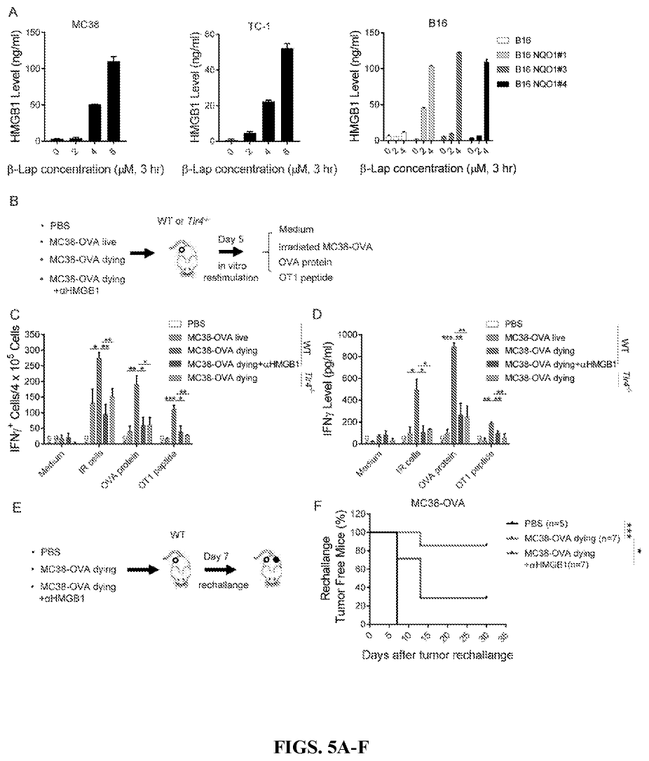

β-Lap treatment induces tumor immunogenic cell death and triggers HMGB1-dependent antitumor T cell immunity in vivo. The HMGB1-dependent tumor specific T cell response and HMGB1-dependent antitumor effect of β-lap in vivo suggested that β-lap could potentially induce immunogenic cell death (ICD) in the tumor. To test this hypothesis, the inventors checked the ICD hallmark: HMGB1 secretion in β-lap treated tumor cells in vitro. Indeed, they observed a dose-dependent secretion of HMGB1 in NQO1+ tumor cells (MC38, TC-1 and NQO1-overexpressing B16 cells) but not in NQO1− cells (B16 parent cells) (FIG. 5A). Inhibition of NQO1 by dicoumarol abolished β-lap-induced HMGB1 secretion (data not shown). The inventors presumed that HMGB1 exposure might dictate the immunogenicity of β-lap-induced tumor cell death. HMGB1 was found to be critical for β-lap-induced immunogenicity in two experiments: (i) Dying MC38-OVA cells induced by β-lap in vitro were injected into the flank of C57BL/6 mice in conjunction with or without anti-HMGB1Ab (FIG. 5B). The numbers of tumor antigen-specific T cells (FIG. 5C) and IFNγ production (FIG. 5D) in TdLN were determined; (ii) C57BL/6 mice that received vaccination with β-lap-induced dying MC38-OVA cells in the presence of anti-HMGB1Ab were rechallanged with MC38-OVA for evaluation of the antitumor protection (FIGS. 5E-F). As shown, mice vaccinated with β-lap-induced dying cells had more IFNγ-producing antigen specific T cells compared to mice vaccinated with living cells (FIGS. 5C-D). However, the tumor-specific T cell responses diminished when vaccinated with dying cell and anti-HMGB1Ab mixture (FIGS. 5C-D). Consistently, TLR4−/− mice also showed reduced tumor reactive T cells and IFNγ production compared to WT mice when vaccinated with identical dying cells (FIGS. 5C-D). To test the ability of β-lap-induced dying cells to activate the adaptive immune system, the inventors used a prophylactic tumor vaccination model in immunocompetent C57BL/6 mice (FIG. 5E). Immunization of mice with β-lap-induced dying cells prevented the growth of the re-challenged tumor (FIG. 5F). Notably, the antitumor protection effect decreased when mice were vaccinated with dying cells and anti-HMGB1 neutralized antibody (FIG. 5F). These results indicate that β-lap induces ICD and enhances antitumor immunogenicity in an HMGB1 dependent manner.

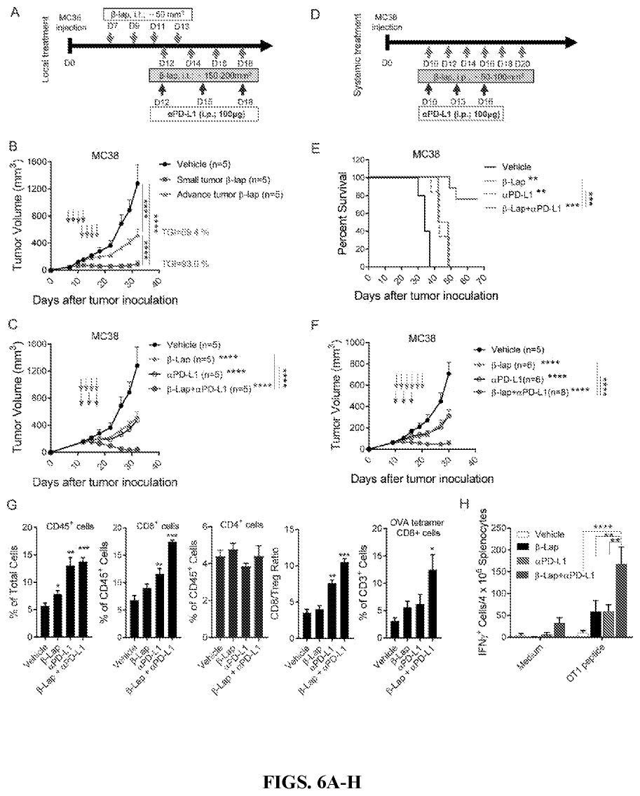

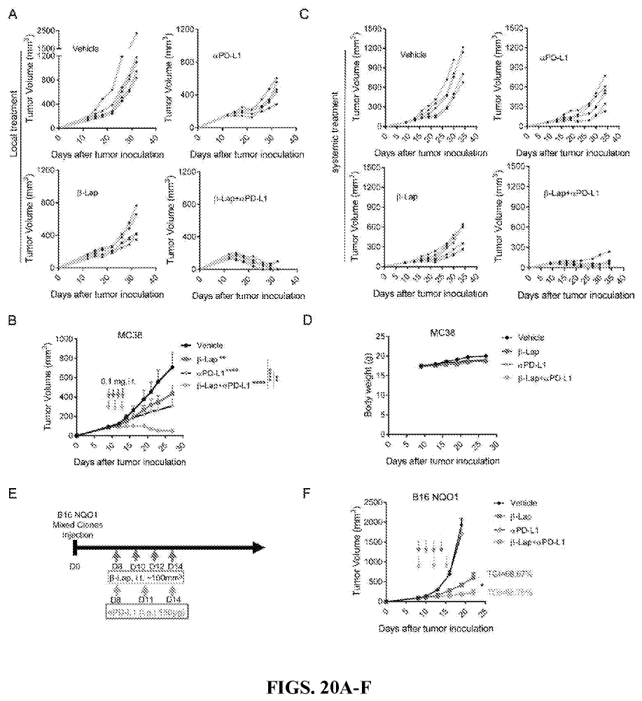

β-Lap eradicates large established and checkpoint blockade refractory tumors by combination with anti-PD-L1 therapy. In clinical practice, patients with well-established tumors may generate complicated immunosuppressive networks and are generally refractory to immunotherapy (Sharma et al., 2017; Smyth et al., 2016). Similarly, in our preclinical model, complete tumor rejection was achieved only in mice bearing small tumors (about 50 mm³) after β-lap treatment (TGI, 93.0%; FIGS. 6A-B), and large established tumors (about 150-200 mm³) were only partially controlled by identical treatment protocols (TGI, 59.36%; FIGS. 6A-B). The finding that β-lap provokes an innate and adaptive immune response as part of its mechanism of action paved a path for for the combination of β-lap with T cell checkpoint blockade (FIG. 6A) to eradicate the advanced and checkpoint blockade refractory tumors. To test this, the inventors next combined local β-lap treatment (15 mg/kg, i.t.) with anti-PD-L1 treatment in mice bearing established large MC38 tumors. The advanced MC38 tumors only showed a moderate response to anti-PD-L1 alone (TGI, 63.25%, FIG. 6C). In stark contrast, mice in the combination groups showed robust tumor control and regression (TGI, 96.86%; FIG. 6C; FIG. 20A). Notably, 60% of tumor-bearing mice completely rejected their tumors on combination treatment (FIG. 6C; FIG. 20A). Interestingly, synergistic effects of β-lap and immunotherapy were also observed using a much lower dose (5 mg/kg, i.t.) of β-lap locally (FIG. 20B). Recent studies showed that cancer immunotherapy is enhanced by local and abrogated by systemic chemotherapy treatment (Mathios et al., 2016; Ariyan et al., 2018). To further evaluate if systemic β-lap treatment has any immunosuppressive effects and impairs anti-PD-L1 immunotherapy, MC38 tumor bearing mice were administered systemic β-lap treatment (30 mg/kg, i.p.) monotherapy or combined with anti-PD-L1 (FIG. 6D). Monotherapy of β-lap or anti-PD-L1 led to similarly moderate inhibition of tumor growth (FIG. 6E; FIG. 20C). Combination treatment had a synergistic effect on their antitumor action with 25% of tumors completely rejected (FIG. 6E; FIG. 20C) and markedly improved the survival of tumor bearing mice (FIG. 6F). B16 tumors express PD-L1 but have poor immunogenicity and are not responsive to PD-L1/PD-1 immune checkpoint blockade (Chen et al., 2015; Curran et al., 2010). The inventors used their NQO1 overexpressing B16 tumor model to evaluate the therapeutic efficacy of β-lap and anti-PD-L1 combination treatment (FIG. 20E). As expected, NQO1 overexpressing B16 tumors failed to respond to anti-PD-L1 Ab alone (FIG. 20F). By contrast, β-lap monotherapy largely inhibited the growth of the B16-NQO1 tumors (TGI, 68.67%). Strikingly, when combined with PD-L1 blockade, β-lap had a marked synergetic antitumor effect (TGI, 88.76%; FIG. 20F).

To further elucidate the mechanism of synergy between β-lap and PD-L1 blockade, the inventors investigated tumor infiltrated immune cells in the tumor microenvironment and tracked antigen specific T cells in tumor tissues and spleen 12 days after the initial treatment in MC38-OVA tumor model. They found a significant increase of CD45⁺ cells, CD8⁺ T cells, and CD8⁺ T cell:Treg cell ratio in the tumor tissues with either β-lap or anti-PD-L1 monotherapy (FIG. 6G). Importantly, these effects were dramatically magnified in the combination group (FIG. 6G). They further tracked CD8⁺ T cells specific for the model antigen OVA257-264 (OTI peptide) in the tumor tissues and spleen. As shown, neither β-lap or anti-PD-L1 monotherapy increased the OT1 antigen specific T cells in the tumor tissues (FIG. 6G) and spleen (FIG. 6H). Strikingly, combined therapy robustly expanded the tumor antigen specific T cells in both tumor tissue and spleen (FIGS. 6G-H). These results suggest that β-lap treatment enhanced the tumor immunogenicity and increased T cell infiltration and tumor specific T cell response when combined with the PD-L1 blockade. These data demonstrate a potent synergy between β-lap and PD-L1 blockade in controlling large established and checkpoint blockade refractory NQO1-positive tumors.

β-Lap induces tumor-specific ROS and DNA damage and selectively promotes the programmed necrosis of NQO1 positive cells. NQO1, an enzyme specifically and uniquely elevated in multiple human cancers, including NSCLC, pancreatic cancer, colon cancer, breast cancer, and head and neck cancer, can be exploited in a tumor-selective manner for therapy. β-lap has been shown significant antitumor effects, especially when combined with PARP1 inhibitors. However, it is still unclear whether β-lap-mediated tumor specific DNA damage and cell death have some interaction with the immune system; whether β-lap evokes an immunogenic cell death and results in tumor regression which is dependent on host immune system. To these ends, the inventors examined the antitumor activity of β-lap in vitro and in immunocompetent mice. As shown, β-lap selectively killed NQO1⁺ murine tumor cell lines (MC38, TC-1, and Ag104Ld), and this effect can be suppressed by dicoumarol (DIC, a fairly specific NQO1 inhibitor) and do not occur in NQO1⁻ cells (B16, pan02) (FIGS. 8A-B). High level of H₂O₂ was produced after a 3 h exposure to β-lap in NQO1⁺ cells but not NQO1⁻ cells (FIG. 8C), which suggest an ideal targeted effect of β-lap. β-lap triggered NQO1⁺ cancer cells death by a unique caspase-independent manner (FIG. 8D) and induced PARP1-mediated programmed necrosis (FIG. 8E).

The antitumor efficacy of β-lap in mice requires host adaptive immune system. The inventors separately established immunocompetent and immunodeficient mice bearing NQO1-positive murine lung cancer cells, TC-1. After 3 doses of β-lap treatment, tumor regression occurred in TC-1 tumor-bearing WT mice, but not in adaptive immune-deficient rag^{-/-} mice (FIG. 9A). This result suggested that adaptive immune system was required for the profound antitumor efficacy of β-lap in vivo. To test which T cell subset was essential for controlling the burden, mice were treated with either anti-CD4 or anti-CD8 depletion antibodies in conjunction with β-lap treatment. While treatment with β-lap alone or β-lap combined with anti-CD4 antibody resulted in the similar tumor growth suppression, combination therapy with β-lap+anti-CD8 antibody caused complete failure of tumor growth inhibition compared to β-lap alone (FIG. 9B). These data suggested that CD8⁺ T cells were required for β-lap-mediated tumor regression. Similar results were obtained in mice bearing MC-38 cells, a murine colon cancer model.

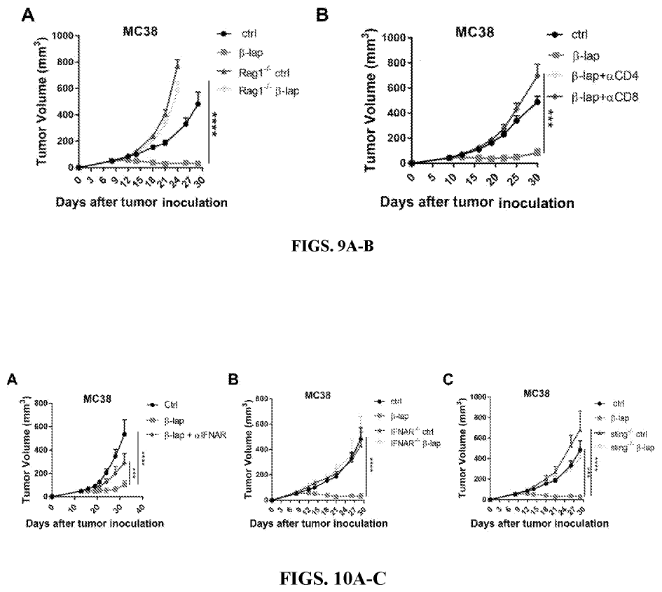

β-Lap induces tumor regression dependent on STING-dependent DNA sensing and type I IFNs signaling. Type I IFNs have emerged as potential key danger signals that initiate antitumor T cell responses after initiation of various antitumor therapies, bridging innate and adaptive immunity. To test whether type I IFNs are involved in the β-lap-induced tumor regression, the inventors generated two models to block type I IFNs signaling: injection of anti-IFNAR (interferon-alpha/beta receptor) blocking antibody in the tumor microenvironment (FIG. 10A) and knockout of host IFNAR gene in mice (FIG. 10). The inventors found that blockage of type I IFNs signaling in the microenvironment greatly impaired the antitumor efficacy of β-lap (FIG. 10A). Consistent with these results, β-lap-induced tumor regression was totally abrogated in IFNAR knockout mice, compared to that in WT mice (FIG. 10B), suggesting that type I IFNs might be the cytokines essential for β-lap-mediated tumor regression. Recently, both TLRs/MyD88 pathway and STING-mediated cytosolic DNA sensing cascade have been demonstrated to be major mechanisms of type I IFNs production. To investigate whether MyD88 and STING signaling pathways are required to mediate response to β-lap treatment, TC-1 cells were implanted in WT, MyD88 or STING deficient mice. The inventors found that, after β-lap treatment, while tumor burden was significantly reduced in WT mice (FIG. 10C) and MyD88 deficient mice (data not shown), absence of host STING significantly impaired the antitumor efficacy of β-lap (FIG. 10C). These results suggest that STING-dependent cytosolic DNA sensing and type I IFNs production are critical for the therapeutic effect of β-lap in vivo.

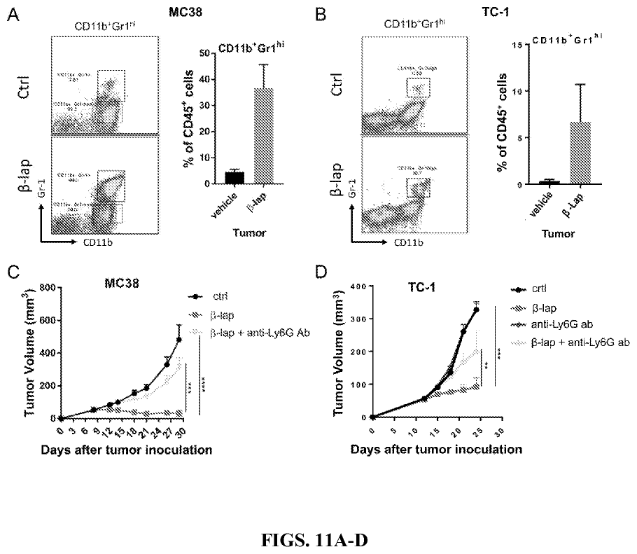

Tumor-infiltrating neutrophils are required for antitumor efficacy of β-lap in vivo. Type I IFNs is known to bridge innate and adaptive immune and be critical for the cross-priming of tumor-specific T cells response. The inventors proposed that treatment of β-lap can trigger innate sensing by increasing some danger signaling and recruiting some phagocytes and lead to cross-presentation of tumor antigen. To test this idea, they dissected the immune cells population in the tumor microenvironment after 3 days of β-lap treatment. Interestingly, they found tumor-infiltrating neutrophils (CD11b⁺Gr1⁺ subset) were significantly increased (FIG. 11A), while percentages of macrophage, DC, CD8⁺ and CD4⁺ T cells were less affected. To investigate whether this increased neutrophil subset was involved in the β-lap-induced tumor regression, the anti-Ly-6G (clone 1A8) antibody, which specifically targets tumor-infiltrating neutrophils, was used to deplete this subset. Antibody-mediated neutrophils depletion greatly impaired the therapeutic effect of β-lap in vivo (FIG. 11B), suggesting the critical role of newly neutrophil infiltration in the antitumor immune response.

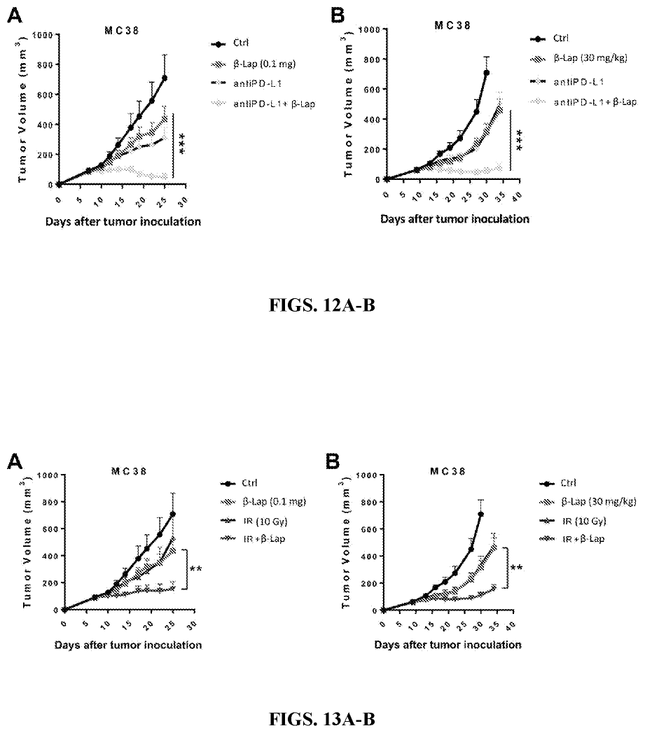

β-Lap can synergize with immune checkpoint blockade (anti-PD-L1/PD-1) therapy to efficaciously kill NQO1⁺ tumor cells. The finding that β-lap can provoke an innate immune response as part of its mechanism of action has profound implications for its combination with adaptive T-cell-based immune checkpoint blockade strategies, such as PD-L1/PD-1 inhibitors, to further activate adaptive immunity. Type I IFNs-induced upregulation of PD-L1 in the tumor microenvironment is one of the major reasons for acquired tumor resistance to multiple treatments, which leads to the therapeutic window for combination therapy between β-lap+PD-L1/PD-1 inhibitors. To test this hypothesis, the inventors then established subcutaneous xenografts using 6×10⁵ MC38 cells in C57BL/6J WT mice. Mice (n=5) were treated intratumorally (i.t.) (FIG. 12A) or intraperitoneally (i.p.) (FIG. 12B) with HPβCD vehicle alone, anti-PD-L1 (Atezolizumab) or 0.1 mg (i.t.) or 30 mg/kg (i.p.) of β-Lap with or without anti-PD-L1 every three days for 4 injections. Treatment was started when tumor volume was >50 mm³ (i.p.) or 100 mm³ (i.t.). 100 μg of anti-PD-L1 (clone 10F.9G2) or isotype control antibody (clone LTF-2) were injected intraperitoneally (i.p.) one day before β-Lap treatment. Mice were then monitored for changes in tumor volumes (FIGS. 12A-B). Treatment with low dose of HPβCD-β-lap or anti-PD-L1 alone caused a slightly decreases tumor volume compared to vehicle, however, drug-combination therapies resulted in dramatic synergistic anti-tumor efficacy (FIGS. 12A-B).

Irradiation and NQO1 bioactivatable drug treatments synergistically promote antitumor immunity in mice. The inventors' previous results have shown that β-lap is a radiosensitizer in immunodeficient mice. Here the inventors examined the effects of β-lap on radiosensitizing NQO1+MC38 murine cancer cells in immunocompetent mice. MC38 NSCLC cancer cells that express high levels of NQO1 (130+15 Units) were very responsive as tested in subcutaneous tumors (100 mm³) in C57BL/6J WT mice with 10 Gy+0.1 mg β-lap for 3 intratumorally (i.t.) injections, with significant inhibition of tumor growth (p<0.01, FIG. 13A). Similarly, MC38 subcutaneous tumors (50 mm3) show significant synergistic responses to 10 Gy+β-lap (30 mg/kg, i.p.) given every other day for 6 injections (p<0.01, FIG. 13B). Mice have no significant methemoglobinemia or weight loss (data not shown).

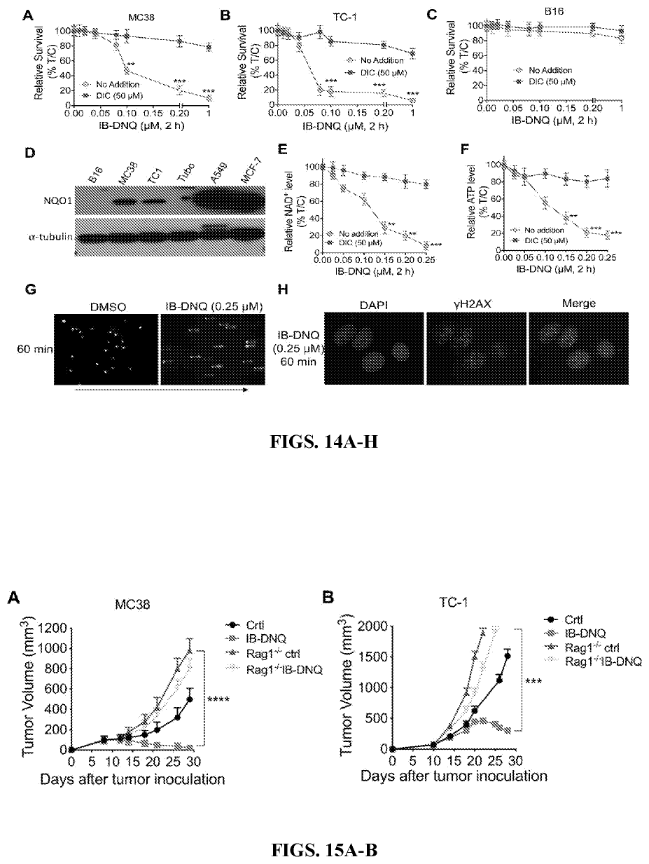

IB-DNQ kills murine cancer cells in an NQO1− dependent manner and induces NAD+/ATP depletion and DNA damage. IB-DNQ is a new more potent NQO1 bioactivatable drug. IB-DNQ was identical to β-lap, but 10- to 20-fold more potent, but was far less able to initiate methemoglobinemia (MH) compared to β-lap. Like β-lap, IB-DNQ also undergoes an NQO1-dependent futile redox cycle, which tries to detoxify the drug, forming its hydroquinone. It's unclear whether similar to β-lap, IB-DNQ-mediated tumor specific DNA damage and cell death have some interaction with the immune system. The inventors examined the antitumor activity of IB-DNQ in vitro. As shown, NQO1 was overexpressed in murine tumor cell lines (MC38 and TC-1) and deficient in B16 murine tumor cells lines (B16 and tubo) (FIG. 14A). IB-DNQ selectively killed NQO1⁺ murine tumor cell lines MC38 (FIG. 14B) and TC-1 (FIG. 14C), but not NQO1⁻ murine tumor cell B16 (FIG. 14D), and this effect can be suppressed by dicoumarol (DIC, a fairly specific NQO1 inhibitor) (FIGS. 14B-C) and do not occur in NQO1⁻ cells (B16) (FIG. 14D). The essential nucleotides NAD⁺ and ATP depletion were also noted after IB-DNQ treatment for 2 h in TC-1 cells, and dicoumarol can spare the effects (FIGS. 14E-F). After treatment with 0.25 μM IB-DNQ for 60 min, there was a dramatic increase in tail length indicating high DNA total damage (FIG. 14G), and DSBs marker, γ-H2AX foci was significantly increased (FIG. 14H) indicating double strand break.

IB-DNQ induces tumor regression dependent on the adaptive immune system. It is not known whether IB-DNQ stimulate antitumor immunity. Here the inventors separately established immunocompetent (C57BL/6J WT) and immunodeficient mice (rag^{-/-}) bearing NQO1-positive murine MC-38 colon cancer cells or TC-1 lung cancer cells. After 4 doses of IB-DNQ (0.15 mg) treatment, tumor regression occurred in MC-38 or TC-1 tumor-bearing WT mice, but not in adaptive immune-deficient rag^{-/-} mice (FIGS. 15A-B). This result suggested that adaptive immune system was also required for the profound antitumor efficacy of IB-DNQ in vivo.

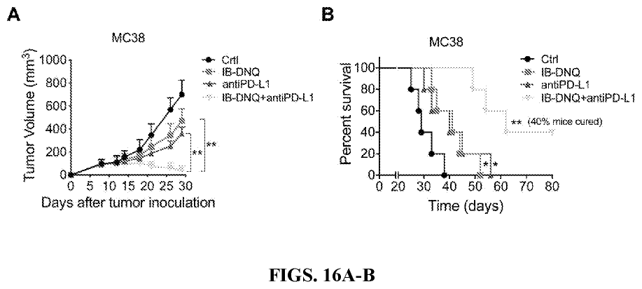

Synergy from combining IB-DNQ and immune checkpoint blockade (anti-PD-L1) therapies. The previous studies revealed β-lap can synergize with immune checkpoint blockade therapy. Here the inventors examine whether IB-DNQ can also synergize with immune checkpoint blockade therapy. MC38 tumor bearing mice (C57BL/6J WT) were treated intratumorally (i.t.) with HPβCD vehicle alone, anti-PD-L1 (Atezolizumab) or 0.05 mg (i.t.) of IB-DNQ with or without anti-PD-L1 every three days for 4 injections. Treatment was started when tumor volume was about 100 mm³. 100 μg of anti-PD-L1 (clone 10F.9G2) or isotype control antibody (clone LTF-2) were injected intraperitoneally (i.p.) one day before IB-DNQ treatment. Mice were then monitored for changes in tumor volumes and overall survival (FIGS. 16A-B). Similarly, the inventors found treatment with low dose of HPβCD-IB-DNQ or anti-PD-L1 alone also caused a significant decrease tumor growth and increase mice lifespan compared to vehicle, however, drug-combination therapies resulted in dramatic synergistic anti-tumor efficacy (FIGS. 16A-B).

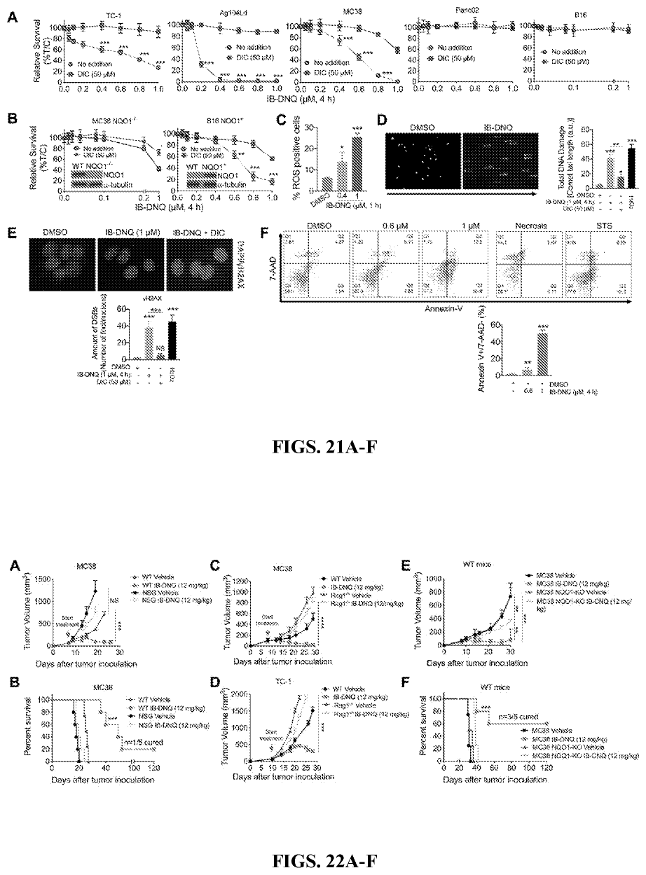

IB-DNQ induces tumor specific ROS formation and extensive DNA damage in an NQO1-dependent manner. NAD(P)H:quinone oxidoreductase 1 (NQO1) is a two-electron oxidoreductase elevated (>100-fold) in most solid cancers and has emerged as a promising target for direct tumor-killing. NQO1 can be exploited in a tumor-selective manner for therapy due to it specificity. The drug isobutyldeoxynyboquinone (IB-DNQ) has been shown significant antitumor effects on NQO1⁺ human solid cancers. However, it is still unclear whether IB-DNQ-mediated tumor specific DNA damage and cell death have some interaction with the immune system. To determine whether IB-DNQ selectively targets NQO1⁺ tumors and triggers immune responses, the inventors screened multiple murine cancer cell lines to investigate the antitumor effect of IB-DNQ in vitro. As shown in FIGS. 21A-B, IB-DNQ selectively killed NQO1⁺ murine tumor cell lines (TC-1, Ag104Ld, MC38 and B16 overexpressing NQO1), and this effect can be suppressed by dicoumarol (DIC, a fairly specific NQO1 inhibitor) or knocking out of NQO1 (FIG. 21B). Moreover, high ROS levels after a 1 h exposure to IB-DNQ (FIG. 21C) and DNA damage (total or double strand break) after a 4 h exposure to IB-DNQ (FIGS. 21D-E) were observed in NOQ1⁺ murine cancer cells. A further assay showed that, in the present of IB-DNQ, Annexin-V⁺-indicated apoptosis cell death was observed (FIG. 21F). These results indicated that IB-DNQ selectively induces NQO1⁺ tumor cell death through intensive tumor-specific ROS production and extensive DNA damage in vitro.

The antitumor efficacy of IB-DNQ in mice requires host immune system. To date, most studies focusing on the effect of IB-DNQ treatment on tumor cells death have targeted cancer cell autonomous mechanism. Here, the inventors hypothesized that IB-DNQ-mediated antitumor effects involves the activation of the immune response. To examine the role of immune responses in the antitumor effect of IB-DNQ, the inventors established MC38 subcutaneous syngeneic tumor models in immunocompetent and immunodeficient mice. On day 7 after tumor inoculation, IB-DNQ was intratumorally administered to tumor-bearing WT C57BL/6 or NOD.Cg-Prkdc^{scid} Il2rgt^{m1Wj1}/SzJ (NSG) mice (FIGS. 22A-B). Surprisingly, tumors were regressed after 4 does of IB-DNQ treatment in tumor-bearing WT group together with 20% cure fraction. By contrast, tumors in IB-DNQ-treated NSG mice showed only a minimal decrease in tumor size compared to vehicle. These results indicated that IB-DNQ-mediated antitumor effect involves the immune responses. To further study the effect of IB-DNQ on adaptive immunity, MC38 and TC-1 tumor cells were subcutaneously transplanted into WT and Rag1 KO (Rag1^{-/-}) C57BL/6 mice (FIGS. 22C-D). As shown and expectedly, Rag1 KO blocked the anti-tumor effects of IB-DNQ and tumors grew dramatically, suggesting that adaptive immune system was required for the profound antitumor efficacy of IB-DNQ in vivo. Moreover, consistent with the in vitro study, IB-DNQ therapeutic effect was abolished in NQO1 KO MC38 mouse models even though the tumor size was slightly decreased at the beginning of treatment (FIGS. 22E-F), indicating that NQO1 is essential and sufficient for IB-DNQ-mediated antitumor effect.

IB-DNQ treatment affect CD45⁺ immune cells population in the Tumor Microenvironment. To test which immune cell subset is essential for controlling the tumor burden and how tumor microenvironment is changed by IB-DNQ, the inventors investigated the immune microenvironment of MC38 tumors after IB-DNQ treatment. As shown, a significant increase in CD8⁺ and CD4⁺ T cells was observed in IB-DNQ-treated tumors compared to the control tumors (FIGS. 23A-B). Furthermore, IB-DNQ significantly increased MHC II⁺ DC proportion but had no effect on macrophages infiltration (FIGS. 23A-B). These results suggested that CD8⁺ and CD4⁺ T cells may play an essential role in the antitumor efficacy of IB-DNQ. To rule out direct effect of IB-DNQ on T cells, CD8⁺ T cells proliferation (FIG. 23C) and survival (FIG. 23D) after exposure to lethal dose of IB-DNQ were determined. Indeed, IB-DNQ had no effect on CD8⁺ T cell apoptosis and on anti-CD3/anti-CD28-stimulated cell proliferation.

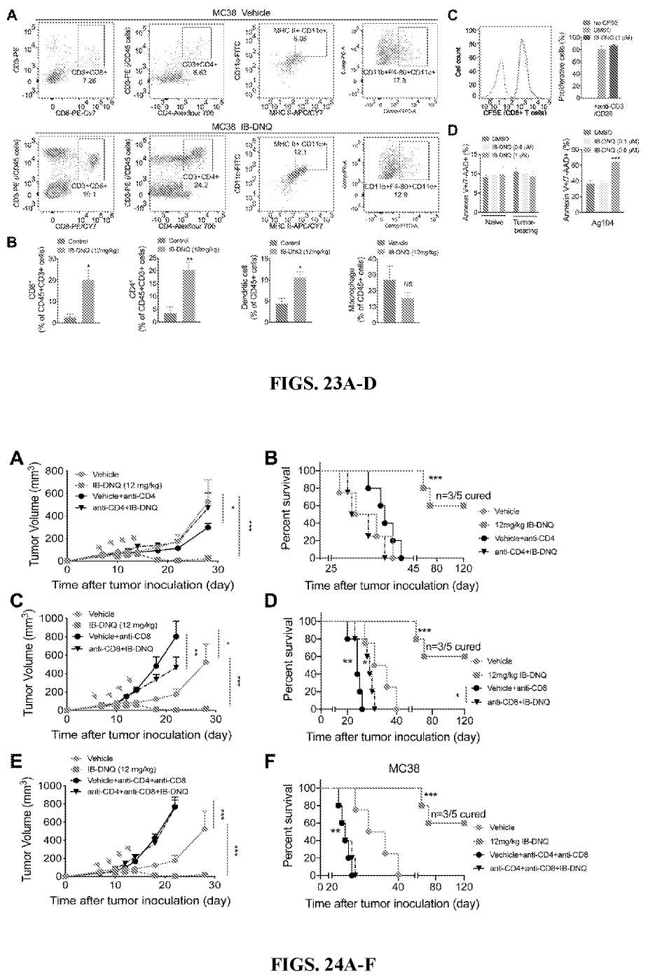

IB-DNQ-mediated-antitumor effect depends on CD8+ and CD4⁺ T cells. To investigate the roles of CD4⁺ and CD8⁺ T cells in IB-DNQ's effect, anti-tumor effect of IB-DNQ after CD4⁺ and/or CD8⁺ T cell depletion was examined. As expectedly, CD4⁺ and CD8⁺ T cell depletion totally abolished the anti-tumor effect of IB-DNQ (FIGS. 24E-F), but surprisingly, CD4⁺ or CD8⁺ T cell depletion alone partially blocked the IB-DNQ's effect (FIGS. 24A-D), indicating that both CD8⁺ and CD4⁺ T cells are required for IB-DNQ-mediated tumor regression.

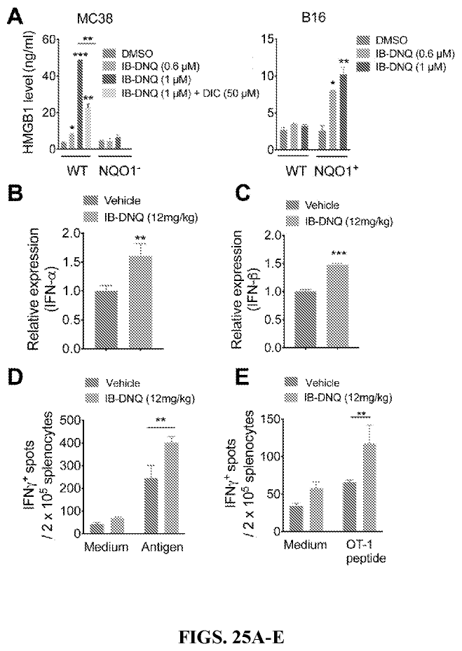

IB-DNQ induces tumor ICD and dendritic cell-mediated T cell cross-priming. The inventors' previous studies suggested that tumor treatment with some chemo-radiotherapies induced tumor cells immunogenic cell death (ICD) that promotes the antigenicity and immunogenicity of tumors. The immunogenicity of tumor cells dying via ICD is favored by cross-presentation of antigens by DCs to anti-tumor CD8 T-cells responsible for controlling the tumor. Given that CD8⁺ T cells play essential role in the IB-DNQ anti-tumor effect, thereby, IB-DNQ might potentially induce ICD in the tumor leading to antigen presentation to CD8⁺ T cells. To test this hypothesis, one of the ICD hallmarks, HMGB1, was checked after IB-DNQ treatment. Indeed, MC38 and B16 NQO1⁺ cells targeted by IB-DNQ secreted high levels of HMGB1, while MC38 NQO1^{-/-} and B16 (NQO1 deficient) cells that could not be induced cell death by IB-DNQ secreted less HMGB1 (FIG. 25A). As type 1 interferon (IFN) is essential for optimal cross-priming of T cells, type 1 IFNs were determined whether it was involved in T cell response mediated by IB-DNQ. As shown, expression of IFN-α and IFN-β was upregulated significantly in tumors with IB-DNQ treatment compared to the control (FIGS. 25B-C). Meanwhile, IFN-γ-indicated the antigen-specific T cell response was also observed (FIGS. 25D-E). In the presence of tumor antigen (irradiated tumor cells), IFN-γ-indicated T cells were dramatically increased in IB-DNQ treatment group (FIG. 25D). Similarly, in the IFN-γ ELISPOT assay with OT-1 peptide, the number of OT-1 specific T cells was significantly increased in IB-DNQ treatment group (FIG. 25E). Together, these results indicated that IB-DNQ induces ICD for cross-priming and activates T cells to suppress tumor growth.

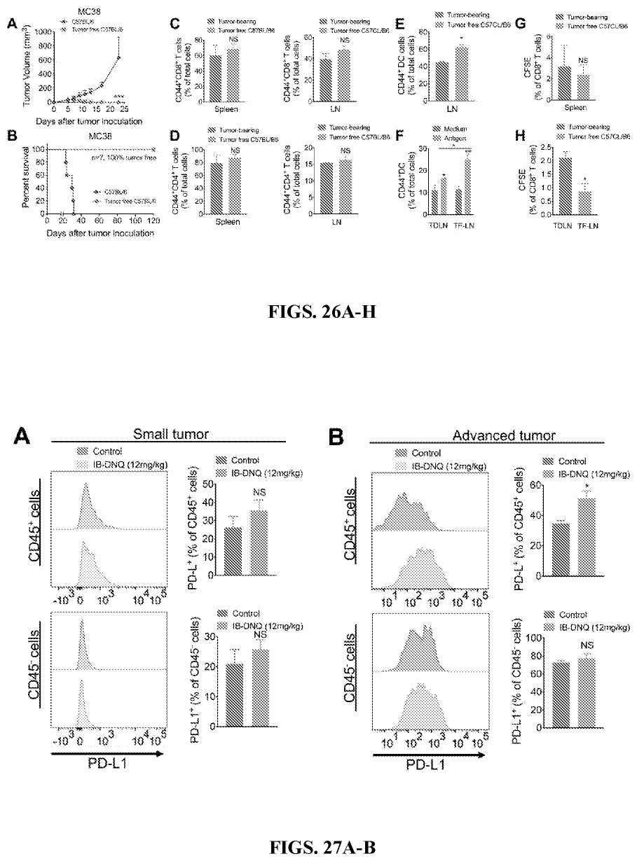

IB-DNQ induces innate immune memory instead of classical immunological memory. To investigate whether IB-DNQ-mediated anti-tumor responses result in prolonged protective T cell immunity, the mice that underwent complete MC38 tumor rejection after IB-DNQ treatment were rechallenged by MC38 cells (3×10⁶). Intriguing, all the IB-DNQ cured mice rejected the rechallenged tumors (FIGS. 26A-B), suggesting that memory T cells might be generated after IB-DNQ treatment. To further determine the generation of memory T cells, CD44⁺-indicated memory T cells were examined in the cured mice. 30 days later after rejection of the rechallenged tumors, different organs from the cured mice were isolated and single cells were analyzed by flow cytometry. Surprisingly, neither CD44⁺CD8⁺ nor CD44⁺CD4⁺ memory T cells were observed in spleen and LN (FIGS. 26C-D) and other organs (data not shown). Memory B cells were also investigated, but similar results as memory T cells were observed (data not shown). Interestingly, a significant increase of CD44⁺DCs was observed in cured mice (FIG. 26E). Several studies suggested that CD44 is important for the formation of DC-T cell tight conjugates and CD44 on DCs could affect T cell activation. Our results showed that, in the presence of antigen (irradiated or IB-DNQ-treated tumor cells), CD44⁺DCs either from tumor-bearing LN (TDLN) or tumor-free LN (TF-LN) were stimulated compared to the control, while TF-LN group showed more significant increase (FIG. 26F). Meanwhile, T cell proliferation was not significant increased when CD8⁺ T cells separated from spleen were co-cultured with irradiated tumor cells (FIG. 26G). However, when CD8⁺ T cells separated from naïve spleen were co-cultured with LN cells from tumor-bearing or tumor-free mice in the presence of antigen, T cell proliferation was significantly stimulated (FIG. 26H), indicating that CD44⁺DCs in TF-LN might play a critical role in T cells activation and proliferation. Together, all these results suggested that IB-DNQ anti-tumor effect might induce innate immune memory (memory-like dendritic cell responses, etc.) instead of classical immunological memory.

Programmed death-ligand 1 (PD-L1) expression is upregulated in mice with big tumor Burdens. Previous studies from other research groups demonstrate that Type I IFNs-induced upregulation of PD-L1 expression in the tumor microenvironment is one of the major reasons for acquired tumor resistance to multiple treatments. The inventors' previous studies indicate that IB-DNQ treatment results in upregulation of type I IFNs signaling in the tumor microenvironment (FIGS. 25B-C). To determine whether IB-DNQ treatment increases PD-L1 expression in TME, the inventors established two mouse models bearing small tumors (50 mm³) or advanced tumors (150 mm³). Tumors were collected 24 h later after the last IB-DNQ injection for flow cytometry analysis. Indeed, they found that PD-L1 expression was significantly upregulated by IB-DNQ treatment (12 mg/kg, i.v.) in CD45⁺ immune cells in mice with big tumor burdens (FIG. 27A), but not in mice with small tumor burdens (FIG. 27B). These findings suggest IB-DNQ treatment causes upregulation of PD-L1 levels within TME, which leads to the therapeutic window for combination therapy of IB-DNQ+PD-L1 inhibitors for advanced NQO1⁺ tumors.

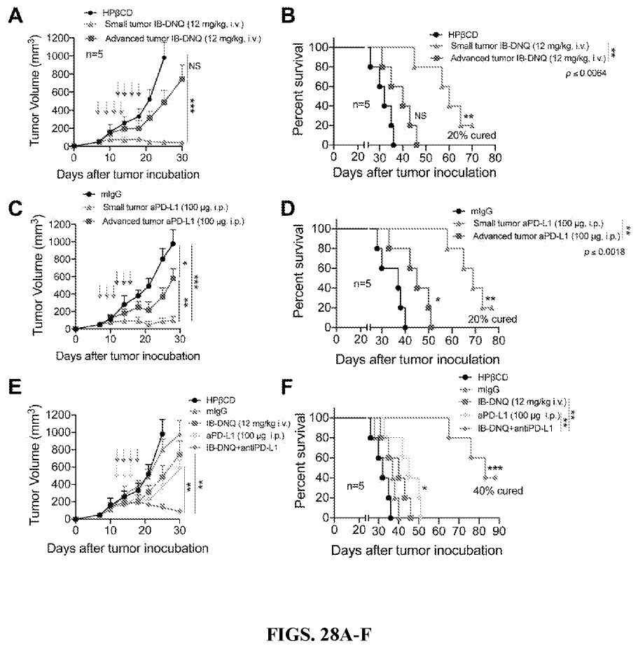

IB-DNQ overcomes checkpoint blockade resistance. Well-established tumors are subject to inherent resistance mechanisms and hard to achieve complete tumor rejection. To investigate if large established tumors are sensitive to IB-DNQ or PD-L1 blockade treatment alone, the inventors generated small (about 50 mm³) and large (about 150 mm³) tumor burdens in C57BL/6 WT mice (12 mg/kg of IB-DNQ, i.v. or 100 μg of anti-PD-L1, i.p.). Tumor volumes and long-term survival were monitored. The found that complete tumor rejection was only achieved in mice bearing small but not large established tumors after the treatment of IB-DNQ or anti-PD-1 alone (FIGS. 28A-D). Based on previous studies (FIG. 27), the inventors propose that IB-DNQ synergizes with immune checkpoint blockade therapy to efficaciously kill well-established NQO1⁺ tumors. To test this hypothesis, they combined IB-DNQ (12 mg/kg, i.v.) treatment with anti-PD-L1 treatment (100 μg, i.p.) in mice bearing large established MC38 tumors. They found that the advanced MC38 tumors only showed a moderate response to IB-DNQ or anti-PD-L1 alone (FIGS. 28E-F). In stark contrast, mice in the combination groups showed robust tumor control and regression (FIGS. 28E-F). Notably, 40% of tumor-bearing mice completely rejected their tumors on combination treatment by systemic treatment. These findings suggested that increased PD-L1 within TME contributes to tumor relapse of large tumors after initial responses to IB-DNQ, and combination therapy of IB-DNQ with immune checkpoint blockade eradicates the advanced and checkpoint refractory tumors.

### Example 3—Discussion

Lack of proper innate sensing may limit T-cell-targeted immunotherapy (Patel et al., 2018; Qiao et al., 2017; Gajewski et al., 2013). The inventors hypothesized that induction of immunogenic innate sensing via some tumor-targeting genotoxic agents might induce antitumor immunity and overcome immune checkpoint blockade resistance. Here, they used several syngeneic immunocompetent mouse models and immune reconstituted human xenograft models that closely recapitulate human disease to evaluate the antitumor efficacy of NQO1 bioactivatable β-lapachone (β-lap). They demonstrated that β-lap had an impressive antitumor effect in vivo, largely depending on innate and adaptive immunity. They discovered that after activation by NQO1, β-lap caused tumor-selective cell death and induced innate sensing for adaptive antitumor immunity. Mechanistically, tumor β-lap triggered immunogenic cell death and increased tumor immunogenicity by the release of HMGB1. This activated the innate immune response and induced a type I IFN signature in TLR4/MyD88 dependent manner, which stimulated antitumor T cell adaptive immunity and restrained tumor growth. Importantly, β-lap overcame immunotherapy resistance. When combined with anti-PDL1 mAbs, β-lap further enhanced CD8 T cell infiltration and antigen specific T cell response and eradicated large established and checkpoint blockade refractory tumors.

In preclinical and clinical studies, checkpoint blockade essentially takes the “brakes” off the immune system and has proven insufficient to break tolerance, unless co-administered with certain “fuels” to activate local immune activities and induce desired T cell responses (Salmon et al., 2016; Kleponis et al., 2015; Kamphorst et al., 2017). Stimulating the innate immune sensing in combination with T cell checkpoint immunotherapy might be one answer. Natural innate immune sensing of tumors appears to occur via antigen uptake and presentation, host PRR pathway, type I IFN production, and cross-priming of T cells (Woo et al., 2015). Normally, innate immune cells can contribute to tumor control either directly or indirectly, through DCs activation or production of cytokines that support effector T cell differentiation. However, tumors also avoid immune clearance by silencing PRR signaling or subverting PRR signals and accumulating dysfunctional innate cells to promote cancer suppressive inflammation rather than priming adaptive immune response (Lee et al., 2009; Hernandez et al., 2016; Givennikov et al., 2010). Thus, the presence or absence of appropriate innate sensing in the tumor microenvironment may in fact be a critical determinant of checkpoint blockade therapeutic success.

One approach to induce immunogenic innate sensing and reshape the tumor microenvironment is to use genotoxic agents such as chemotherapies that are already widely employed in cancer treatment (Patel et al., 2018; Emens et al., 2015; Pfirschke et al., 2016). Dying tumor cells due to cancer therapeutics can express or release DAMPs for activation of immune cells via specific innate sensing pathways and elicit antitumor immune responses against tumor-associated antigens. Indeed, combining immune checkpoint blockade with chemotherapy is being extensively studied in clinical trials (Garg et al., 2017; Langer et al., 2016; Gandhi et al., 2018; Weiss et al., 2017). However, one of the major limitations in the use of traditional chemotherapy drugs arises from their lack of selectivity and related adverse toxicity to nontargeted tissues, especially the adaptive immune system. NQO1 is a two-electron oxidoreductase expressed in multiple tumor types at levels 5- to 200-fold above normal tissue, and is a potential therapeutic target (Huang et al., 2016; Li et al., 2016). β-Lap is a new class of NQO1-targeted drug which can be catalyzed by NQO1 to generate reactive oxygen species (ROS) (Huang et al., 2016; Doskey et al., 2016). Indeed, the inventors found that β-lap selectively killed tumors highly expressing NQO1 both in vitro and in vivo, and this killing effect was abolished when the inventors knocked out NQO1, indicating the ideal selectivity of this drug. Although for a long time genotoxic agents were assumed to exert their effects mostly via cancer cell-autonomous mechanisms, i.e., by directly inhibiting the proliferation or triggering the demise of malignant cells, accumulating evidence indicates that multiple chemotherapeutics that have been successfully employed in the clinic for decades also trigger immunogenic cell death (ICD) and elicit novel anticancer immune responses (Sistigu et al., 2014). Unfortunately, most ICD inducing agents have severe side effects at therapeutic doses due to lack of tumor-selectivity and severe immunosuppression (depleting rapidly dividing immune-cell population). Importantly, β-lap distinguished itself from other chemotherapeutic agents out of its ideal target effect on NQO1 overexpressing tumors and no immunosuppression on cytotoxic immunity. The inventors proved that tumor infiltrated CD8 β-lap has no cytotoxic effect on native and activated CD8 T cell even at the tumor-lethal dose. A novel question is whether NQO1 bioactivatable β-lap-mediated tumor-specific cell death has some interaction with the immune system; whether this “targeted” chemotherapeutic drug evokes an immunogenic cell death and triggers innate sensing. Currently, the inventors found that β-lap-induced NQO1+ tumor regression largely depends on host CD8⁺ T cells: the drug failed to control tumor progression in mice lacking these cells (Rag1−/− mice as well as wild type mice depleted with anti-CD8 antibodies). The inventors further proved that β-lap induced immunogenic cell death and activated innate sensing via an HMGB1/TLR4 pathway and unregulated the type I IFNs signaling in the tumor microenvironment, resulting in the promotion of Batf3 DCs to cross-prime T cells and activate of antitumoral adaptive immune response.

Reducing tumor burden and increasing tumor immunogenicity are believed to be two key

factors to improve immunotherapy (Zappasodi et al., 2018). Notably, β-lap promoted HMGB1-dependent immunogenicity and activated innate sensing to bridge innate and adaptive immune response, and markedly shrunk the tumor mass, and is thus a promising partner for combination with immunotherapy. Indeed, the inventors demonstrated that β-lap managed to eradicate large established and checkpoint blockade refractory MC38 and B16 tumors by combination with anti-PD-L1 immunotherapy and dramatically increased the survival rate. They further proved that combination therapy dramatically increased the tumor-infiltrating lymphocytes and tumor-antigen specific T cells as well as CD8/Treg ratio in the tumor microenvironment, as compared to β-lap or anti-PDL1 monotherapy. Future work is needed to explore the optimal dose of compound and scheduling and sequencing of a combination of these combinations.

Overall, this study has provided a novel insight into how β-lap, a unique targeted chemotherapeutic drug, induces antitumor effect through coordinated innate and adaptive immunity and how β-lap treatment sets up an ideal microenvironment for immunotherapy. β-lap is currently being tested in monotherapy or in combination with the other chemodrugs in patients with NQO1+ solid tumors. This study points out that β-lap's innate sensing capability can prepare NQO1+ patients for a successful response to immunotherapy.

### ************

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments, exemplary embodiments and optional features, modification and variation of the concepts herein disclosed may be resorted to by those skilled in the art, and that such modifications and variations are considered to be within the scope of this disclosure as defined by the appended claims. The specific embodiments provided herein are examples of useful embodiments of the present disclosure and it will be apparent to one skilled in the art that the present disclosure may be carried out using a large number of variations of the devices, device components, methods steps set forth in the present description. As will be recognized by one of skill in the art, methods and devices useful for the present methods can include a large number of optional composition and processing elements and steps.

### V. REFERENCES

The following references, to the extent that they provide exemplary procedural or other details supplementary to those set forth herein, are specifically incorporated herein by reference:
1. Topalian S L, Drake C G, Pardoll D M. Immune checkpoint blockade: a common denominator approach to cancer therapy. Cancer cell. 2015; 27(4):450-61.
2. Pauken K E, Wherry E J. Overcoming T cell exhaustion in infection and cancer. Trends in immunology. 2015; 36(4):265-76.
3. Sharma P, Hu-Lieskovan S, Wargo J A, Ribas A. Primary, Adaptive, and Acquired Resistance to Cancer Immunotherapy. Cell. 2017; 168(4):707-23.
4. Jenkins R W, Barbie D A, Flaherty K T. Mechanisms of resistance to immune checkpoint inhibitors. British journal of cancer. 2018; 118(1):9-16.
5. Garber K. A new cancer immunotherapy suffers a setback. Science. 2018; 360(6389):588.
6. Lee Y, Auh S L, Wang Y, Burnette B, Wang Y, Meng Y, et al. Therapeutic effects of ablative radiation on local tumor require CD8⁺ T cells: changing strategies for cancer treatment. Blood. 2009; 114(3):589-95.
7. Woo S R, Corrales L, Gajewski T F. Innate immune recognition of cancer. Annual review of immunology. 2015; 33:445-74.
8. Takeuchi O, Akira S. Pattern recognition receptors and inflammation. Cell. 2010; 140(6): 805-20.
9. Patel S A, Minn A J. Combination Cancer Therapy with Immune Checkpoint Blockade: Mechanisms and Strategies. Immunity. 2018; 48(3):417-33.
10. Hernandez C, Huebener P, Schwabe R F. Damage-associated molecular patterns in cancer: a double-edged sword. Oncogene. 2016; 35(46):5931-41.
11. Grivennikov S I, Greten F R, Karin M. Immunity, inflammation, and cancer. Cell. 2010; 140(6): 883-99.
12. Garg A D, More S, Rufo N, Mece O, Sassano M L, Agostinis P, et al. Trial watch: Immunogenic cell death induction by anticancer chemotherapeutics. Oncoimmunology. 2017; 6(12):e1386829.
13. Sistigu A, Yamazaki T, Vacchelli E, Chaba K, Enot D P, Adam J, et al. Cancer cell-autonomous contribution of type I interferon signaling to the efficacy of chemotherapy. Nature medicine. 2014; 20(11):1301-9.
14. Galluzzi L, Buque A, Kepp O, Zitvogel L, Kroemer G. Immunological Effects of Conventional Chemotherapy and Targeted Anticancer Agents. Cancer cell. 2015; 28(6):690-714.
15. Li R, Bianchet M A, Talalay P, Amzel L M. The three-dimensional structure of NAD(P)H:quinone reductase, a flavoprotein involved in cancer chemoprotection and chemotherapy: mechanism of the two-electron reduction. Proceedings of the National Academy of Sciences of the United States of America. 1995; 92(19):8846-50.
16. Oh E T, Kim J W, Kim J M, Kim S J, Lee J S, Hong S S, et al. NQO1 inhibits proteasome-mediated degradation of HIF-1alpha. Nature communications. 2016; 7:13593.
17. Li Z, Zhang Y, Jin T, Men J, Lin Z, Qi P, et al. NQO1 protein expression predicts poor prognosis of non-small cell lung cancers. BMC cancer. 2015; 15:207.
18. Ma Y, Kong J, Yan G, Ren X, Jin D, Jin T, et al. NQO1 overexpression is associated with poor prognosis in squamous cell carcinoma of the uterine cervix. BMC cancer. 2014; 14:414.
19. Huang X, Motea E A, Moore Z R, Yao J, Dong Y, Chakrabarti G, et al. Leveraging an NQO1 Bioactivatable Drug for Tumor-Selective Use of Poly(ADP-ribose) Polymerase Inhibitors. Cancer cell. 2016; 30(6):940-52.
20. Pink J J, Planchon S M, Tagliarino C, Varnes M E, Siegel D, Boothman D A. NAD(P)H:Quinone oxidoreductase activity is the principal determinant of beta-lapachone cytotoxicity. The Journal of biological chemistry. 2000; 275(8):5416-24.
21. Doskey C M, Buranasudja V, Wagner B A, Wilkes J G, Du J, Cullen J J, et al. Tumor cells have decreased ability to metabolize H₂O₂: Implications for pharmacological ascorbate in cancer therapy. Redox biology. 2016; 10:274-84.
22. Huang X, Dong Y, Bey E A, Kilgore J A, Bair J S, Li L S, et al. An NQO1 substrate with potent antitumor activity that selectively kills by PARP1-induced programmed necrosis. Cancer research. 2012; 72(12):3038-47.
23. Li L S, Reddy S, Lin Z H, Liu S, Park H, Chun S G, et al. NQO1-Mediated Tumor-Selective Lethality and Radiosensitization for Head and Neck Cancer. Molecular cancer therapeutics. 2016; 15 (7): 1757-67.
24. Tang H, Wang Y, Chlewicki L K, Zhang Y, Guo J, Liang W, et al. Facilitating T Cell Infiltration in Tumor Microenvironment Overcomes Resistance to PD-L1 Blockade. Cancer cell. 2016; 29(3):285-96.
25. Morton J J, Bird G, Refaeli Y, Jimeno A. Humanized Mouse Xenograft Models: Narrowing the Tumor-Microenvironment Gap. Cancer research. 2016; 76(21):6153-8.
26. Tang H, Liang Y, Anders R A, Taube J M, Qiu X, Mulgaonkar A, et al. PD-L1 on host cells is essential for PD-L1 blockade-mediated tumor regression. The Journal of clinical investigation. 2018; 128(2):580-8.
27. Sanchez-Paulete A R, Cueto F J, Martinez-Lopez M, Labiano S, Morales-Kastresana A, Rodriguez-Ruiz M E, et al. Cancer Immunotherapy with Immunomodulatory Anti-CD137 and Anti-PD-1 Monoclonal Antibodies Requires BATF3-Dependent Dendritic Cells. Cancer discovery. 2016; 6(1):71-9.
28. Broz M L, Binnewies M, Boldajipour B, Nelson A E, Pollack J L, Erle D J, et al. Dissecting the Tumor Myeloid Compartment Reveals Rare Activating Antigen-Presenting Cells Critical for T Cell Immunity. Cancer cell. 2014; 26(6):938.
29. Salmon H, Idoyaga J, Rahman A, Leboeuf M, Remark R, Jordan S, et al. Expansion and Activation of CD103(+) Dendritic Cell Progenitors at the Tumor Site Enhances Tumor Responses to Therapeutic PDL1 and BRAF Inhibition. Immunity. 2016; 44(4):924-38.
30. Corrales L, Matson V, Flood B, Spranger S, Gajewski T F. Innate immune signaling and regulation in cancer immunotherapy. Cell research. 2017; 27(1):96-108.
31. Deng L, Liang H, Xu M, Yang X, Burnette B, Arina A, et al. STING-Dependent Cytosolic DNA Sensing Promotes Radiation-Induced Type I Interferon-Dependent Antitumor Immunity in Immunogenic Tumors. Immunity. 2014; 41(5):843-52.
32. Zitvogel L, Galluzzi L, Kepp O, Smyth M J, Kroemer G. Type I interferons in anticancer immunity. Nature reviews Immunology. 2015; 15(7):405-14.
33. Apetoh L, Ghiringhelli F, Tesniere A, Obeid M, Ortiz C, Criollo A, et al. Toll-like receptor 4-dependent contribution of the immune system to anticancer chemotherapy and radiotherapy. Nature medicine. 2007; 13(9):1050-9.
34. Smyth M J, Ngiow S F, Ribas A, Teng M W. Combination cancer immunotherapies tailored to the tumour microenvironment. Nature reviews Clinical oncology. 2016; 13(3):143-58.
35. Mathios D, Kim J E, Mangraviti A, Phallen J, Park C K, Jackson C M, et al. Anti-PD-1 antitumor immunity is enhanced by local and abrogated by systemic chemotherapy in GBM. Science translational medicine. 2016; 8(370):370ra180.
36. Ariyan C E, Brady M S, Siegelbaum R H, Hu J, Bello D M, Rand J, et al. Robust Antitumor Responses Result from Local Chemotherapy and CTLA-4 Blockade. Cancer immunology research. 2018; 6(2):189-200.
37. Chen S, Lee L F, Fisher T S, Jessen B, Elliott M, Evering W, et al. Combination of 4-1BB agonist and PD-1 antagonist promotes antitumor effector/memory CD8 T cells in a poorly immunogenic tumor model. Cancer immunology research. 2015; 3(2):149-60.
38. Curran M A, Montalvo W, Yagita H, Allison J P. PD-1 and CTLA-4 combination blockade expands infiltrating T cells and reduces regulatory T and myeloid cells within B16 melanoma tumors. Proceedings of the National Academy of Sciences of the United States of America. 2010; 107(9):4275-80.
39. Qiao J, Tang H, Fu Y X. DNA sensing and immune responses in cancer therapy. Current opinion in immunology. 2017; 45:16-20.
40. Gajewski T F, Schreiber H, Fu Y X. Innate and adaptive immune cells in the tumor microenvironment. Nature immunology. 2013; 14(10):1014-22.
41. Kleponis J, Skelton R, Zheng L. Fueling the engine and releasing the break: combinational therapy of cancer vaccines and immune checkpoint inhibitors. Cancer biology & medicine. 2015; 12(3):201-8.
42. Kamphorst A O, Wieland A, Nasti T, Yang S, Zhang R, Barber D L, et al. Rescue of exhausted CD8 T cells by PD-1-targeted therapies is CD28-dependent. Science. 2017; 355(6332):1423-7.
43. Emens L A, Middleton G. The interplay of immunotherapy and chemotherapy: harnessing potential synergies. Cancer immunology research. 2015; 3(5):436-43.
44. Pfirschke C, Engblom C, Rickelt S, Cortez-Retamozo V, Garris C, Pucci F, et al. Immunogenic Chemotherapy Sensitizes Tumors to Checkpoint Blockade Therapy. Immunity. 2016; 44(2):343-54.
45. Langer C J, Gadgeel S M, Borghaei H, Papadimitrakopoulou V A, Patnaik A, Powell S F, et al. Carboplatin and pemetrexed with or without pembrolizumab for advanced, non-squamous non-small-cell lung cancer: a randomised, phase 2 cohort of the open-label KEYNOTE-021 study. The Lancet Oncology. 2016; 17(11):1497-508.
46. Gandhi L, Rodriguez-Abreu D, Gadgeel S, Esteban E, Felip E, De Angelis F, et al. Pembrolizumab plus Chemotherapy in Metastatic Non-Small-Cell Lung Cancer. The New England journal of medicine. 2018; 378(22):2078-92.
47. Weiss G J, Waypa J, Blaydorn L, Coats J, McGahey K, Sangal A, et al. A phase Ib study of pembrolizumab plus chemotherapy in patients with advanced cancer (PembroPlus). British journal of cancer. 2017; 117(1):33-40.
48. Zappasodi R, Merghoub T, Wolchok J D. Emerging Concepts for Immune Checkpoint Blockade-Based Combination Therapies. Cancer cell. 2018; 33(4):581-98.
49. Lundberg A P, Francis J M, Pajak M, Parkinson E I, Wycislo K L, Rosol T J et al. Pharmacokinetics and derivation of an anticancer dosing regimen for the novel anti-cancer agent isobutyl-deoxynyboquinone (IB-DNQ), a NQO1 bioactivatable molecule, in the domestic felid species. Invest New Drugs. 2017; 35(2):134-44.
50. Li X, Liu Z, Zhang A, Han C, Shen A, Jiang L, et al. NQO1 targeting prodrug triggers innate sensing to overcome checkpoint blockade resistance. Nat Communications. 2019; 10(1):3251.

1. A method of killing or inhibiting the growth of cancer cells in a patient having cancer comprising administering a NQO1 bioactivatable drug in combination with a checkpoint inhibitor.

2-3. (canceled)

4. The method of claim 1, wherein the cancer cells have base excision repair (BER) defects or vulnerabilities due to faulty DNA repair processes.

5. The method of claim 4, wherein the BER defect or vulnerability comprises defective levels of X-ray cross complementing 1 or XRCC1 gene/protein/enzyme.

6. The method of claim 1, wherein the NQO1 bioactivatable drug is used in combination with a small molecule checkpoint inhibitor or an antibody checkpoint inhibitor.

7. (canceled)

8. The method of claim 1, wherein use of the NQO1 bioactivatable drug is in combination with an inhibitor of PD-1 or CTLA-4.

9. The method of claim 6, wherein the NQO1 bioactivatable drug is β-lapachone or a DNQ compound.

10. (canceled)

11. The method of claim 8, wherein the NQO1 bioactivatable drug is β-lapachone or a DNQ compound.

12. The method of claim 1, further comprising an additional chemotherapeutic agent or radiotherapy.

13. The method of claim 1, wherein the cancer cells have elevated levels of NQO1.

14. The method of claim 1, wherein the cancer cells are in the form of a solid tumor.

15. The method of claim 14, wherein the cancer cells are non-small cell lung cancer cells, prostate cancer cells, pancreatic cancer cells, breast cancer cells, head and neck cancer cells, or colon cancer cells.

16. The method of claim 1, wherein the NQO1 bioactivatable drug is: 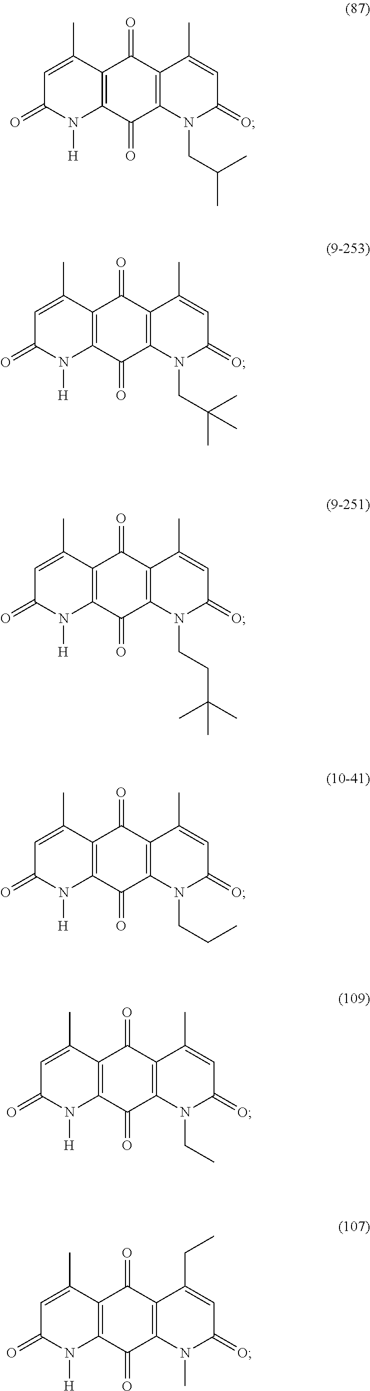 [SMILES: Cc1cc(=O)[nH]c2c1C(=O)c1c(C)cc(=O)n(CC(C)C)c1C2=O ; Cc1cc(=O)[nH]c2c1C(=O)c1c(C)cc(=O)n(CC(C)(C)C)c1C2=O ; Cc1cc(=O)[nH]c2c1C(=O)c1c(C)cc(=O)n(CCC(C)(C)C)c1C2=O ; CCCn1c2c(c(C)cc1=O)C(=O)c1c(C)cc(=O)[nH]c1C2=O ; CCn1c2c(c(C)cc1=O)C(=O)c1c(C)cc(=O)[nH]c1C2=O ; CCc1cc(=O)n(C)c2c1C(=O)c1c(C)cc(=O)[nH]c1C2=O] 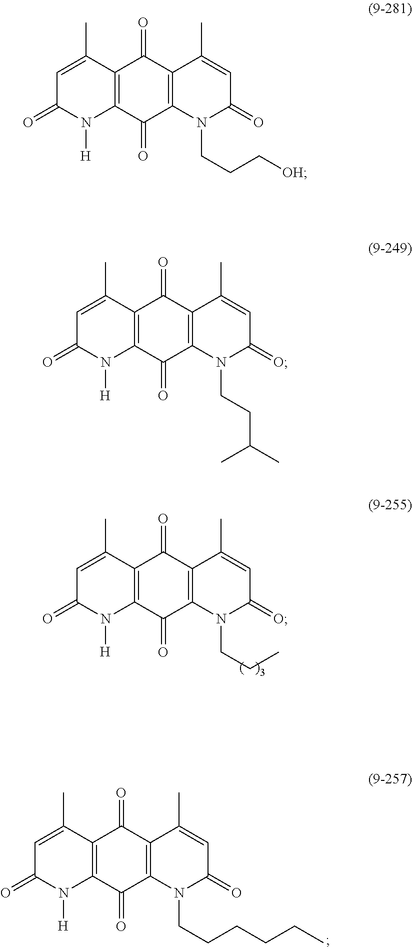
or a salt or solvate thereof.

17. (canceled)

18. The method of claim 1, wherein the NQO1 bioactivatable drug is DNQ or DNQ-87.

19. The method of claim 1, wherein the NQO1 bioactivatable drug is administered before the checkpoint inhibitor.

20. The method of claim 1, wherein the NQO1 bioactivatable drug is administered after the checkpoint inhibitor.

21. The method of claim 1, wherein the NQO1 bioactivatable drug is administered concurrent with the checkpoint inhibitor.

22. The method of claim 1, wherein the NQO1 bioactivatable drug is administered more than once.

23. The method of claim 1, wherein the checkpoint inhibitor is administered more than once.

24. The method of claim 1, wherein the NQO1 bioactivatable drug and the checkpoint inhibitor are administered more than once.

25. The method of claim 1, further comprising an additional anti-cancer therapy.

26. The method of claim 25, wherein the additional anti-cancer therapy may be a chemotherapy, a radiotherapy, an immunotherapy, a toxin therapy or surgery.
